(19) United States
(12) Patent Application Publication
(10) Pub. No.: US 2010/0144688 A1
(43) Pub. Date: Jun. 10, 2010

(54) 23-Substituted Derivatives of Lupane-type Pentacyclic Triterpenoids

(75) Inventors: Ali Koohang, Plainfield, IL (US); Aye Aye Mar, Tinley Park, IL (US); Erika Szotek, Hanover Park, IL (US); Ze-Qi Xu, Woodridge, IL (US); David A. Eiznhamer, Bloomingdale, IL (US); Michael T. Flavin, Darien, IL (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: ADVANCED LIFE SCIENCES, INC., Woodridge, IL (US)

(21) Appl. No.: 12/634,305

(22) Filed: Dec. 9, 2009

Related U.S. Application Data
(60) Provisional application No. 61/193,615, filed on Dec. 10, 2008.

(51) Int. Cl. A61K 31/568; C07J 53/00; C07J 17/00; C07J 1/00; A61K 31/58; A61P 31/12; A61P 35/00

(52) U.S. Cl. 514/172; 552/510; 549/416; 514/169

(57) ABSTRACT

The present invention comprises small molecule inhibitors of cell proliferative conditions, in particular cancer and conditions associated with cancer. For example, associated malignancies include ovarian cancer, cervical cancer, breast cancer, colorectal cancer, and glioblastomas, among others. Accordingly, the compounds of the present invention are useful for treating, preventing, and/or inhibiting these diseases. Thus, the present invention also comprises pharmaceutical formulations comprising the compounds and methods of using the compounds and formulations to inhibit cancer and treat, prevent, or inhibit the foregoing diseases.

### CROSS-REFERENCE TO OTHER APPLICATIONS

This application claims the benefit of U.S. Provisional Application No. 61/193,615 filed Dec. 10, 2008, which is incorporated by reference in its entirety.

### FIELD OF THE INVENTION

This invention relates to the field of inhibitors of cell proliferative conditions. In particular, the invention relates to inhibitors of cancer and conditions associated with cancer.

### BACKGROUND OF THE INVENTION

23-Hydroxybetulinic acid (1) is a natural product derived from the tissue culture of Paeonia japonica¹ and the roots of Pulsatilla chinensis (Bunge) Regel,² the latter of which is one of the most widely used Chinese traditional medicines. 23-Hydroxybetulinic acid is structurally related to another antitumor natural product betulinic acid (2)³ and can be considered a naturally occurring derivative of the latter compound.

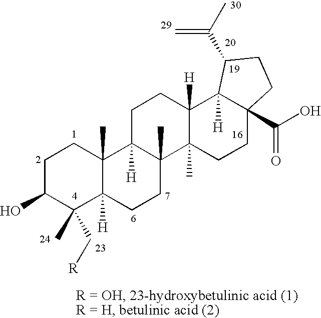

23-Hydroxybetulinic acid (1) has been reported to have similar cytotoxic activity to that of betulinic acid (2) against K-562 human leukemia and human HeLa cells,⁴ but with increased potency against murine melanoma B16 cells.⁵ 23-Hydroxybetulinic acid (1) induces growth arrest and apoptotic cell death by a decrease in telomerase activity and down-regulation of the anti-apoptotic gene bcl-2,⁶ as shown in human leukemia HL-60 cells, or by a rapid increase in reactive oxidative species production and a concomitant dissipation of mitochondrial membrane potential, as reported in murine melanoma B16 cells.⁵ Cellular uptake of 23-hydroxybetulinic acid (1) by human colon carcinoma cell lines (Caco-2) reached intracellular concentration of 906.7 μM/mg protein at approximately 60 minutes.⁷

Other naturally occurring lupane-type pentacyclic triterpenoids reported with substituent at 23-pisition includes 3-epi-23-hydroxybetulinic acid (3) isolated from the leaves of Acanthopanax gracilistylus,⁸ 23-hydroxybetulonic acid (4) isolated from the roots of Pulsatilla chinensis (Bunge) Regel,² Ilekudinol C (5) isolated from both the leaves of Ilex kudincha⁹ and the woody parts of Chaenomeles sinensis KOEHNE,¹⁰ Swinniol (6) isolated from the bark of Bersama swinnyi,¹¹ and 3α-hydroxy-23-carboxylic acid derivative (7) isolated from the leaves of Schefflera octophylla.¹² Several semi-synthetic derivatives of 23-hydroxybetulinic acid have also been reported such as 8 and 9.^{13,14}

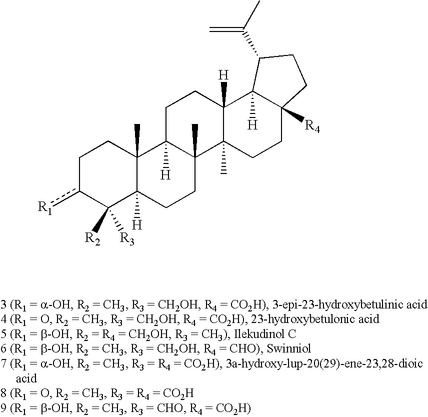

Supplies of 23-hydroxybetulinic acid (1) as well as other natural products from plant materials, however, are extremely limited and difficult to obtain. There has been no report of the synthesis of 23-hydroxybetulinic acid (1) in the literature.

In our continuing efforts to search for novel anticancer agents, we have embarked upon the design, synthesis and evaluation of 23-hydroxybetulinic acid (1), other naturally occurring 23-substituted lupane-type pentacyclic triterpenoids, and novel synthetic derivatives thereof.

### SUMMARY OF THE INVENTION

The invention provides compounds, methods and pharmaceutical compositions comprising the compounds useful for preventing and treating diseases such as cancer. The lupane-type triterpenoids of the invention inhibit cell proliferations, in particular cancer and conditions associated with cancer. For example, associated malignancies include ovarian cancer, cervical cancer, breast cancer, colorectal cancer, and glioblastomas, among others. Accordingly, the compounds of the invention are useful for treating, preventing, and/or inhibiting these diseases. Thus, the invention also comprising pharmaceutical formulations comprising the compounds and methods of using the compounds and formulations to inhibit cancer and treat, prevent, or inhibit the foregoing diseases.

In one aspect, the invention provides compound of the formula

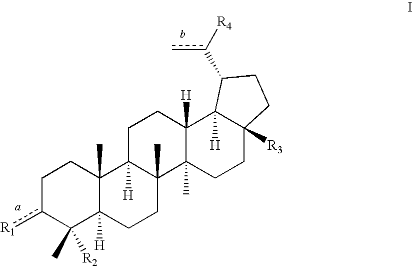

or a pharmaceutically acceptable salt thereof, wherein

each [symbol] is independently a single or double bond;

when bond a is a double bond, R₁ is ═O, ═S, ═N—OH, ═N—OR₅, or ═N—OCOR₅; or when bond a is a single bond, R₁ is H, halogen, NH₂, OH, SH, NHOH, NHR₅, NH(CH₂)ₙX, NR₅R₆, OR₅, OCOR₅, OCO(CH₂)ₙX, OC(O)OR₅, OCO(HC═CH)ₙR₅, OC(O)NHR₅, OC(O)NR₅R₆, OSO₂H, OSO₃H, OSO₂(R₅), OSO₂(CH₂)ₙX, OSi(R₅)ₘ(R₆)₃₋ₘ, SR₅, SCOR₅, SCO(CH₂)ₙX, SC(O)NH₂, SC(O)NHR₅, SC(O)NR₅R₆, NHCOR₅, NHC(O)OR₅, N(R₆)C(O)OR₅, NHC(O)NH₂, NHC(O)NHR₅, NHC(O)NR₅R₆, N(R₆)C(O)NH₂, N(R₆)C(O)NHR₅, N(R₆)C(O)NR₅R₆;

R₂ and R₃ are independently selected from CH₂NH₂, CH₂OH, CH₂SH, CH₂SePh, CH₂X, CH₂NHOH, CN, CHO, CO₂H, CH₂NHOR₅, CH═N—OH, CH═N—OR₅, CH═N—OCOR₅, CH₂NHR₅, CH₂NH(CH₂)ₙX, CH₂NR₅R₆, CH₂OR₅, CH₂OCOR₅, CH₂OC(O)OR₅, CH₂OCO(CH₂)ₙX, CH₂OCO(HC═CH)ₙR₅, CH₂OC(O)NH₂, CH₂OC(O)NHR₅, CH₂OC(O)NR₅R₆, CH₂OSO₂(R₅), CH₂OSO₂(CH₂)ₙX, CH₂OSi(R₅)ₘ(R₆)₃₋ₘ, CH₂SR₅, CH₂SCOR₅, CH₂SCO(CH₂)ₙX, CH₂SC(O)NH₂, CH₂SC(O)NHR₅, CH₂SC(O)NR₅R₆, CH₂NHCOR₅, CH₂NHC(O)OR₅, CH₂NHC(O)NH₂, CH₂NHC(O)NHR₅, CH₂NHC(O)NR₅R₆, CH₂N(R₆)COR₅, CH₂N(R₆)C(O)OR₅, CH₂N(R₆)C(O)NH₂, CH₂N(R₆)C(O)NHR₅, CH₂N(R₆)C(O)NR₅R₆, CH(OR₅)₂, CH(OH)OR₅, CO₂R₅, C(O)NH₂, C(O)NHR₅, C(O)NR₅R₆, C(O)NHOH, or C(O)NHOR₅;

R₄ is selected from CH₃, CH₂NH₂, CH₂OH, CH₂SH, CH₂SePh, CH₂X, CH₂NHOH, CN, CHO, CO₂H, CH₂NHOR₅, CH═N—OH, CH═N—OR₅, CH═N—OCOR₅, CH₂NHR₅, CH₂NH(CH₂)ₙX, CH₂NR₅R₆, CH₂OR₅, CH₂OCOR₅, CH₂OC(O)OR₅, CH₂OCO(CH₂)ₙX, CH₂OCO(HC═CH)ₙR₅, CH₂OC(O)NH₂, CH₂OC(O)NHR₅, CH₂OC(O)NR₅R₆, CH₂OSO₂(R₅), CH₂OSO₂(CH₂)ₙX, CH₂OSi(R₅)ₘ(R₆)₃₋ₘ, CH₂SR₅, CH₂SCOR₅, CH₂SCO(CH₂)ₙX, CH₂SC(O)NH₂, CH₂SC(O)NHR₅, CH₂SC(O)NR₅R₆, CH₂NHCOR₅, CH₂NHC(O)OR₅, CH₂NHC(O)NH₂, CH₂NHC(O)NHR₅, CH₂NHC(O)NR₅R₆, CH₂N(R₆)COR₅, CH₂N(R₆)C(O)OR₅, CH₂N(R₆)C(O)NH₂, CH₂N(R₆)C(O)NHR₅, CH₂N(R₆)C(O)NR₅R₆, CH(OR₅)₂, CH(OH)OR₅, CO₂R₅, C(O)NH₂, C(O)NHR₅, C(O)NR₅R₆, C(O)NHOH, or C(O)NHOR₅;

R₅ and R₆ are independently a straight or branched C₁₋₈ alkyl, aryl-C₁₋₈ alkyl, heterocycle-C₁₋₈ alkyl, cyclo(C₃₋₉)alkyl, aryl, or heterocycle, wherein

the aryl comprises phenyl or a polycyclic aryl group such as naphthyl;

the heterocyle comprises
a five or six membered aromatic heterocycle such as pyridyl, diazinyl, pyrimidinyl, 5-methoxy pyrimidinyl, (1,2,4)triazine-3,5-dione-6-yl, 6-mercaptopyrimidine-4-yl, pyrrolyl, pyrazolyl, imidazolyl, oxazolyl, isoxazolyl, thiazolyl, isothiazolyl, furanyl, or thienyl;
a 3 to 9 membered non-aromatic heterocycle comprising piperazinyl, 4-methyl piperazinyl, pyrrolidinyl, imidazolidinyl, imidazolinyl, thiazolidinyl, thiazolinyl, isothiazolidinyl, isothiazolinyl, pyranyl, or morpholinyl; or
a polycyclic heterocycle such as indolyl, benzthienyl, benzofuranyl, isoindolyl, isobenzothienyl, or isobenzofuranyl;

wherein each alkyl, cycloalkyl, aryl, and heterocycle are each optionally substituted with one or more groups (e.g., 1, 2, or 3 group) which are each independently C₁₋₈ alkyl, C₁₋₈ alkoxy, C₁₋₈ alkylamino, di(C₁₋₈ alkyl)amino, C₁₋₈ alkylamino-C₁₋₈ alkyl, di(C₁₋₆ alkyl)amino-C₁₋₈ alkyl, OC(O)OR₇, OC(O)N(R₇)₂, C(O)OR₇, C(O)N(R₇)₂, S(O)₂R₇, S(O)₂N(R₇)₂, N(R₇)C(O)N(R₇)₂, N(R₇)C(O)OR₇, CN, N₃, NHOH, ═NOH, NH₂, NO₂, OH, SH, F, Cl, Br, or I, wherein each R₇ is independently H or C₁₋₈ alkyl; or

R₅ and R₆ taken together with the carbon atoms to which they are attached form a cyclo(C₃₋₉)alkyl, aryl, or heterocycle, wherein

the aryl comprises a phenyl or a polycyclic aryl group;

the heterocyle comprises
a five or six membered aromatic heterocycle, such as pyridyl, diazinyl, pyrimidinyl, 5-methoxy pyrimidinyl, (1,2,4)triazine-3,5-dione-6-yl, 6-mercaptopyrimidine-4-yl, pyrrolyl, pyrazolyl, imidazolyl, oxazolyl, isoxazolyl, thiazolyl, isothiazolyl, furanyl, or thienyl;
a 3 to 9 membered non-aromatic heterocycle comprising piperazinyl, pyrrolidinyl, imidazolidinyl, imidazolinyl, thiazolidinyl, thiazolinyl, isothiazolidinyl, isothiazolinyl, 4-methyl piperazinyl, pyranyl, or morpholinyl; or
a polycyclic heterocycle such as indolyl, benzthienyl, benzofuranyl, isoindolyl, isobenzothienyl, or isobenzofuranyl;

wherein the cycloalkyl, aryl, and heterocycle are each optionally substituted with one or more groups (e.g., 1, 2, or 3 groups) which are each independently C₁₋₈ alkyl, C₁₋₈ alkoxy, C₁₋₈ alkylamino, di(C₁₋₈ alkyl)amino, C₁₋₈ alkylamino-C₁₋₈ alkyl, di(C₁₋₆ alkyl)amino-C₁₋₈ alkyl, OC(O)OR₇, OC(O)N(R₇)₂, C(O)OR₇, C(O)N(R₇)₂, S(O)₂R₇, S(O)₂N(R₇)₂, N(R₇)C(O)N(R₇)₂, N(R₇)C(O)OR₇, CN, N₃, NHOH, ═NOH, NH₂, NO₂, OH, SH, F, Cl, Br, or I wherein each R₇ is independently H or C₁₋₈ alkyl;

each X is independently Y or aryl, heterocycle, or cyclo(C₃₋₉)alkyl, wherein the aryl, heterocycle, or cyclo(C₃₋₉)alkyl are each optionally substituted with one or more Y groups (e.g., 1, 2, or 3 Y groups), wherein each Y is independently F, Cl, Br, I, CN, N₃, NHOH, ═NOH, NH₂, OH, SH, NHR₅, NR₅R₆, OR₅, SR₅, CO₂H, CO₂R₅, SO(OH)₂, or SO(OR₅)₂;

each m is independently 0, 1, 2, or 3;

each n is independently 1, 2, 3, 4, or 5;

with the proviso that:

when bond a is a single bond, bond b is a double bond, R₁ is α-OH, R₂ is CH₂OH, and R₃ is CO₂H, then R₄ is not CH₃;

when bond a is a single bond, bond b is a double bond, R₁ is α-OH, R₂ and R₃ are CO₂H or CO₂Me, then R₄ is not CH₃;

when bond a is a single bond, bond b is a double bond, R₁ is α-OCOCH₃, R₂ and R₃ are CO₂H, then R₄ is not CH₃;

when bond a is a single bond, bond b is a double bond, R₁ is β-OH, R₂ and R₃ are CO₂Me, then R₄ is not CH₃;

when bond a is a double bond, bond b is a double bond, R₁ is ═O, R₂ and R₃ are CO₂Me, then R₄ is not CH₃;

when bond a is a single bond, bond b is a double bond, R₁ is α-OH, R₂ and R₃ are CH₂OH, then R₄ is not CH₃;

when bond a is a single bond, bond b is a double bond, R₁ is β-OH, R₂ and R₃ are CH₂OH, then R₄ is not CH₃;

when bond a is a single bond, bond b is a double bond, R₁ is β-OH, R₃ is CO₂H, and R₄ is CH₃, then R₂ is not CHO;

when bond a is a single bond, bond b is a double bond, R₁ is β-OH, R₂ is CH₂OH, and R₄ is CH₃, then R₃ is not CHO, CO₂H, CH₂CH═CH₂, CH₂C≡CH, CH₂C≡CCH₃, CH₂CH₂OH, (CH₂)₄OH, or (CH₂)₆OH;

when bond a is a double bond, bond b is a double bond, R₁ is ═O, R₃ is CO₂H, and R₄ is CH₃, then R₂ is not CH₂OH, or CO₂H;

when bond a is a single bond, bond b is a double bond, R₁ is β-OCOCH₃, R₂ is CH₂OCOCH₃, and R₄ is CH₃, then R₃ is not CO₂H, CH₂CH₂OH, (CH₂)₄OH, or (CH₂)₆OH;

when bond a is a single bond, bond b is a double bond, R₁ is β-OCOCH₂CH₃, R₂ is CH₂OCOCH₂CH₃, and R₄ is CH₃, then R₃ is not CO₂H;

when bond a is a single bond, bond b is a double bond, R₁ is α-OSO₂Ph, R₂ and R₃ are CH₂OSO₂Ph, then R₄ is not CH₃;

when bond a is a single bond, bond b is a double bond, R₁ is β-OCO-4′-Cl-Ph, R₃ is CO₂H, and R₄ is CH₃, then R₂ is not CH₂OCO-4′-Cl-Ph; and

when bond a is a single bond, bond b is a double bond, R₁ is β-OCO-benzyl, R₃ is CO₂H, and R₄ is CH₃, then R₂ is not CH₂OCO-benzyl.

In one embodiment of the first aspect, a compound of formula I is provided wherein

when bond a is a double bond, R₁ is ═O, ═N—OH, or ═N—OCOCH₃; or when bond a is a single bond, R₁ is OH, NH₂, NHCOCH₃, OCOCH₃, or tetrahydro-2H-pyran-2-yloxy-;

R₂ is CH₂OH, CH₂OCOCH₃, or (tetrahydro-2H-pyran-2-yloxy)methyl-;

R₃ is CO₂H, CH₂OH, CH₂OCOCH₃, CH₂NH₂, CH₂NHCH₂CH₂OH, CN, tetrahydro-2H-pyran-2-yloxy)methyl-, CH═NOH, or CHO; and

R₄ is CH₃, CH₂OH, CH₂NHCH₂CH₂OH, CH₂OSO₂CH₃, CHO, or CO₂H.

In one aspect of this embodiment, a compound of formula I is provided wherein

R₁ is OH, ═O, ═N—OH, ═N—OCOCH₃, NH₂, NHCOCH₃, or OCOCH₃;

R₂ is CH₂OH or CH₂OCOCH₃;

R₃ is CO₂H, CH₂OH, CH₂OCOCH₃, CH₂NH₂, CH₂NHCH₂CH₂OH, CN, or CH═NOH; and

R₄ is CH₃, CH₂OH, or CHO.

In another embodiment of the first aspect, a compound of formula I is provided, wherein

R₁ is ═N—OCOCH₃; R₂ is CH₂OCOCH₃; R₃ is CO₂H; and R₄ is CH_{3 [}14];

R₁ is ═N—OCOCH₃; R₂ is CH₂OCOCH₃; R₃ is (tetrahydro-2H-pyran-2-yloxy)methyl-; and R₄ is CH₃;

R₁ is ═N—OCOCH₃; R₂ is CH₂OCOCH₃; R₃ is CH₂OH; and R₄ is CH₃;

R₁ is ═N—OCOCH₃; R₂ is CH₂OCOCH₃; R₃ is CH₂OCOCH₃; and R₄ is CH₃;

R₁ is ═N—OH; R₂ is CH₂OH; R₃ is CO₂H; and R₄ is CH₃;

R₁ is ═N—OH; R₂ is CH₂OH; R₃ is (tetrahydro-2H-pyran-2-yloxy)methyl-; and R₄ is CH₃;

R₁ is ═N—OH; R₂ is CH₂OH; R₃ is CH₂OH; and R₄ is CH₃;

R₁ is ═N—OH; R₂ is CH₂OH; R₃ is CH₂OCOCH₃; and R₄ is CH₃;

R₁ is ═O; R₂ is CH₂OH; R₃ is CH₂OH; and R₄ is CH₃;

R₁ is ═O; R₂ is CH₂OH; R₃ is CH₂OCOCH₃; and R₄ is CH₃;

R₁ is OH; R₂ is CH₂OH; R₃ is CH₂OCOCH₃; and R₄ is CH₃;

R₁ is tetrahydro-2H-pyran-2-yloxy-; R₂ is (tetrahydro-2H-pyran-2-yloxy)methyl-;

R₃ is CH₂OCOCH₃; and R₄ is CH₃;

R₁ is tetrahydro-2H-pyran-2-yloxy-; R₂ is (tetrahydro-2H-pyran-2-yloxy)methyl-;

R₃ is CH₂OH; and R₄ is CH₃;

R₁ is NH₂; R₂ is CH₂OH; R₃ is CO₂H; and R₄ is CH₃;

R₁ is NH₂; R₂ is CH₂OH; R₃ is CH₂OH; and R₄ is CH₃;

R₁ is NHCOCH₃; R₂ is CH₂OCOCH₃; R₃ is CO₂H; and R₄ is CH₃;

R₁ is NHCOCH₃; R₂ is CH₂OCOCH₃; R₃ is CH₂OCOCH₃; and R₄ is CH₃;

R₁ is NHCOCH₃; R₂ is CH₂OH; R₃ is CO₂H; and R₄ is CH₃;

R₁ is NHCOCH₃; R₂ is CH₂OH; R₃ is CH₂OH; and R₄ is CH₃;

R₁ is ═N—OCOCH₃; R₂ is CH₂OCOCH₃; R₃ is CHO; and R₄ is CH₃;

R₁ is ═N—OCOCH₃; R₂ is CH₂OCOCH₃; R₃ is CH═NOH; and R₄ is CH₃;

R₁ is ═N—OH; R₂ is CH₂OH; R₃ is CH═NOH; and R₄ is CH₃;

R₁ is NH₂; R₂ is CH₂OH; R₃ is CH₂NH₂; and R₄ is CH₃;

R₁ is ═N—OH; R₂ is CH₂OH; R₃ is CHO; and R₄ is CH₃;

R₁ is ═N—OH; R₂ is CH₂OH; R₃ is CH₂NHCH₂CH₂OH; and R₄ is CH₃;

R₁ is NH₂; R₂ is CH₂OH; R₃ is CH₂NHCH₂CH₂OH; and R₄ is CH₃;

R₁ is ═N—OCOCH₃; R₂ is CH₂OCOCH₃; R₃ is CN; and R₄ is CH₃;

R₁ is ═N—OH; R₂ is CH₂OH; R₃ is CN; and R₄ is CH₃;

R₁ is NH₂; R₂ is CH₂OH; R₃ is CN; and R₄ is CH₃;

R₁ is ═O; R₂ is CH₂OCOCH₃; R₃ is CH₂OCOCH₃; and R₄ is CH₃;

R₁ is ═N—OCOCH₃, R₂ is CH₂OCOCH₃, R₃ is CO₂H, and R₄ is CH₂OH;

R₁ is ═N—OCOCH₃, R₂ is CH₂OCOCH₃, R₃ is CH₂OH and R₄ is CH₂OH;

R₁ is ═N—OCOCH₃, R₂ is CH₂OCOCH₃, R₃ is CH₂OCOCH₃, and R₄ is CH₂OH;

R₁ is ═N—OCOCH₃, R₂ is CH₂OCOCH₃, R₃ is CN, and R₄ is CH₂OH;

R₁ is ═O, R₂ is CH₂OCOCH₃, R₃ is CH₂OCOCH₃, and R₄ is CH₂OH;

R₁ is ═N—OH, R₂ is CH₂OCOCH₃, R₃ is CO₂H, and R₄ is CH₂OH;

R₁ is ═N—OH, R₂ is CH₂OCOCH₃, R₃ is CH₂OH and R₄ is CH₂OH;

R₁ is ═N—OH, R₂ is CH₂OCOCH₃, R₃ is CH₂OCOCH₃, and R₄ is CH₂OH;

R₁ is ═N—OH, R₂ is CH₂OCOCH₃, R₃ is CN, and R₄ is CH₂OH;

R₁ is ═O, R₂ is CH₂OCOCH₃, R₃ is CO₂H, and R₄ is CH₂OH;

R₁ is ═O, R₂ is CH₂OCOCH₃, R₃ is CH₂OH and R₄ is CH₂OH;

R₁ is ═O, R₂ is CH₂OCOCH₃, R₃ is CH₂OCOCH₃, and R₄ is CH₂OH;

R₁ is ═O, R₂ is CH₂OCOCH₃, R₃ is CN, and R₄ is CH₂OH;

R₁ is OH, R₂ is CH₂OCOCH₃, R₃ is CO₂H, and R₄ is CH₂OH;

R₁ is OH, R₂ is CH₂OCOCH₃, R₃ is CH₂OH and R₄ is CH₂OH;

R₁ is OH, R₂ is CH₂OCOCH₃, R₃ is CH₂OCOCH₃, and R₄ is CH₂OH;

R₁ is OH, R₂ is CH₂OCOCH₃, R₃ is CN, and R₄ is CH₂OH;

R₁ is OH, R₂ is CH₂OCOCH₃, R₃ is CH₂OCOCH₃, and R₄ is CH₂OH;

R₁ is OCOCH₃, R₂ is CH₂OH, R₃ is CH₂OCOCH₃, and R₄ is CH₂OH;

R₁ is NH₂, R₂ is CH₂OCOCH₃, R₃ is CO₂H, and R₄ is CH₂OH;

R₁ is NH₂, R₂ is CH₂OCOCH₃, R₃ is CH₂OH and R₄ is CH₂OH;

R₁ is NH₂, R₂ is CH₂OCOCH₃, R₃ is CH₂OCOCH₃, and R₄ is CH₂OH;

R₁ is NH₂, R₂ is CH₂OCOCH₃, R₃ is CN, and R₄ is CH₂OH;

R₁ is ═N—OCOCH₃, R₂ is CH₂OCOCH₃, R₃ is CH₂OCOCH₃, and R₄ is CH₂OOSO₂CH₃;

R₁ is ═N—OCOCH₃, R₂ is CH₂OCOCH₃, R₃ is CN, and R₄ is CH₂O SO₂CH₃;

R₁ is ═N—OH, R₂ is CH₂OH, R₃ is CH₂OH, and R₄ is CH₂OOSO₂CH₃;

R₁ is ═N—OH, R₂ is CH₂OH, R₃ is CN, and R₄ is CH₂OOSO₂CH₃;

R₁ is ═N—OH, R₂ is CH₂OH, R₃ is CH₂OH, and R₄ is CH₂NHCH₂CH₂OH;

R₁ is ═N—OH, R₂ is CH₂OH, R₃ is CN, and R₄ is CH₂NHCH₂CH₂OH;

R₁ is NH₂, R₂ is CH₂OH, R₃ is CH₂OH, and R₄ is CH₂NHCH₂CH₂OH;

R₁ is NH₂, R₂ is CH₂OH, R₃ is CN, and R₄ is CH₂NHCH₂CH₂OH;

R₁ is ═O, R₂ is CH₂OCOCH₃, R₃ is CH₂OCOCH₃, and R₄ is CHO;

R₁ is ═O, R₂ is CH₂OCOCH₃, R₃ is CH₂OCOCH₃, and R₄ is CO₂H;

R₁ is ═O, R₂ is CH₂OH, R₃ is CH₂OH, and R₄ is CHO; or

R₁ is ═O, R₂ is CH₂OH, R₃ is CH₂OH, and R₄ is CO₂H.

In another aspect, a compound of formula I is provided wherein bond a is a double bond; bond b is a double bond; R₁ is ═O; R₂ is H; R₃ is CH₂OH; and R₄ is CHO.

In another aspect, the invention provides pharmaceutical compositions comprising a compound according to the first aspect of the invention and a pharmaceutically acceptable carrier, excipient, or diluent.

In yet another aspect, the invention provides methods for inhibiting cancer in a cell comprising contacting the cell in which inhibition is desired with an effective amount of a compound according to the first aspect of the invention and a pharmaceutically acceptable carrier, excipient, or diluent.

In still another aspect, the invention provides methods of treating a disease comprising administering to a patient a pharmaceutical composition comprising a compound according to the first aspect of the invention and a pharmaceutically acceptable carrier, excipient, or diluent. In one embodiment, the disease involves a cell proliferative condition. In another embodiment, the cell proliferative condition is cancer. In still another embodiment, the cancer is melanoma, glioblastoma, ovarian carcinoma, colon carcinoma, and breast carcinoma, or cervical cancer.

In another embodiment, the invention provides methods for inhibiting viruses, bacteria or malaria in a cell comprising contacting the cell in which inhibition is desired with an effective amount of a compound according to the first aspect of the invention and a pharmaceutically acceptable carrier, excipient, or diluent. In still another embodiment, the invention provides methods for treating inflammation comprising administering to a patient a pharmaceutical composition comprising a compound according to the first aspect of the invention and a pharmaceutically acceptable carrier, excipient, or diluent.

In yet another embodiment, the invention provides methods for synthesis of compounds according to the first aspect of the invention.

These and other aspects of the present invention will become apparent from the following description of the invention, which are intended to limit neither the spirit nor scope of the invention but are only offered as illustrations of the preferred embodiments of the invention.

### DETAILED DESCRIPTION OF THE INVENTION

The chemistries described in the invention represent methods for the syntheses of compounds of the general structural formula I and, therefore, the present invention relates to compounds, compositions and methods for preventing and treating diseases, for the prevention and inhibition of tumor growth and for the treatment of malignant tumors such as melanoma, glioblastoma, ovarian carcinoma, colon carcinoma, and breast carcinoma.

### DEFINITIONS

As used in the present specification, the following words and phrases are generally intended to have the meanings as set forth below, except to the extent that the context in which they are used indicates otherwise or they are expressly defined to mean something different.

The symbol “—” means a single bond, “═” means a double bond, “≡” means a triple bond, [symbol] means a single or double bond.

When chemical structures are depicted or described, unless explicitly stated otherwise, all carbons are assumed to have hydrogen substitution to conform to a valence of four. For example, in the structure on the left-hand side of the schematic below there are nine hydrogens implied. The nine hydrogens are depicted in the right-hand structure. Sometimes a particular atom in a structure is described in textual formula as having a hydrogen or hydrogens as substitution (expressly defined hydrogen), for example, —CH₂CH₂—. It is understood by one of ordinary skill in the art that the aforementioned descriptive techniques are common in the chemical arts to provide brevity and simplicity to description of otherwise complex structures.

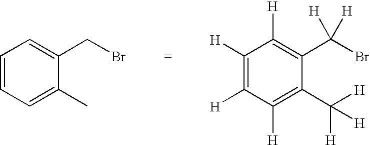

“Alkyl” is intended to include linear, branched, or cyclic hydrocarbon structures and combinations thereof, inclusively. For example, “C₆ alkyl” may refer to an n-hexyl, iso-hexyl, cyclobutylethyl, and the like. Lower alkyl refers to alkyl groups of from one to six carbon atoms. Examples of lower alkyl groups include methyl, ethyl, propyl, isopropyl, butyl, s-butyl, t-butyl, isobutyl, pentyl, hexyl and the like. Higher alkyl refers to alkyl groups containing more that eight carbon atoms. Exemplary alkyl groups are those of C₂₀ or below. Cycloalkyl is a subset of alkyl and includes cyclic hydrocarbon groups of from three to thirteen carbon atoms, as further defined below. In this application, alkyl refers to alkanyl, alkenyl, and alkynyl residues (and combinations thereof); it is intended to include cyclohexylmethyl, vinyl, allyl, isoprenyl, and the like. Thus when an alkyl residue having a specific number of carbons is named, all geometric isomers having that number of carbons are intended to be encompassed; thus, for example, either “butyl” or “C₄ alkyl” is meant to include n-butyl, sec-butyl, isobutyl, t-butyl, isobutenyl and but-2-ynyl groups; and for example, “propyl” or “C₃ alkyl” each include n-propyl, propenyl, and isopropyl. Alkyl also includes unsaturated hydrocarbon groups, such as alkenyl and alkynyl groups each having one or more carbon-carbon double or triple bonds, respectively.

“Alkoxy” or “alkoxyl” refers to the group —O-alkyl, for example including from one to eight carbon atoms of a straight, branched, cyclic configuration, unsaturated chains, and combinations thereof attached to the parent structure through an oxygen atom. Examples include methoxy, ethoxy, propoxy, isopropoxy, cyclopropyloxy, cyclohexyloxy and the like. Lower-alkoxy refers to groups containing one to six carbons.

“Aryl” refers to aromatic six- to fourteen-membered carbocyclic ring, and includes mono-, bicyclic or polycyclic groups, for example, benzene, naphthalene, acenaphthylene, anthracene, indane, tetralin, fluorene and the like. Aryl as substituents includes univalent or polyvalent substituents. As univalent substituents, the aforementioned ring examples are named, phenyl, naphthyl, acenaphthyl, anthracenyl, indanyl, tetralinyl, and fluorenyl. “Polycyclic aryl” as used herein refers to an aryl ring fused to at least a second aryl ring. Examples of polycyclic aryl include, but are not limited to, naphthyl, anthracenyl, acenaphthylenyl, and phenanthrenyl.

When a group is referred to as “arylalkyl”, such as “aryl-C₁-C₈ alkyl”, an aryl moiety is attached to a parent structure via an alkylene group. Examples include benzyl, phenethyl, and the like. Both the aryl and the corresponding alkylene portion of a “C₁-C₈ alkyl” group may be optionally substituted.

In some examples, as appreciated by one of ordinary skill in the art, two adjacent groups on an aromatic system may be fused together to form a ring structure. The fused ring structure may contain heteroatoms and may be optionally substituted with one or more groups. It should additionally be noted that saturated carbons of such fused groups (i.e. saturated ring structures) can contain two substitution groups.

“Cycloalkyl” refers to a “cycloalkanyl”, “cycloalkenyl”, and “cycloalkynyl” groups, where “cycloalkanyl” refers to fully saturated hydrocarbon rings; “cycloalkenyl” refers to non-aromatic hydrocarbon rings containing at least one carbon-carbon double bond; “cycloalkynyl” refers to non-aromatic hydrocarbon rings containing at least one carbon-carbon triple bond. Each cycloalkyl group can be a monocyclic, fused or bridged bicyclic, fused or bridged tricyclic, fused or bridged polycyclic hydrocarbon group comprising 3 to 14 carbon atoms in the cycloalkyl ring, where the cycloalkyl can be saturated or unsaturated with one or more carbon-carbon double and/or triple bonds between consecutive ring atoms. Examples of cycloalkanyl group include, but are not limited to, cyclopropyl, cyclobutyl, cyclopentyl, cyclohexyl, cyclooctyl, decahydronaphthalenyl, bicyclo[2.2.1]heptanyl, adamantyl, and bicyclo[2.2.2]octanyl. Examples of cycloalkenyl group include, but are not limited to, cyclopentenyl, cyclohexenyl, cyclooctenyl, cyclooctadienyl, octahydronaphthalenyl, norbornenyl, and bicyclo[2.2.2]octenyl. Examples of cycloalkynyl group include, but are not limited to, cyclooctynyl and cyclodecynyl.

“Fused-polycyclic” or “fused ring system” refers to a polycyclic ring system that contains bridged or fused rings; that is, where two rings have more than one shared atom in their ring structures. In this application, fused-polycyclics and fused ring systems are not necessarily all aromatic ring systems. Typically, but not necessarily, fused-polycyclics share a vicinal set of atoms, for example naphthalene or 1,2,3,4-tetrahydro-naphthalene. A spiro ring system is not a fused-polycyclic by this definition, but fused polycyclic ring systems of the invention may themselves have spiro rings attached thereto via a single ring atom of the fused-polycyclic.

“Halogen” or “halo” refers to fluorine, chlorine, bromine or iodine. “Haloalkyl” and “haloaryl” refer generically to alkyl and aryl groups that are substituted with one or more halogens, respectively. Thus, “dihaloaryl,” “dihaloalkyl,” “trihaloaryl,” etc. refer to aryl and alkyl substituted with a plurality of halogens, but not necessarily a plurality of the same halogen; thus 4-chloro-3-fluorophenyl is within the scope of dihaloaryl. The phrase “mono- to per-halogenated” when combined with another group refers to groups wherein one hydrogen, more than one hydrogen, or all hydrogens are replaced with a halo. For example, a “mono- to per-halogenated methyl” would encompass groups such as —CH₂F, —CHCl₂ or —CF₃.

“Heterocycle” or “heterocyclyl” refers to a stable three- to fifteen-membered ring substituent that consists of carbon atoms and from one to five heteroatoms selected from the group consisting of nitrogen, phosphorus, oxygen and sulfur. A heterocycle includes an aromatic heterocyclyl group. For purposes of this invention, the heterocyclyl substituent may be a monocyclic, bicyclic or tricyclic ring system, which may include fused or bridged ring systems as well as spirocyclic systems; and the nitrogen, phosphorus, carbon or sulfur atoms in the heterocyclyl group may be optionally oxidized to various oxidation states. In a specific example, the group —S(O)₀₋₂—, refers to —S— (sulfide), —S(O)— (sulfoxide), and —SO₂— (sulfone). For convenience, nitrogens, particularly but not exclusively, those defined as annular aromatic nitrogens, are meant to include their corresponding N-oxide form, although not explicitly defined as such in a particular example. Thus, for a compound of the invention having, for example, a pyridyl ring; the corresponding pyridyl-N-oxide is meant to be included as another compound of the invention. In addition, annular nitrogen atoms may be optionally quaternized; and the ring substituent may be partially or fully saturated or aromatic. Examples of heterocyclyl groups include, but are not limited to, azetidinyl, acridinyl, benzodioxolyl, benzodioxanyl, benzofuranyl, carbazoyl, cinnolinyl, dioxolanyl, indolizinyl, naphthyridinyl, perhydroazepinyl, phenazinyl, phenothiazinyl, phenoxazinyl, phthalazinyl, pteridinyl, purinyl, quinazolinyl, quinoxalinyl, quinolinyl, isoquinolinyl, tetrazoyl, tetrahydroisoquinolyl, piperidinyl, piperazinyl, 2-oxopiperazinyl, 2-oxopiperidinyl, 2-oxopyrrolidinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, dihydropyridinyl, tetrahydropyridinyl, pyridinyl, pyrazinyl, pyrimidinyl, pyridazinyl, oxazolyl, oxazolinyl, oxazolidinyl, triazolyl, isoxazolyl, isoxazolidinyl, morpholinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzoxazolyl, furyl, tetrahydrofuryl, tetrahydropyranyl, thienyl, benzothienyl, thiamorpholinyl, thiamorpholinyl sulfoxide, thiamorpholinyl sulfone, dioxaphospholanyl, and oxadiazolyl.

Preferred heterocyclyls include, but are not limited to, acridinyl, azocinyl, benzimidazolyl, benzofuranyl, benzothiophenyl, benzoxazolyl, benzthiazolyl, benztriazolyl, pyridotriazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,3,4-triazolyl, and xanthenyl.

“Polycyclic heterocycle” as used herein refers to a heterocycle fused to at least one other aryl or heterocyclyl ring, as defined herein. Examples of bicyclic heterocycles include, but are not limited to, indolyl, benzimidazolyl, benzofuranyl, benzothiophenyl, benzoxazolyl, benzthiazolyl, benztriazolyl, pyridotriazolyl, benzisoxazolyl, benzisothiazolyl, and carbazolyl,

When a group is referred to as “heterocyclylalkyl” such as “heterocyclyl-C₁-C₈ alkyl” a heterocycle moiety is attached to a parent structure via an alkylene group. Examples include pyrid-2-ylmethyl, morpholin-4-ylmethyl, piperidin-1-ylmethyl, and the like. Both the heterocycle and the corresponding alkylene portion of a “C₁-C₆ alkyl-heterocyclyl” group may be optionally substituted, as defined herein.

“Optional” or “optionally” means that the subsequently described event or circumstance may or may not occur, and that the description includes instances where said event or circumstance occurs and instances in which it does not. One of ordinary skill in the art would understand that with respect to any molecule described as containing one or more optional substituents, only sterically practical and/or synthetically feasible compounds are meant to be included. “Optionally substituted” refers to all subsequent modifiers in a term. So, for example, in the term “optionally substituted aryl-C₁₋₈ alkyl,” both the “C₁₋₈ alkyl” portion and the “aryl” portion of the molecule may or may not be substituted. A list of exemplary optional substitutions is presented below in the definition of “substituted.”

“Substituted” alkyl, aryl, and heterocyclyl, refer respectively to alkyl, aryl, and heterocyclyl, one or more (for example up to about five, in another example, up to about three) hydrogen atoms are replaced by a substituent independently selected from: alkyl (for example, fluoromethyl), aryl (for example, 4-hydroxyphenyl), arylalkyl (for example, 1-phenyl-ethyl), heterocyclylalkyl (for example, 1-pyridin-3-yl-ethyl), heterocyclyl (for example, 5-chloro-pyridin-3-yl or 1-methyl-piperidin-4-yl), alkoxy, alkylenedioxy (for example methylenedioxy), amino (for example, alkylamino and dialkylamino), amidino, aryloxy (for example, phenoxy), arylalkyloxy (for example, benzyloxy), carboxy (—CO₂H), carboalkoxy (that is, acyloxy or —OC(═O)R), carboxyalkyl (that is, esters or —CO₂R), carboxamido, benzyloxycarbonylamino (CBZ-amino), cyano, acyl, halogen, hydroxy, nitro, sulfanyl, sulfinyl, sulfonyl, thiol, halogen, hydroxy, oxo, carbamyl, acylamino, and sulfonamido. And each substituent of a substituted group is optionally substituted, but these optional substituents themselves are not further substituted. Thus, an optionally substituted moiety is one that may or may not have one or more substituents, and each of the substituents may or may not have one or more substituents.

Some of the compounds of the invention may have imino, amino, oxo or hydroxy substituents off aromatic heterocyclyl systems. For purposes of this disclosure, it is understood that such imino, amino, oxo or hydroxy substituents may exist in their corresponding tautomeric form, i.e., amino, imino, hydroxy or oxo, respectively.

Compounds of the invention are named according to systematic application of the nomenclature rules agreed upon by the International Union of Pure and Applied Chemistry (IUPAC), International Union of Biochemistry and Molecular Biology (IUBMB), and the Chemical Abstracts Service (CAS).

The compounds of the invention, or their pharmaceutically acceptable salts, may have asymmetric carbon atoms, oxidized sulfur atoms or quaternized nitrogen atoms in their structure.

The compounds of the invention and their pharmaceutically acceptable salts may exist as any and all possible stereoisomers, geometric isomers, enantiomers, diastereomers and anomers. All such single stereoisomers, racemates and mixtures thereof, and geometric isomers are intended to be within the scope of this invention.

The description of the invention herein should be construed in congruity with the laws and principals of chemical bonding. It is assumed that when considering generic descriptions of compounds of the invention for the purpose of constructing a compound, such construction results in the creation of a stable structure. That is, one of ordinary skill in the art would recognize that theoretically some constructs which would not normally be considered as stable compounds (that is, sterically practical and/or synthetically feasible, supra).

When a particular group with its bonding structure is denoted as being bonded to two partners; that is, a divalent group, for example, —OCH₂—, then it is understood that either of the two partners may be bound to the particular group at one end, and the other partner is necessarily bound to the other end of the particular group, unless stated explicitly otherwise. Stated another way, divalent groups are not to be construed as limited to the depicted orientation, for example “—OCH₂—” is meant to mean not only “—OCH₂—” as drawn, but also “—CH₂O—.”

In addition to the preferred embodiments recited hereinabove, also preferred are embodiments comprising combinations of preferred embodiments.

Methods for the preparation and/or separation and isolation of single stereoisomers from racemic mixtures or non-racemic mixtures of stereoisomers are well known in the art. For example, optically active (R)- and (S)-isomers may be prepared using chiral synthons or chiral reagents, or resolved using conventional techniques. Enantiomers (R- and S-isomers) may be resolved by methods known to one of ordinary skill in the art, for example by: formation of diastereoisomeric salts or complexes which may be separated, for example, by crystallization; via formation of diastereoisomeric derivatives which may be separated, for example, by crystallization, selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where a desired enantiomer is converted into another chemical entity by one of the separation procedures described above, a further step may be required to liberate the desired enantiomeric form. Alternatively, specific enantiomer may be synthesized by asymmetric synthesis using optically active reagents, substrates, catalysts or solvents or by converting on enantiomer to the other by asymmetric transformation. For a mixture of enantiomers, enriched in a particular enantiomer, the major component enantiomer may be further enriched (with concomitant loss in yield) by recrystallization.

“Patient” for the purposes of the present invention includes humans and other animals, particularly mammals, and other organisms. Thus the methods are applicable to both human therapy and veterinary applications. In a preferred embodiment the patient is a mammal, and in a most preferred embodiment the patient is human.

“Therapeutically effective amount” is an amount of a compound of the invention, that when administered to a patient, ameliorates a symptom of the disease. The amount of a compound of the invention which constitutes a “therapeutically effective amount” will vary depending on the compound, the disease state and its severity, the age of the patient to be treated, and the like. The therapeutically effective amount can be determined routinely by one of ordinary skill in the art having regard to their knowledge and to this disclosure.

“Cancer” refers to cellular-proliferative disease states, including but not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hanlartoma, inesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis defornians), meninges (meningioma, meningiosarcoma, gliomatosis), brain (astrocytoma, medulloblastoma, glioma, ependymoma, germinoma [pinealoma], glioblastorna multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, SertoliLeydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma], fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles, dysplastic nevi, lipoma, angioma, dermatofibroma, keloids, psoriasis; and Adrenal glands: neuroblastoma. Thus, the term “cancerous cell” as provided herein, includes a cell afflicted by any one of the above-identified conditions.

“Bacterial infection” refers to diseases and conditions caused by Gram-positive and Gram-negative aerobic and anaerobic bacteria, including but not limited to: Streptococcus pneumoniae, Streptococcus pyogenes, Streptococcus agalactiae, Staphylococcus aureus, Staphylococcus epidermidis, Enterococcus faecalis, Enterococcus faecium, Escherichia coli, Enterobacter cloacae, Klebsiella pneumoniae, Proteus vulgaris, Pseudomonas aeruginosa, Acinetobacter baumannii, Haemophilus influenzae, Bacteroides fragilis, Bacteroides thetaiotaomicron, Porphyromonas asaccharolyticus, Prevotella melaminogenicus, Eubacterium lentum, Peptostrepococcus micros, Clostridium difficile, Clostridium perfringens, Mycobacterium tuberculosis, Mycobacterium avium complex, Helicobacter pyroli, Bacillus anthracis, Burkholderia mallei, Burkholderia cepacia, Burkholderia cepacia complex, Burkholderia pseudomallei, Francisella tularensis, and Yersinia pestis.

“Viral infection” refers to diseases and conditions caused by DNA viruses, RNA viruses and retroviruses, including but not limited to: herpes viruses, influenza viruses, arboviruses, varicella viruses, cytomegaloviruse, hepatitis A, B and C viruses, human immunodeficiency virus (HIV), bovine immunodeficiency virus, simian immunodeficiency virus, human T-cell leukemia viruses, feline leukemia virus, murine leumemia virus, avian sarcoma viruses (such as rous sarcoma virus), anemia virus, adenovirus, coronavirus, Epstein-Barr virus, human papillomavirus, Rhinovirus, Respiratory syncytial viruses, measles, mumps and rubella viruses.

“Pharmaceutically acceptable salt” include acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid, and the like, as well as organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

“Pharmaceutically acceptable base addition salts” include those derived from inorganic bases such as sodium, potassium, lithium, ammonium, calcium, magnesium, iron, zinc, copper, manganese, aluminum salts and the like. Exemplary salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. (See, for example, S. M. Berge, et al., “Pharmaceutical Salts,” J. Pharm. Sci., 1977; 66:1-19 which is incorporated herein by reference.)

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

In addition, it is intended that the present invention cover compounds made either using standard organic synthetic techniques, including combinatorial chemistry or by biological methods, such as bacterial digestion, metabolism, enzymatic conversion, and the like.

“Treating” or “treatment” as used herein covers the treatment of a disease-state in a human, which disease-state is characterized by abnormal cellular proliferation, and invasion and includes at least one of: (i) preventing the disease-state from occurring in a human, in particular, when such human is predisposed to the disease-state but has not yet been diagnosed as having it; (ii) inhibiting the disease-state, i.e., arresting its development; and (iii) relieving the disease-state, i.e., causing regression of the disease-state. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art.

### General Administration

In the second aspect, the invention provides pharmaceutical compositions comprising compounds according to the first aspect of the invention and a pharmaceutically acceptable carrier, excipient, or diluent. In certain other preferred embodiments, administration may preferably be by the oral route. Administration of the compounds of the invention, or their pharmaceutically acceptable salts, in pure form or in an appropriate pharmaceutical composition, can be carried out via any of the accepted modes of administration or agents for serving similar utilities. Thus, administration can be, for example, orally, nasally, parenterally (intravenous, intramuscular, or subcutaneous), topically, transdermally, intravaginally, intravesically, intracistemally, rectally, or via urethral, ocular intratumoral and irrigation method, in the form of solid, semi-solid, lyophilized powder, or liquid dosage forms, such as for example, tablets, suppositories, pills, soft elastic and hard gelatin capsules, powders, solutions, suspensions, or aerosols, or the like, preferably in unit dosage forms suitable for simple administration of precise dosages.

The compositions will include a conventional pharmaceutical carrier or excipient and a compound of the invention as the/an active agent, and, in addition, may include other medicinal agents, pharmaceutical agents, carriers, adjuvants, etc. Compositions of the invention may be used in combination with anticancer or other agents that are generally administered to a patient being treated for cancer. Adjuvants include preserving, wetting, suspending, sweetening, flavoring, perfuming, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

If desired, a pharmaceutical composition of the invention may also contain minor amounts of auxiliary substances such as wetting or emulsifying agents, pH buffering agents, antioxidants, and the like, such as, for example, citric acid, sorbitan monolaurate, triethanolamine oleate, butylalted hydroxytoluene, etc. The dosage form can be designed as a sustained release or timed release.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), dextrose, mannitol, polyvinylpyrrolidone, gelatin, hydroxycellulose, acacia, suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants. The liquid formulation can be buffered, isotonic solution.

One preferable route of administration is oral, using a convenient daily dosage regimen that can be adjusted according to the degree of severity of the disease-state to be treated.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate and the like, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid dosage forms as described above can be prepared with coatings and shells, such as enteric coatings and others well known in the art. They may contain pacifying agents, and can also be of such composition that they release the active compound or compounds in a certain part of the intestinal tract in a delayed manner. Examples of embedded compositions that can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., a compound(s) of the invention, or a pharmaceutically acceptable salt thereof, and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are, for example, suppositories that can be prepared by mixing the compounds of the present invention with for example suitable non-irritating excipients or carriers such as cocoa butter, polyethyleneglycol or a suppository wax, which are solid at ordinary temperatures but liquid at body temperature and therefore, melt while in a suitable body cavity and release the active component therein.

Dosage forms for topical administration of a compound of this invention include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this invention.

Generally, depending on the intended mode of administration, the pharmaceutically acceptable compositions will contain about 1% to about 99% by weight of a compound(s) of the invention, or a pharmaceutically acceptable salt thereof, and 99% to 1% by weight of a suitable pharmaceutical excipient. In one example, the composition will be between about 5% and about 75% by weight of a compound(s) of the invention, or a pharmaceutically acceptable salt thereof, with the rest being suitable pharmaceutical excipients.

Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of the invention, or a pharmaceutically acceptable salt thereof, for treatment of a disease-state in accordance with the teachings of this invention.

The compounds of the invention, or their pharmaceutically acceptable salts, are administered in a therapeutically effective amount which will vary depending upon a variety of factors including the activity of the specific compound employed, the metabolic stability and length of action of the compound, the age, body weight, general health, sex, diet, mode and time of administration, rate of excretion, drug combination, the severity of the particular disease-states, and the host undergoing therapy. The compounds of the present invention can be administered to a patient at dosage levels in the range of about 0.1 to about 1,000 mg per day. For a normal human adult having a body weight of about 70 kilograms, a dosage in the range of about 0.01 to about 100 mg per kilogram of body weight per day is an example. The specific dosage used, however, can vary. For example, the dosage can depend on a number of factors including the requirements of the patient, the severity of the condition being treated, and the pharmacological activity of the compound being used. The determination of optimum dosages for a particular patient is well known to one of ordinary skill in the art.

### Synthetic Procedures

The invention described above is illustrated by the examples outlined in the following reaction schemes without limiting the scope of the invention.

The corner stone reaction to hydroxylate lupane triterpenoids at the 23-position is the Baldwin's cyclopalladation of lupanone oxime.¹⁵ Thus, stereoselective cyclopalladation of oxime 10 and 11, respectively, prepared from the corresponding betulinic acid and betulin, at the C23 position with sodium tetrachloropalladate (II) in ethanol provided isolable dimeric organopalladium complex intermediates (12 and 13), which then underwent one-pot, sequential transformations to deliver 23-acetoxy derivatives (14 and 15): acylation of the oxime by acetic anhydride, dissociation of the palladium dimmer by pyridine to form monomeric pyridinium organiopalladium complex, 23-oxidation with Pb(OAc)₄ and reductive work up with NaBH₄ to remove palladium (Scheme 1). It is interesting to note that, along with 15a, partial replacement of the THP by acyl protecting group was observed under acidic conditions for oxidation, resulting in the formation of 28-hydroxy diacetate 15b and triacetate 15c, respectively. Jones oxidation of 15b yielded 14.

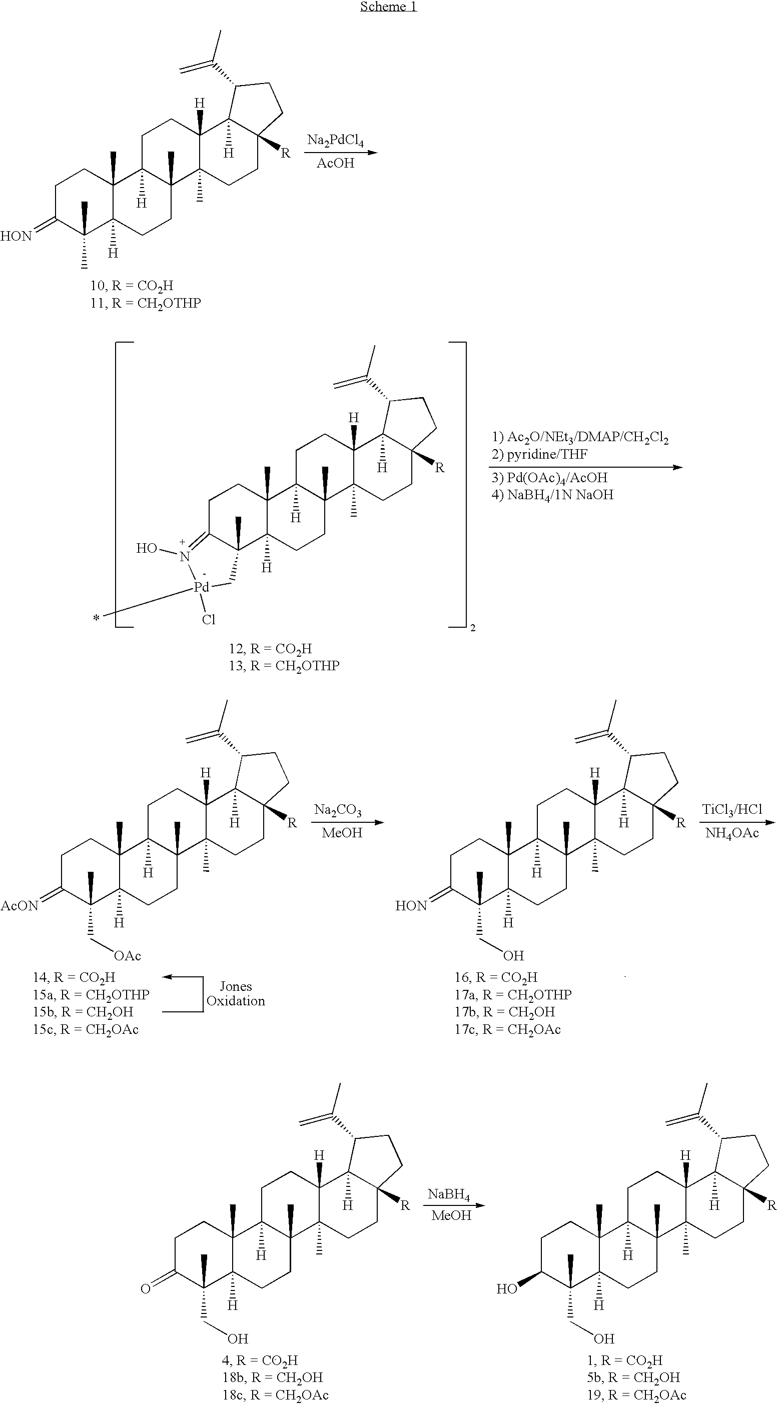

Removal of acetyl groups in 14 and 15 was achieved by methanolysis under the basic conditions (Na₂CO₃) to form 16 and 17. Partial methanolysis of 15c resulted in 17c, which can be further methanolyzed to 17b. The THP and acetyl groups in 15a can be removed in one step by PPTS and p-TsOH to afford 17b. Titanium nitride (TiN)-mediated deoximation of 16 and 17 using TiCl₃ and NH₄OAc led to the formation of 23-hydroxybetulonic acid (4) and the ketone derivatives 18 (Scheme 1). The THP group in 17a did not survive the deoximation condition. Reduction of 4 and 18 with NaBH₄ furnished 23-hydroxybetulinic acid (1), 23-hydroxybetulin (5b), and the acetate 19, respectively.

Swinniol (6) can be synthesized from 19 following the chemistry shown in Scheme 2.

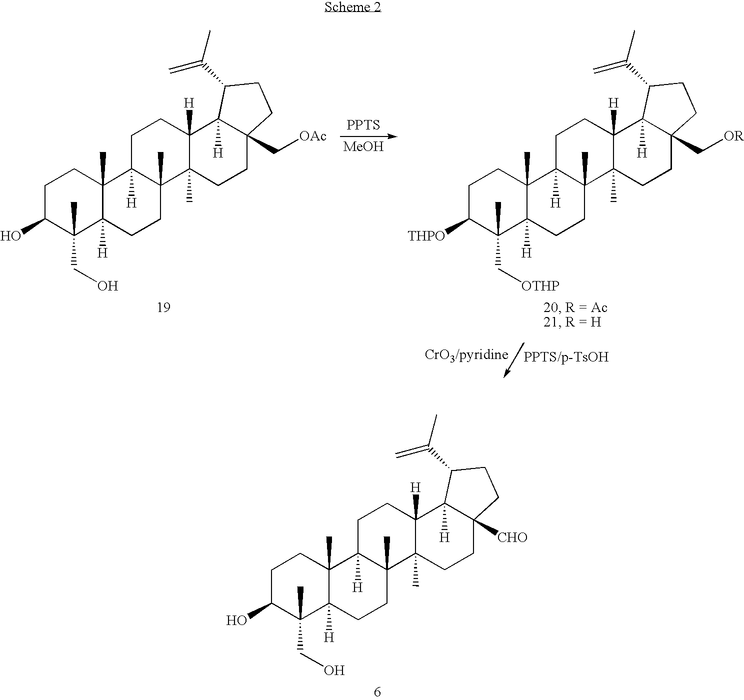

Further transformation of the functional groups in the 23-hydroxylated derivatives leads to the synthesis of novel 23-substituted lupane-type triterpenoids. For example, titanium nitride (TiN)-mediated reduction of 16 and 17b by NaCNBH₃ in the presence of TiCl₃ and NH₄OAc in the presence of NaCNBH₃ led to the formation of 3-amino derivatives 22 and 23 (Scheme 3). Acetylation of 22 and 23, followed by selective O-deacetylation resulted in N-Acetyl-3-amino compounds 26 and 27.

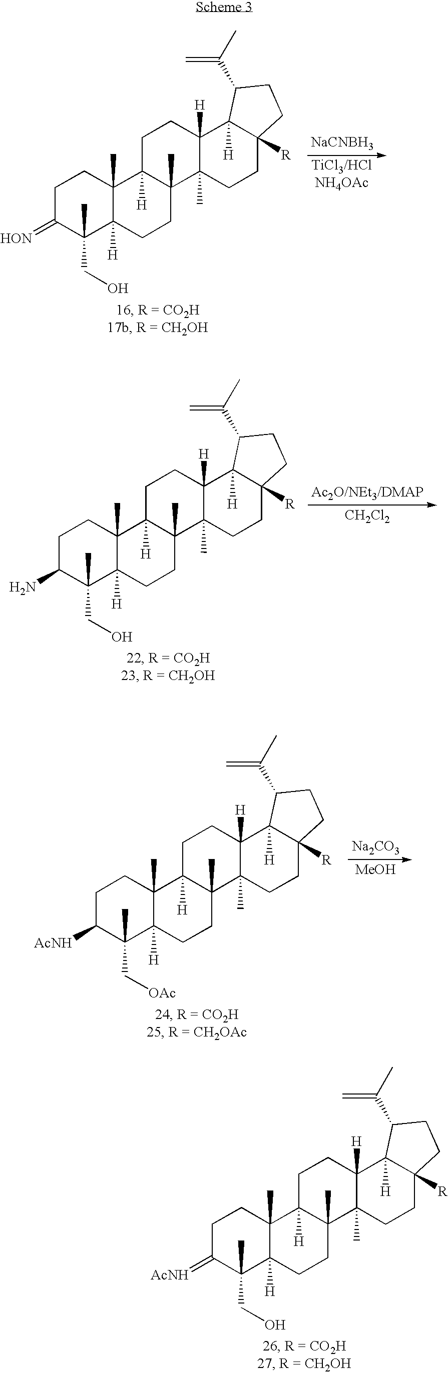

Access to the primary 3,28-bis-amino-23-hydroxy pentacyclic triterpenoid 31 is achieved efficiently according to the approach outlined in Scheme 4 from 15b. Thus, oxidation of the 28-OH in 3-acetoxime-23-acetoxy betulone 15b by CrO₃ under basic conditions furnished the corresponding aldehyde 28, which was converted to the bis(oxime) 29. Subsequent deacetylation by methanolysis under mild basic conditions and titanium nitride (TiN)-mediated reduction by NaCNBH₃ in the presence of TiCl₃/NH₄OAc yielded 23-hydroxy-3,28-bis-amino derivatives 31.

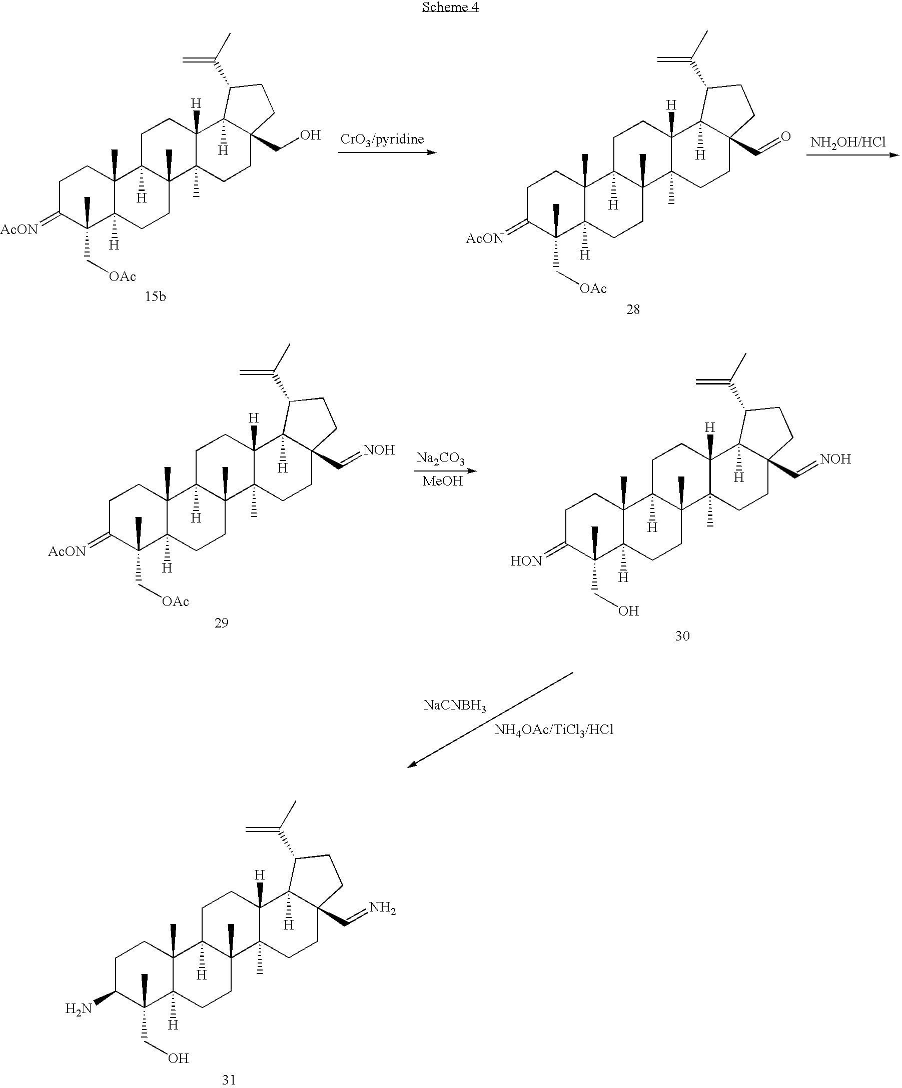

Construction of secondary amines at the C-28 position can be accomplished by standard reductive amination protocols as described in Scheme 5 from corresponding aldehyde. In order to prevent acyl transfer reactions during reductive amination step, the acetyl groups in aldehyde 28 were removed under mild methanolysis conditions to afford 32. Reductive amination on 32 with various amines in the presence of NaCNBH₃ successfully furnished the oxime-amino compounds 33, which then underwent titanium nitride (TiN)-mediated reduction by TiCl₃, NH₄OAc and NaCNBH₃ to deliver the targeted 3,28-bis(amino) analogues 34 in moderate yields.

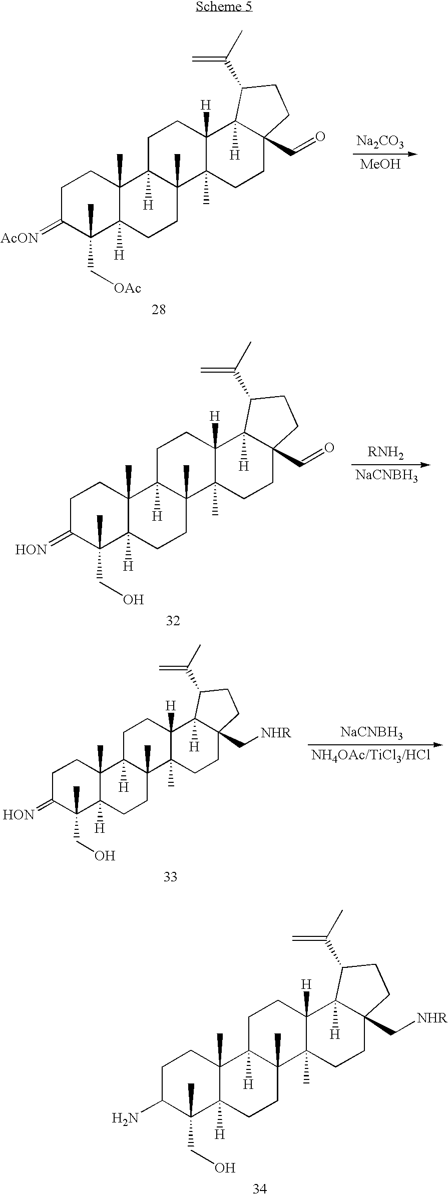

The 28-oxime group in 29 can undergo elimination under acetylation conditions (Ac₂O/pyridine) to form a nitrile 35 (Scheme 6). The 3-oxime in 35 was then converted to 3-amino derivative 37 following the chemistries described above.

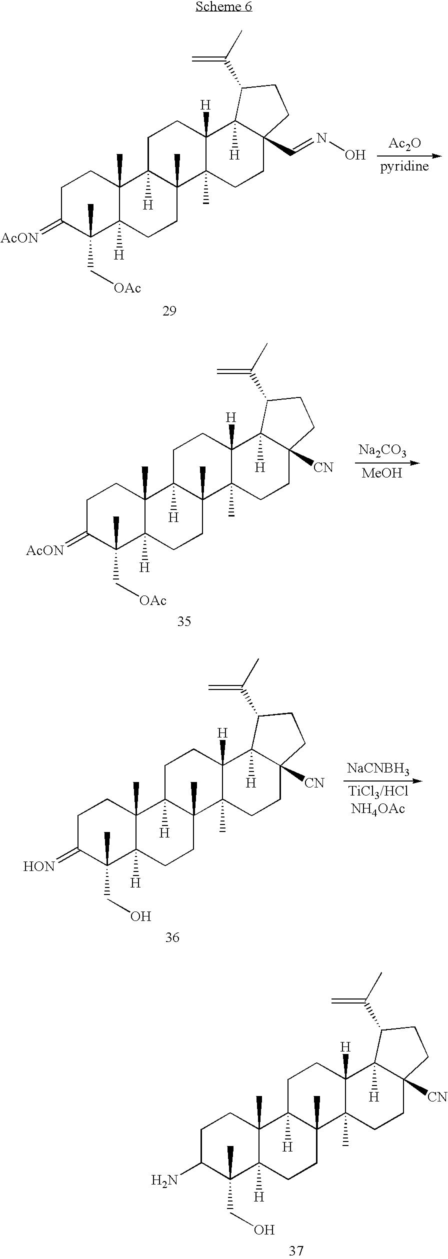

23-Hydroxy pentacyclic triterpenoids can also be modified at C-30 position to afford highly functionalized derivatives. The chemistry to introduce 30-hydroxy group onto 23-acetyloxy oximes or ketones, such as 14, 15b, 15c, 35 and 38, is shown in Scheme 7, which includes allylic selenation with PhSeCl and pyridine, followed by oxidation with 30% H₂O₂. 23-Acetoxy ketone 38 was obtained from 18b and 18c by acylation with Ac₂O/pyridine. The 30-OH-23-acetoxy oximes and ketones (39 and 40) obtained from allylic selenations can undergo further transformation. Thus, deacetylation of 39 and 40 by methanolysis in the presence of Na₂CO₃ resulted in the formation of 41 and 42, respectively (Scheme 7). Titanium nitride (TiN)-mediated deoximation of 41 by TiCl₃ and NH₄OAc led to the formation of 3-ketone derivatives 42a-d. Reduction of 42a-d by NaBH₄ afforded the 3-OH derivatives 43a-d, whereas, when the acetylated ketone 40c was reduced by NaBH₄, it appeared that acetyl exchange took place between 3- and 23-position with both 43c and 43f isolated, in addition to the expected product 43e. Deacetylation from 43c, 43e and 43f by methanolysis in the presence of Na₂CO₃ yielded the same tetraol 43b (Scheme 7).

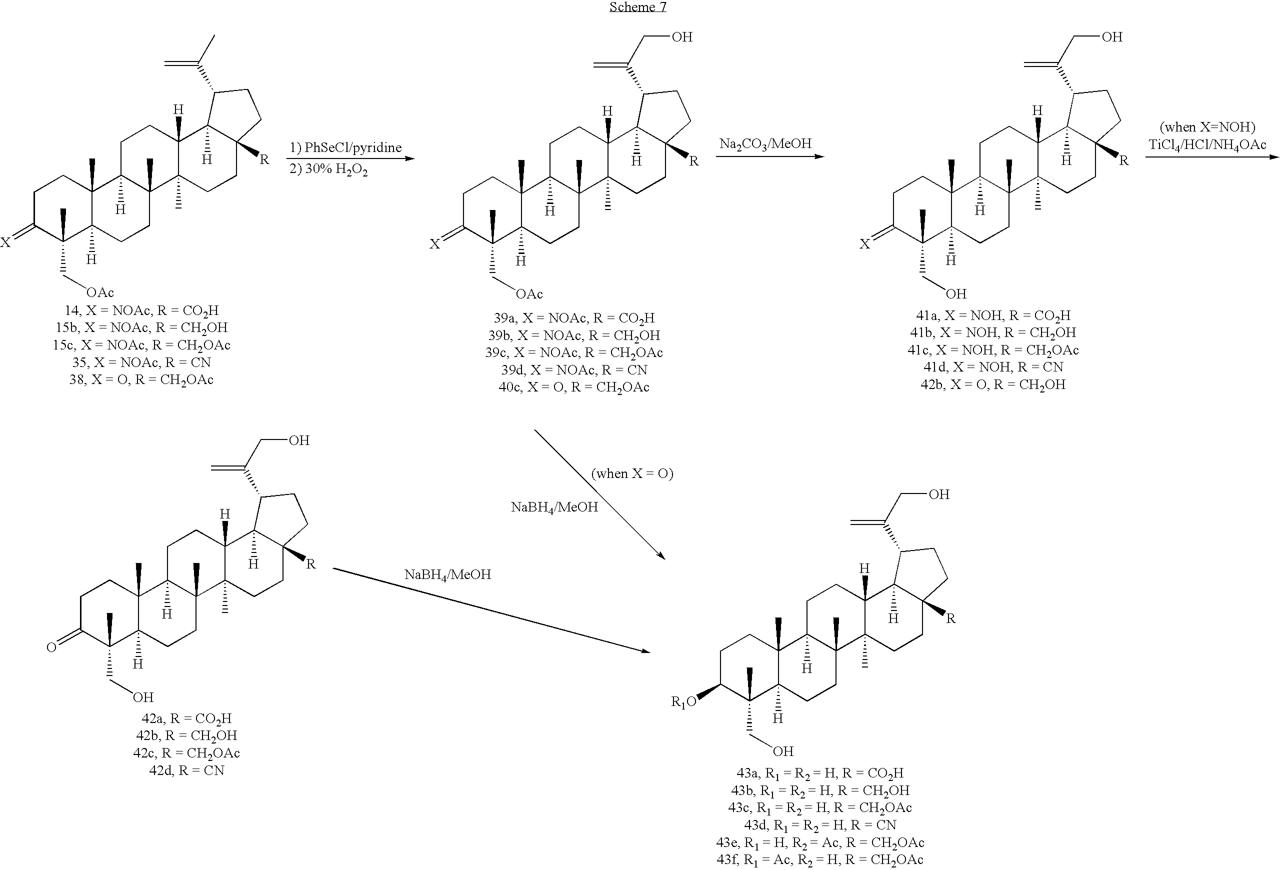

Titanium nitride (TiN)-mediated reduction of the corresponding 23,30-dihydroxy-3-oxime 41 by TiCl₃/NH₄OAc and NaCNBH₃ provided an excellent route to the novel 3-amino-23,30-dihydroxy pentacyclic triterpenoids 44 (Scheme 8).

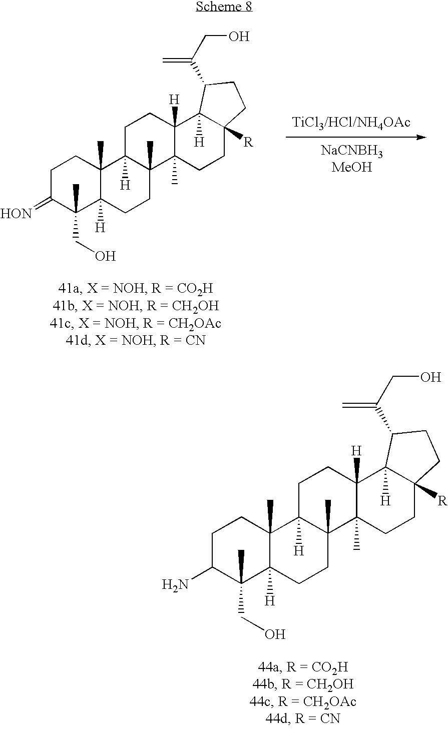

An amino group can be introduced to the 30-position by nucleophilic displacement of a leaving group with an appropriate amine. Thus, the mesyl group was assembled at the 30-position of 39 (Scheme 9). In order to prevent thermodynamically favorable acyl transfer reactions with the incoming amines in the next displacement step, acyl protecting groups in 45 were removed. The deprotected mesylate 46 underwent nucleophilic substitution with amines to yield 23-hydroxy-30-amino oxime 47, which was, in turn, reduced according to the TiCl₃/NH₄OAc/NaCNBH₃ reduction protocol as shown above to furnish the 23-hydroxy-3,30-bis(amino)-pentacyclic triterpenoids 48 (Scheme 9).

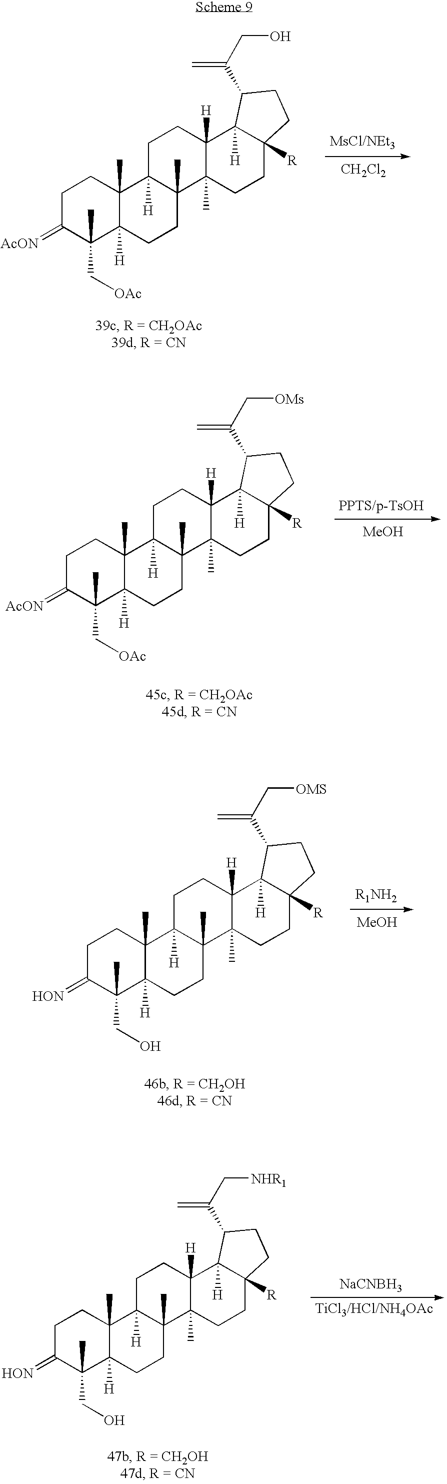

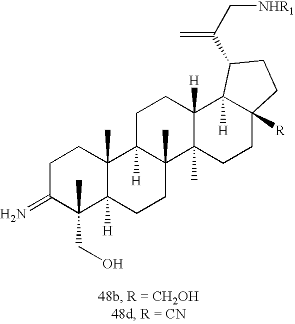

The 30-OH can be further oxidized to aldehyde or carboxylic acid, to prepare a new class of 23-hydroxy pentacyclic triterpenoids. For example, oxidation of partially protected keto triol 40c under basic conditions provided keto aldehyde 49a while Jone's oxidation generated the corresponding carboxylic acid 49b. Deacetylation of 49 by Na₂CO₃ in methanol freed the hydroxyl groups at both 23- and 28-positions to give 50, along with an unexpected 23-nor-hydroxyl aldehyde 51 due to a retro-aldol reaction to eliminate formaldehyde from 49b (Scheme 10).

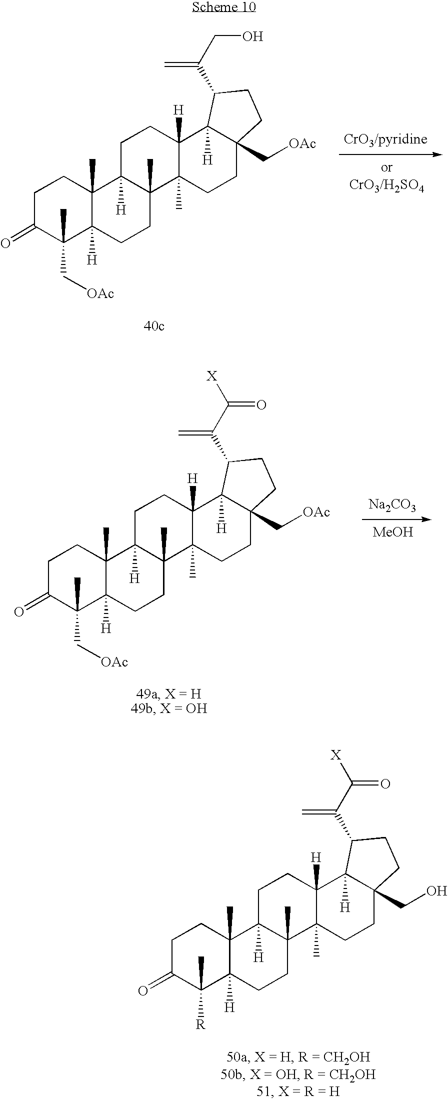

### EXPERIMENTAL SECTION

### Example 1

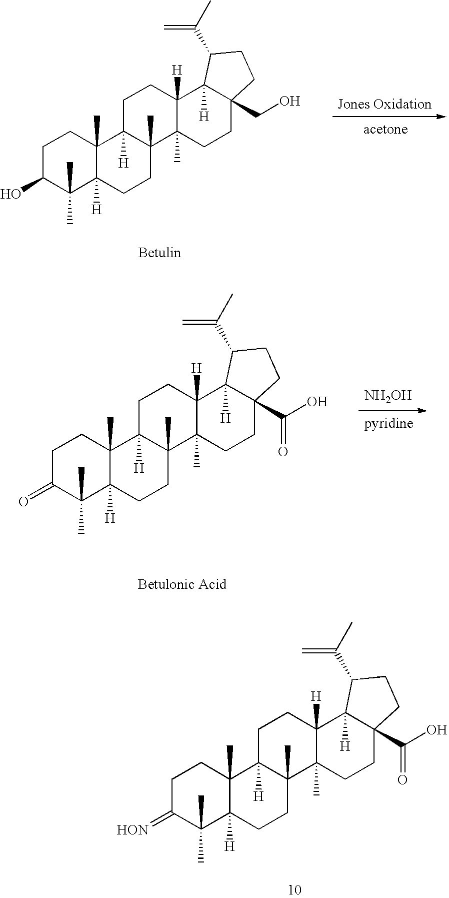

To a stirred suspension of betulin (5.0 g, 11.29 mmol) in acetone (150 mL) at 0° C. under N₂ was added dropwise freshly prepared Jones' reagent (19.5 mL, 1.96 M, 38.4 mmol). After 1 h, the ice bath was removed and stirring was continued at rt for 2 h, whereupon the reaction was washed with aqueous sodium metabisulfite solution (3.0 g in 200 mL of H₂O) and the aqueous layer was extracted with EtOAc (200 mL). The combined organic layers was dried (Na₂SO₄) and concentrated under reduced pressure to a crude material which was purified by silica gel gel flash column chromatography eluting with gradient 0-20% EtOAc in hexanes to furnish 2.9 g (57% yield) of betulonic acid as white solid, along with 0.29 g of betulone aldehyde.

To a stirred solution of betulonic acid (1.5 g, 3.30 mmol) and pyridine (1.0 mL, 13.2 mmol) in absolute EtOH (50 mL) at rt was added NH₂OH hydrochloride (255 mg, 3.96 mmol) all at once. After stirring at rt for 16 days, the solution was concentrated to about 10 mL, diluted with EtOAc (100 mL) and washed with brine (100 mL). The organic layer was dried (Na₂SO₄) and concentrated under reduced pressure to afford 1.6 g of oxime 10 as a foamy solid, which was used without further purification.

### Example 2

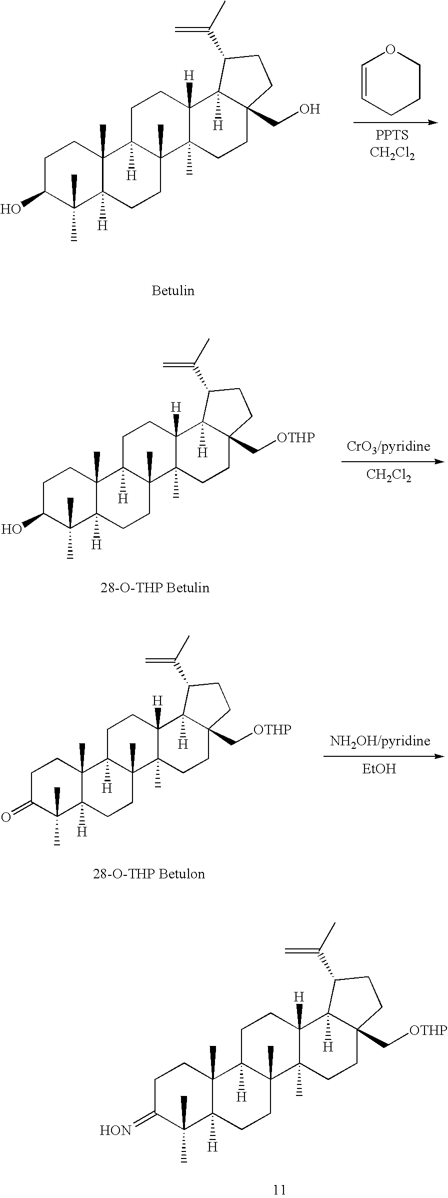

Into a stirring suspension of betulin (51.8 g, 0.117 mol) in anhydrous CH₂Cl₂ (1.5 L) at rt under N₂ was added 3,4-dihydro-2H-pyran (10.82 g, 0.128 mol) dropwise. After the addition was completed, pyridinium p-toluenesulfonic acid (PPTS) (3.45 g, 13.73 mmol) was added all at once. Stirring was continued at rt under N₂ for 2 weeks whereupon the reaction mixture was concentrated down to a volume of 500 mL at 40° C. and the mixture was washed with saturated NaHCO₃ (500 mL) and brine (500 mL). The organic solution was dried (Na₂SO₄) and concentrated under reduced pressure to a crude light yellow solid (60 g), which was purified by silica gel column chromatography eluting with gradient 5-50% EtOAc in hexanes to afford 36 g (58% yield) of desired 28-O-THP-betulin, along with 12.5 g (17% yield) of 3,28-bis(O-THP)-betulin and 14.2 g of recovered betulin.

To a stirred solution of pyridine (33 mL, 0.41 mol) in anhydrous CH₂Cl₂ (400 mL) at rt under N₂ was added CrO₃ (20.3 g, 0.20 mol) all at once. The resulting dark brown suspension was stirred at rt for 1 h, and then cooled to 0° C., into which a solution of 28-O-THP-betulin (18.0 g, 34.2 mmol) in anhydrous CH₂Cl₂ (100 mL) was added. The suspension was stirred at 0° C. for an additional hour and filtered through a fritted glass funnel containing silica gel (170 g). The solids were washed with additional CH₂Cl₂ (500 mL) and EtOAc (1 L). The combined filtrates were concentrated under reduced pressure to a light brown foam, which was purified by silica gel column chromatography eluting with gradient 0-20% EtOAc in hexanes as eluent to afford 17.3 g (96% yield) of 28-O-THP-betulon as white foam: m.p. 85-90° C.; ¹H NMR (400 MHz, CDCl₃) δ 0.8-2.1 (m), 0.92 (s), 0.98 (s), 1.02 (s), 1.05 (s), 1.07 (s), 1.68 (s), 2.44 (m), 2.99 (d, J=9.6 Hz), 3.38 (d, J=9.2 Hz), 3.53 (m), 3.85 (m), 3.93 (d, J=8.4 Hz), 4.57 (m), 4.68 (s).

To a stirred solution of 28-O-THP-betulon (1.0 g, 1.91 mmol) and pyridine (0.39 mL, 4.78 mmol) in absolute EtOH (50 mL) at rt was added NH₂OH hydrochloride (159 mg, 2.28 mmol) all at once. After stirring at rt for 16 h, the homogeneous solution was diluted with EtOAc (100 mL) and washed with H₂O (100 mL). The organic layer was separated, dried (Na₂SO₄) and concentrated under reduced pressure to a foamy solid which was purified by silica gel column chromatography eluting with gradient 5-20% EtOAc in hexanes to provide 500 mg (50% yield) of the oxime 11 as a white solid. ¹H NMR (500 MHz, CDCl₃) δ 0.90-2.10 (m), 0.91 (s), 0.96 (s), 1.03 (s), 1.04 (s), 1.06 (s), 1.13 (s), 1.68 (s), 2.24 (m), 2.44 (m), 2.95 (m), 2.98 (d, J=9.5 Hz), 3.37 (d, J=9.5 Hz), 3.52 (m), 3.85 (m), 3.92 (d, J=8.5 Hz), 4.56 (m), 4.67 (m), 7.59 (bs).

### Example 3

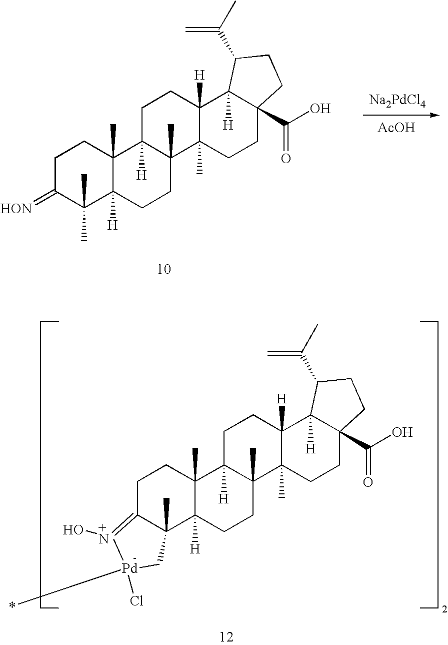

A suspension of oxime 10 (1.6 g, 3.41 mmol), Na₂PdCl₄ (1.1 g, 3.74 mmol), and NaOAc (308 mg, 3.74 mmol) in AcOH (100 mL) was stirred at rt for 3 days, whereupon it was poured onto ice (300 mL) and allowed to stand at rt for 2 h until all the ice was melted. The heterogeneous mixture was filtered and the collected solids were washed with additional water (50 mL) and allowed to air dry for 2 h. The yellow solids were dried further by addition of CH₂Cl₂ (200 mL) and concentration under reduced pressure at 60° C. to afford 1.8 g (90%) of the dimeric organopalladium complex 12 as light brown powder, which was taken to the next step without further purification.

### Example 4

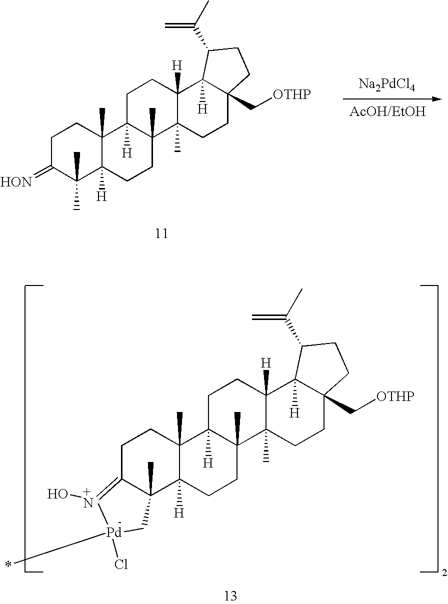

A suspension of oxime 11 (470 mg, 0.87 mmol), Na₂PdCl₄ (307 mg, 1.04 mmol), and NaOAc (75 mg, 0.914 mmol) in absolute EtOH (10 mL) and AcOH (10 mL) was stirred at rt for 3 days. The suspension was filtered and the solids were dissolved in CH₂Cl₂ (10 mL) and concentrated under reduced pressure to afford 405 mg (68% yield) of the dimeric organopalladium complex 13 as a light brown solid, which was taken to the next step without further purification. This reaction was conducted at larger scales up to 16 g of the starting oxime.

### Example 5

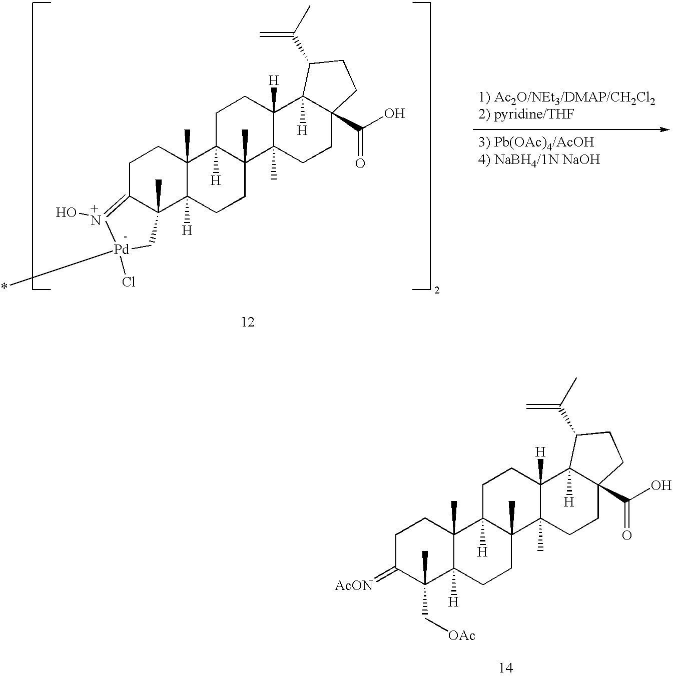

To a stirred solution of the dimeric organopalladium complex 12 (1.8 g, 1.47 mmol) in anhydrous CH₂Cl₂ (75 mL) at rt under N₂ was added Net₃ (1.2 mL, 8.82 mmol) and DMAP (27 mg, 0.22 mmol), followed by the addition of Ac₂O (0.57 mL, 6.04 mmol). After stirring at rt for 1 h, the resulting suspension was partitioned between H₂O (100 mL) and AcOH (10 mL); the organic layer was separated, dried (Na₂SO₄) and concentrated under reduced pressure to a yellow solid, which was dissolved in anhydrous THF (75 mL) and stirred at rt under N₂ as yridine (0.5 mL, 6.17 mmol) was added dropwise. After 15 min, the solution was cooled to −78° C. and stirring was continued under N₂ as a solution of Pb(Oac)₄ (1.33 g, 3.01 mmol) in AcOH (30 mL) was added dropwise. The reaction mixture was warmed up to rt and stirred for 16 h whereupon a solution of NaBH₄ (150 mg, 3.97 mmol) in aq. NaOH (1 N, 30 mL) was added dropwise. After 30 min, the black suspension was filtered and the filtrate was partitioned between H₂O (100 mL) and ether (100 mL). The organic layer was separated, dried (Na₂SO₄) and concentrated under reduced pressure to a light yellow oil which was purified by gradient silica gel flash column chromatography eluting with 10-50% EtOAc in hexanes to provide 700 mg (36% yield) of 14 as white solid: m.p. 186-189° C. (decomposed) ¹H NMR (500 MHz, CDCl₃) δ 0.82-1.8 (m), 0.88 (s), 0.96 (s), 0.97 (s), 1.15 (s), 1.69 (s), 1.98 (m), 2.05 (s), 2.16 (s), 2.25 (m), 2.60 (m), 2.65 (m), 3.0 (m), 4.13 (AB quartet, J=11.0 Hz), 4.61 (s), 4.74 (s).

### Example 6

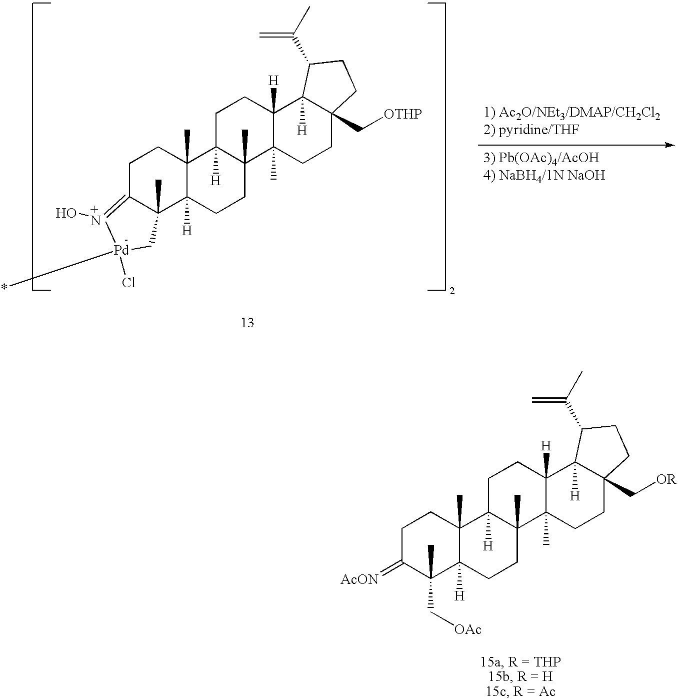

To a stirred solution of 13 (4.4 g, 3.23 mmol) in anhydrous CH₂Cl₂ (170 mL) at rt under N₂ was added Net₃ (2.2 mL, 15.78 mmol) and DMAP (55 mg, 0.45 mmol), followed by the addition of Ac₂O (1.1 mL, 11.63 mmol). After stirring at rt 1 h, the reaction was washed with H₂O (2×200 mL) and the organic layer was separated, dried (Na₂SO₄) and concentrated under reduced pressure to a yellow solid. This material was dissolved in anhydrous THF (170 mL) and stirred at rt under N₂ and pyridine (1.1 mL, 13.6 mmol) was added dropwise. After 15 min, the solution was cooled to 0° C. and stirring was continued under N₂ as a solution of Pb (Oac)₄ (2.94 mg, 6.63 mmol) in AcOH (60 mL) was added dropwise. After the addition was completed, the ice bath was removed and the suspension was stirred at rt for 16 h whereupon a solution of NaBH₄ (330 mg, 8.72 mmol) in NaOH (1 M, 60 mL) was added dropwise. After 15 min, the black suspension was filtered and the solids were washed with ether (30 mL). The combined filtrates were partitioned between H₂O (200 mL) and ether (200 mL). The organic layer was separated, washed with saturated NaHCO₃ (3×200 mL), dried (Na₂SO₄) and concentrated under reduced pressure to a light yellow oil which was purified by gradient silica gel flash column chromatography eluting with 10-30% EtOAc in hexanes to afford 1.1 g of a sample which was identified as a mixture of 15a and 15c, 651 mg of pure 15c (17% yield) and 1.01 g of 15b (28% yield) as white solids. Compound 15a. ¹H NMR (500 MHz, CDCl₃) δ 0.82-2.03 (m), 0.88 (s), 0.97 (s), 1.04 (s), 1.06 (s), 1.16 (s), 1.68 (s), 2.05 (s), 2.16 (s), 2.43 (m), 2.58 (m), 2.65 (m), 2.98 (d, J=9.5 Hz), 3.36 (d, J=9.5 Hz), 3.51 (d, J=9.5 Hz), 3.52 (m), 3.84 (m), 3.91 (d, J=8.5 Hz), 4.09 (d, J=11 Hz), 4.17 (d, J=11 Hz), 4.57 (m), 4.67 (s). Compound 15b: m.p. 117-120° C.; ¹H NMR (500 MHz, CDCl₃) δ 0.86-2.2 (m), 0.88 (s), 0.98 (s), 1.05 (s), 1.16 (s), 1.68 (s), 2.05 (s), 2.16 (s), 2.38 (m), 2.60 (m), 2.66 (m), 3.34 (d, J=10.5 Hz), 3.79 (d, J=11 Hz), 4.13 (AB quartet, J=11.0 Hz), 4.58 (s), 4.68 (m). Compound 15c: m.p. 107-109° C.; ¹H NMR (500 MHz, CDCl₃) δ 0.86-2.2 (m), 0.88 (s), 0.97 (s), 1.06 (s), 1.16 (s), 1.68 (s), 2.05 (s), 2.07 (s), 2.16 (s), 2.45 (m), 2.59 (m), 2.68 (s), 3.84 (d, J=11 Hz), 4.13 (AB quartet, J=11.0 Hz), 4.24 (d, J=11 Hz), 4.59 (s), 4.69 (s).

### Example 7

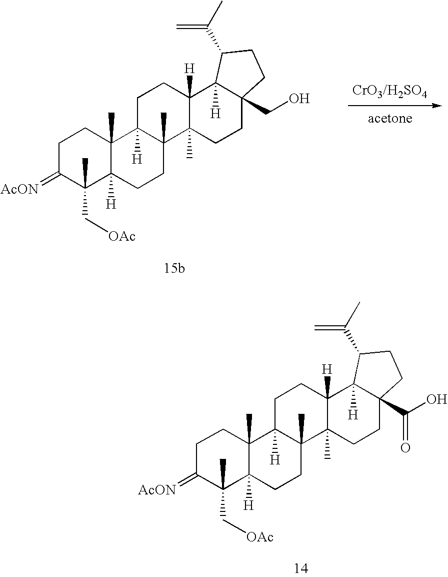

To a stirred solution of 15b (339 mg, 0.61 mmol) in acetone (5 mL) at 0° C. under N₂ was added the freshly prepared Jones' reagent (1.2 mL, 1.96 M, 2.35 mmol). As stirring was continued, a purple-brown precipitate was formed. After stirring at 0° C. for 1 h, aqueous Na₂S₂O₅ (500 mg in 5 mL of distilled water) was added and the dark green mixture was partitioned between EtOAc (30 mL) and water (30 mL). The aqueous layer was extracted with EtOAc (30 mL) and the combined organic layers were dried (Na₂SO₄) and concentrated under reduced pressure to a clear oil which was purified by gradient silica gel column chromatography eluting with 30-50% EtOAc in hexanes to give 201 mg (58% yield) of 14.

### Example 8

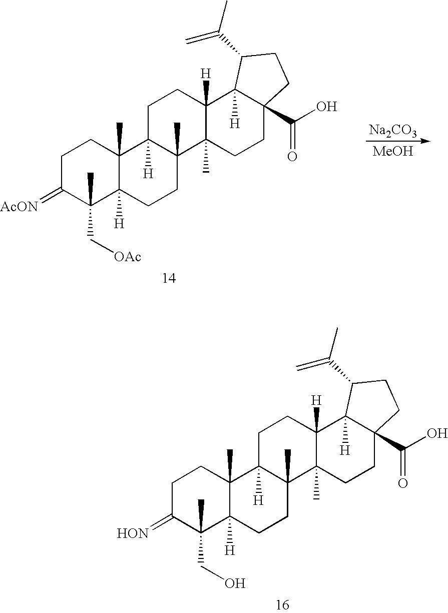

A suspension of 14 (0.6 g, 0.87 mmol) and Na₂CO₃ (0.5 g, 4.72 mmol) in MeOH (50 mL) was stirred at rt for 16 h, whereupon it was concentrated under reduced pressure and partitioned between EtOAc (50 mL) and H₂O (50 mL). The organic layer was washed with 1 N HCl (50 mL) and brine (50 mL), dried (Na₂SO₄) and concentrated under reduced pressure to afford 400 mg of white powder, which was purified by silica gel flash column chromatography elutin with 30-50% EtOAc in hexanes and 10% MeOH in CH₂Cl₂ to provide 140 mg (35% yield) of 16: m.p. 248-250° C. (decomposed); ¹H NMR (500 MHz, DMSO, d₆) δ 0.7-1.7 (m), 0.78 (s), 0.87 (s), 0.90 (s), 0.94 (s), 1.65 (s), 1.80 (m), 2.12 (m), 2.26 (m), 2.37 (m), 2.96 (m), 3.25 (m), 3.37 (m), 4.32 (t, J=5.5 Hz), 4.56 (s), 4.69 (s), 10.29 (s), 12.07 (bs).

### Example 9

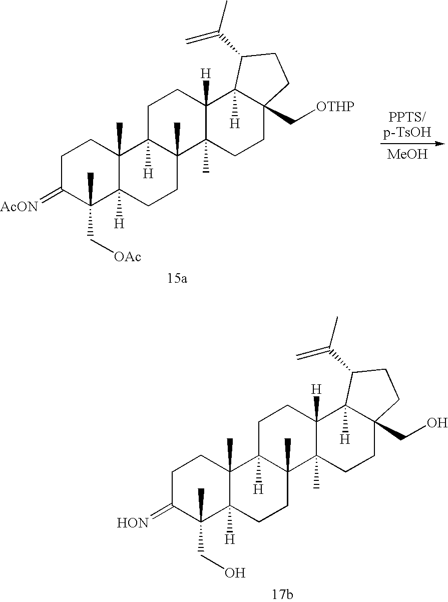

To a stirred solution of 15a (42 mg, 0.06 mmol) in MeOH (3 mL) at rt were added PPTS (20 mg, 0.08 mmol) and p-TsOH monohydrate (20 g, 0.10 mmol) all at once. After 16 h, the reaction mixture was diluted with EtOAc (10 mL) and washed with saturated NaHCO₃ (10 mL). The aqueous layer was extracted with EtOAc (10 mL) and the combined organic layers were dried (Na₂SO₄) and concentrated under reduced pressure to a crude residue which was purified by silica gel column chromatography eluting with gradient 30-50% EtOAc in hexanes to furnish 29 mg (95% yield) of 17b as white powder: m.p. 222-224° C.; ¹H NMR (500 MHz, CDCl₃) δ 0.8-2.1 (m), 0.97 (s), 1.01 (s), 1.06 (s), 1.68 (s), 2.10 (m), 2.38 (m), 2.91 (m), 3.06 (m), 3.33 (m), 3.48 (m), 3.61 (m), 3.79 (m), 4.58 (m), 4.68 (m), 6.67 (bs).

### Example 10

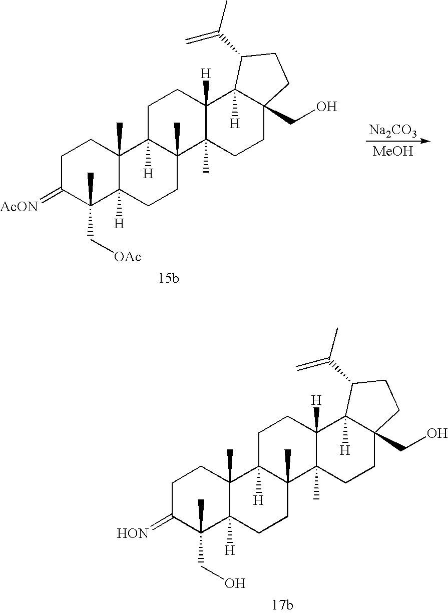

The same procedures shown above in Example 8 were employed to prepare compound 17b from 15b (300 mg, 0.54 mmol) and Na₂CO₃ (286 mg, 2.69 mmol) to afford 260 mg (100% yield) of the product as a white powder.

### Example 11

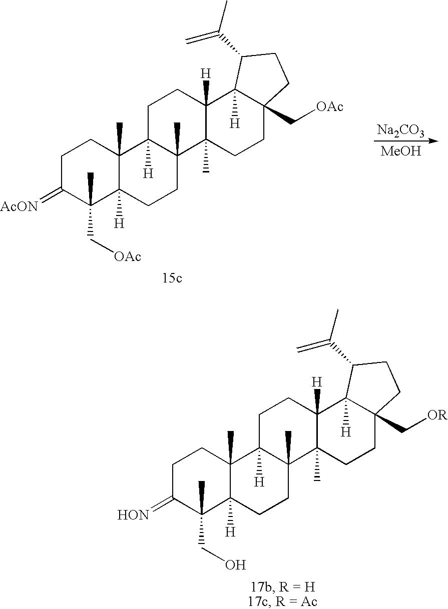

The same procedures shown above in Example 8 were employed to prepare compound 17c from 15c (596 mg, 1.0 mmol) and Na₂CO₃ (530 mg, 5.0 mmol). After purification by gradient silica gel flash column chromatography eluting with 30-50% EtOAc in hexanes, 98 mg (19% yield) of 17c and 328 mg (69% yield) of 17b were obtained. ¹H NMR (500 MHz, CDCl₃) spectrum of 17b was identical to that of an authentic sample prepared from a procedure described above. Compound 17c: ¹H NMR (500 MHz, CD₃OD) δ 0.9-2.2 (m), 0.96 (s), 0.97 (s), 1.00 (s), 1.07 (s), 1.67 (s), 2.44 (m), 2.97 (bs), 3.08 (m), 3.54 (AB quartet), 3.85 (d, J=11 Hz), 4.24 (d, J=11 Hz), 4.58 (m), 4.68 (m), 6.89 (bs).

### Example 12

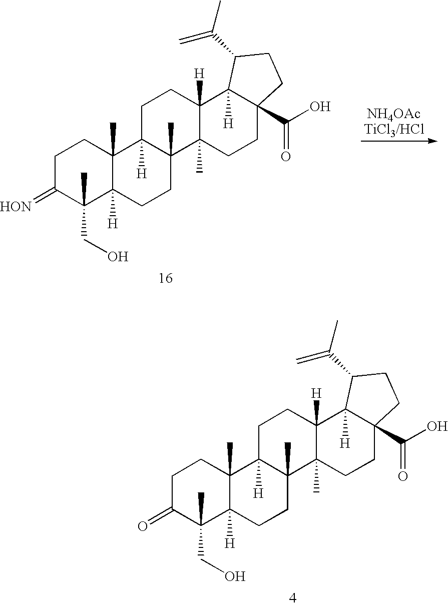

To a stirred solution of NH₄Oac (952 mg, 12.35 mmol) in water (15 mL) at rt was added dropwise a solution of 10% TiCl₃ in aqueous 20-30% HCl (4 mL, 3.09 mmol), followed by dropwise addition of 16 (200 mg, 0.41 mmol) in THF (15 mL) over 10 min. After 16 h, the resulting dark mixture was extracted with ether (3×30 mL). The combined organic layers were dried (Na₂SO₄) and concentrated under reduced pressure to an oily residue which was purified by gradient silica gel flash column chromatography eluting with 10-40% EtOAc in hexanes to afford 54 mg (28% yield) of 23-hydroxybetulonic acid 4 as a white powder: m.p. 218-220° C.; ¹H NMR (500 MHz, CDCl₃) δ 0.9-1.8 (m), 0.98 (s), 0.99 (s), 1.00 (s), 1.04 (s), 1.69 (s), 1.98 (m), 2.27 (m), 2.62 (m), 3.00 (m), 3.52 (AB quartet), 4.61 (m), 4.74 (m).

### Example 13

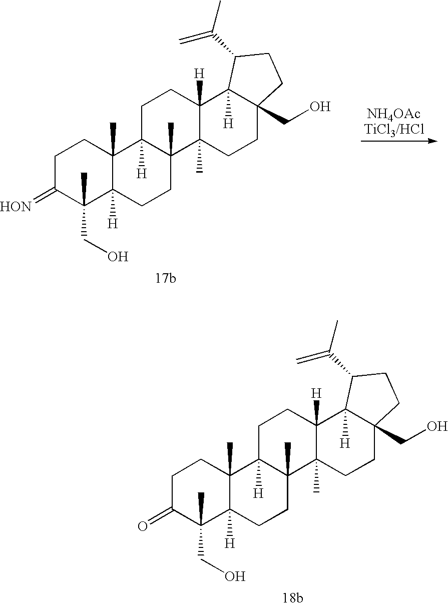

The same procedures shown above in Example 12 were employed to prepare compound 18b from 17b (100 mg, 0.21 mmol), NH₄Oac (440 mg, 5.71 mmol) and 10% TiCl₃ in aqueous 20-30% HCl (3 mL, 2.34 mmol). After purification by gradient silica gel flash column chromatography eluting with 30-50% EtOAc in hexanes, 75 mg (77% yield) of 18b was obtained as a white powder: m.p. 233-235° C.; ¹H NMR (500 MHz, CDCl₃) δ 0.96-1.76 (m), 0.99 (s), 1.00 (s), 1.04 (s), 1.08 (s), 1.68 (s), 1.85 (m), 1.96 (m), 2.27 (m), 2.33 (t, J=6.5 Hz), 2.39 (m), 2.61 (ddd, J=7, 13, 16.5 Hz), 3.34 (d, J=11 Hz), 3.41 (dd, J=6.5, 11 Hz), 3.64 (dd, J=7, 11.5 Hz), 3.79 (d, J=10.5 Hz), 4.58 (m), 4.68 (m).

### Example 14

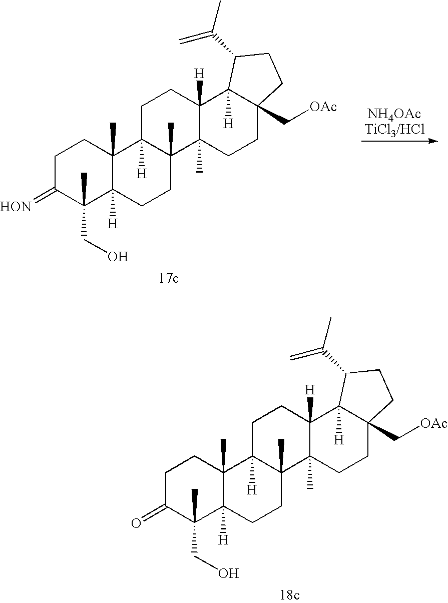

The same procedures shown above in Example 12 were employed to prepare compound 18c from 17c (81 mg, 0.15 mmol), NH₄Oac (500 mg, 6.48 mmol) and 10% TiCl₃ in aqueous 20-30% HCl (2 mL, 1.54 mmol). After purification by gradient silica gel flash column chromatography eluting with 10-30% EtOAc in hexanes, 48 mg (62% yield) of 18c was obtained as a white powder. ¹H NMR (500 MHz, CDCl₃) δ 0.8-2.0 (m), 0.98 (s), 1.00 (s), 1.04 (s), 1.09 (s), 1.68 (s), 2.07 (s), 2.27 (m), 2.32 (t, J=7 Hz), 2.45 (m), 2.62 (ddd, J=7, 13, 16.5 Hz), 3.40 (dd, J=6.5, 11 Hz), 3.64 (dd, J=6.5, 11.5 Hz), 3.85 (d, J=10.5 Hz), 4.26 (d, J=11 Hz), 4.59 (m), 4.69 (d, J=2 Hz).

### Example 15

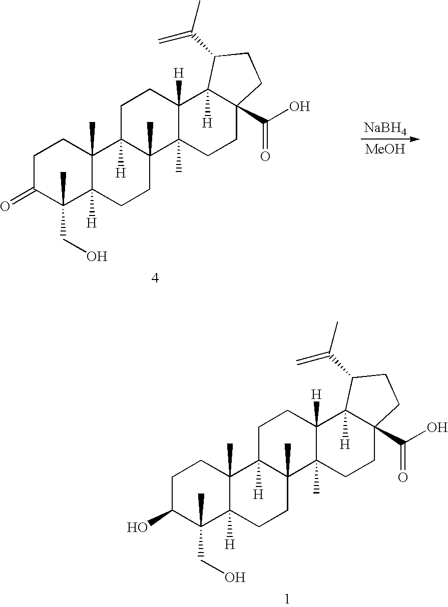

To a stirred solution of 23-hydroxybetulonic acid 4 (40 mg, 0.09 mmol) in MeOH (3 mL) at rt under N₂ was added NaBH₄ (16 mg, 0.42 mmol) all at once. After 16 h, the reaction mixture was diluted with EtOAC (20 mL) and acidified with 1 N HCl (20 mL). The aqueous layer was extracted with EtOAC (20 mL) and the combined organic layers were dried (Na₂SO₄) and concentrated under reduced pressure to a solid residue which was purified by gradient silica gel column chromatography eluting with 30-50% EtOAc in hexanes and then 10% MeOH in CH₂Cl₂ to afford 34 mg (85% yield) of 23-hydroxybetulinic acid 1 as white solid: m.p. 258-259° C.; ¹H NMR (500 MHz, CD₃OD) δ 0.8-1.8 (m), 0.86 (s), 0.87 (s), 0.93 (s), 0.97 (s), 1.68 (s), 1.97 (m), 2.21 (m), 2.98 (m), 3.56 (AB quartet), 3.62 (m), 4.60 (m), 4.73 (m).

### Example 16

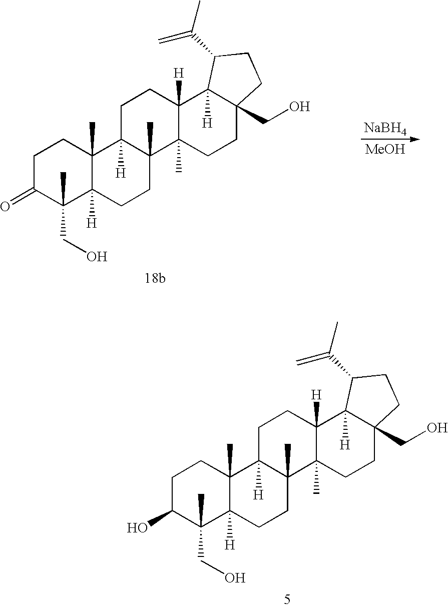

To a stirred solution of 18b (52 mg, 0.11 mmol) in MeOH (3 mL) at rt under N₂ was added NaBH₄ (39 mg, 0.22 mmol) all at once. After 16 h, the reaction mixture was diluted with EtOAC (20 mL) and washed with saturated NaHCO₃ (10 mL). The aqueous layer was extracted with EtOAC (20 mL) and the combined organic layers were dried (Na₂SO₄) and concentrated under reduced pressure to a solid which was purified by gradient silica gel column chromatography eluting with 30-50% EtOAc in hexanes to deliver 46 mg (89% yield) of 5 as white solid: m.p. 256-260° C.; ¹H NMR (500 MHz, CD₃OD) δ 0.8-2.02 (m), 0.87 (s), 0.97 (s), 1.02 (s), 1.68 (s), 2.22 (bs), 2.39 (m), 2.46 (bs), 3.33 (d, J=9.5 Hz), 3.42 (d, J=10.5 Hz), 3.62 (t, J=8 Hz), 3.72 (d, J=10 Hz), 3.79 (d, J=9.5 Hz), 4.58 (m), 4.68 (m).

### Example 17

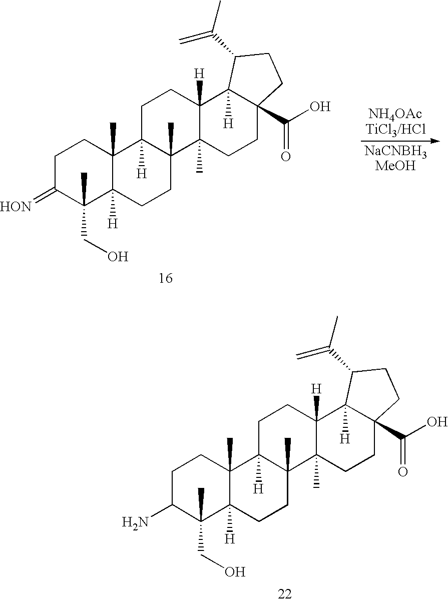

To a stirred solution of 16 (170 mg, 0.35 mmol) and NH₄Oac (600 mg, 7.71 mmol) in MeOH (40 mL) at rt under N₂ was added NaCNBH₃ (600 mg, 9.55 mmol) all at once. The reaction mixture was cooled to 0° C. onto which a solution of 10% TiCl₃ in aqueous 20-30% HCl (2 mL, 1.62 mmol) was added dropwise over a period of 10 min. [Note: The addition of NaCNBH₃ is accompanied by rapid evolution of H₂. A slow addition of the hydride reagent in a well vented reaction flask at 0° C. under N₂ is highly recommended]. The ice bath was removed and stirring continued at rt for 3 days. The heterogeneous mixture was concentrated to ca. 10 mL in volume under reduced pressure, diluted with EtOAc (50 mL) and washed with 10% NH₄OH (50 mL). The solids were filtered off and discarded. The organic layer was separated and the aqueous layer acidified to pH of 2 and extracted with EtOAc (3×50 mL). All the organic layers were combined, dried (Na₂SO₄) and concentrated under reduced pressure to an orange solid which was purified by gradient silica gel flash column chromatography eluting with 10:90:1-20:80:1 of MeOH:CH₂Cl₂:Conc. NH₄OH to afford 20 mg (12% yield) of 22 as a white powder: m.p. >299° C.; ¹H NMR (500 MHz, CD₃OD) δ 0.6-1.9 (m), 0.68 (s), 0.78 (s), 0.87 (s), 0.93 (s), 1.64 (s), 2.11 (m), 2.23 (m); 2.95 (m), 3.02 (m), 3.33 (m), 4.56 (s), 4.68 (s); Positive ESI-MS, m/e 472.3 (M+H⁺).

### Example 18

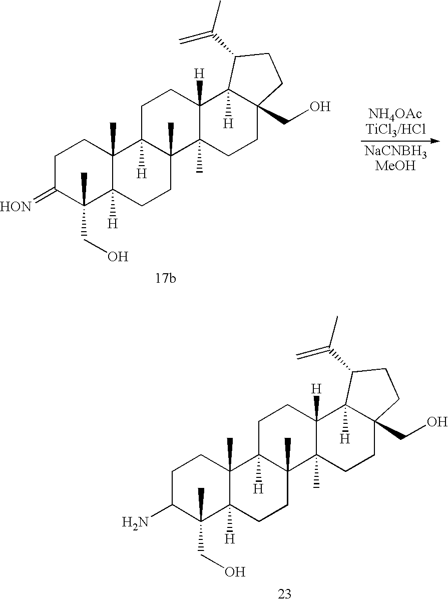

The same procedures shown above in Example 17 were employed to prepare compound 23 from 17b (255 mg, 0.54 mmol), NH₄Oac (601 mg, 7.73 mmol), NaCNBH₃ (643 mg, 10.2 mmol), and 10% TiCl₃ in aqueous 20-30% HCl (2 mL, 1.62 mmol) was added dropwise over a period of 10 min. The ice bath was removed and stirring was continued at rt for 16 h. After purification by gradient silica gel flash column chromatography eluting with 10:90:1 of MeOH:CH₂Cl₂:Conc. NH₄OH, 64 mg (26% yield) of 23 was obtained as a white powder: m.p. 172-174° C.; ¹H NMR (500 MHz, CD₃OD) δ 0.8-2.02 (m), 0.72 (s), 0.88 (s), 1.01 (s), 1.07 (s), 1.68 (s), 2.410 (m), 2.73 (dd, J=7 and 9 Hz), 3.28 (d, J=11 Hz), 3.34 (d, J=11 Hz), 3.47 (d, J=11 Hz), 3.73 (dd, J=1.5, 11 Hz), 4.56 (m), 4.68 (d, J=2.5 Hz); Positive ESI-MS, m/e 458.3 (M+H⁺).

### Example 19

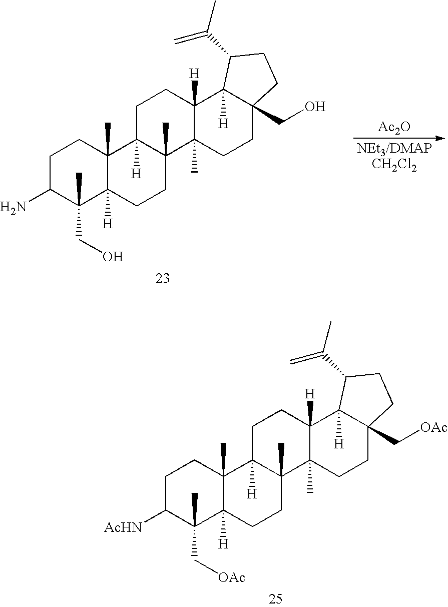

To a stirred solution of 23 (51 mg, 0.11 mmol) in anhydrous CH₂Cl₂ (5 mL) ar rt under N₂ were added Net₃ (0.15 mL, 1.11 mmol) and N,N-DMAP (4 mg, 0.03 mmol), followed by the addition of Ac₂O (0.05 mL, 0.55 mmol). After stirring at rt 2 h, the resulting mixture was diluted with EtOAc (30 mL) and quenched with 1N HCl (30 mL). The organic layer was separated, washed with saturated NaHCO₃ (30 mL), dried (Na₂SO₄) and concentrated under reduced pressure to a crude material which was purified by silica gel flash column chromatography eluting with EtOAc and 10% MeOH in CH₂Cl₂ to furnish 70 mg (quantitative yield) of 25 as a white powder: m.p. 127-130° C.; ¹H NMR (500 MHz, CDCl₃) δ 0.7-2.1 (m), 0.71 (s), 0.84 (s), 0.97 (s), 1.03 (s), 1.68 (s), 1.95 (s), 2.06 (s), 2.09 (s), 2.44 (m), 3.59 (d, J=12 Hz), 3.83 (m), 4.03 (m), 4.24 (d, J=11 Hz), 4.59 (m), 4.69 (d, J=1.5 Hz), 5.12 (d, J=10.5 Hz).

### Example 20

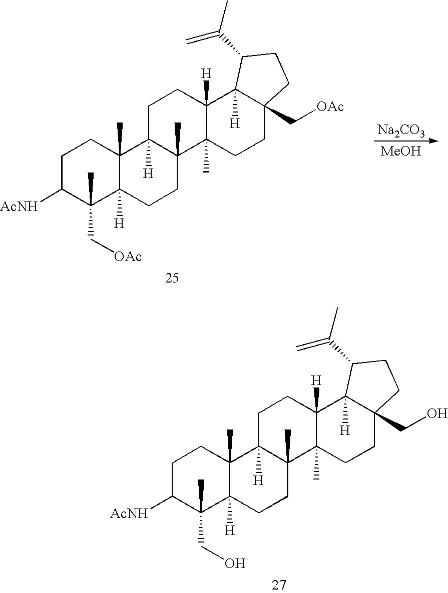

A suspension of 25 (52 mg, 0.09 mmol) and Na₂CO₃ (50 mg, 0.47 mmol) in MeOH (5 mL) was stirred at rt for 16 h. The suspension was concentrated under reduced pressure and partitioned between EtOAc (30 mL) and H₂O (30 mL). The organic layer was separated, washed with 1 N HCl (30 mL) and saturated NaHCO₃ (300 mL), dried (Na₂SO₄) and concentrated under reduced pressure to a crude product which was purified by silica gel flash column chromatography eluting with 10:90:1-20:80:1 of MeOH:CH₂Cl₂:Conc. NH₄OH to afford 36 mg (81% yield) of 27 as a white powder: m.p. 146-149° C.; ¹H NMR (500 MHz, CDCl₃) δ 0.8-2.1 (m), 0.55 (s), 0.84 (s), 0.99 (s), 1.01 (s), 1.68 (s), 2.01 (s), 2.39 (m), 2.86 (d, J=13 Hz), 3.33 (d, J=11 Hz), 3.38 (d, J=12.5 Hz), 3.79 (d, J=11 Hz), 3.89 (m), 4.08 (bs), 4.57 (m), 4.68 (d, J=2.5 Hz), 5.30 (d, J=9.5 Hz).

### Example 21

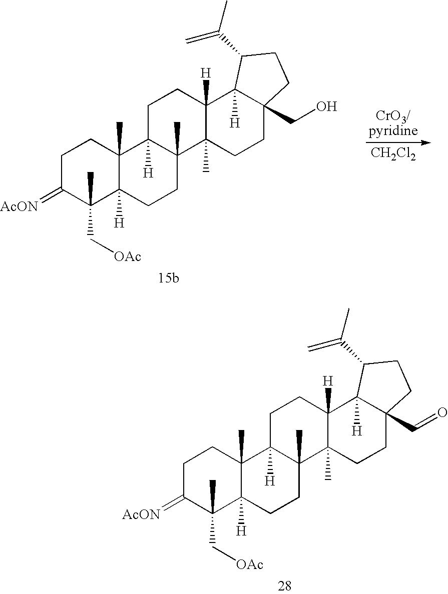

To a stirred solution of pyridine (0.9 mL, 10.8 mmol) in anhydrous CH₂Cl₂ (20 mL) at rt under N₂ was added CrO₃ (540 mg, 5.39 mmol) all at once. The resulting dark brown suspension was stirred at rt for 1 h and then cooled to 0° C. into which a solution of 15b (500 mg, 0.89 mmol) in anhydrous CH₂Cl₂ (10 mL) was added dropwise. The suspension was stirred at 0° C. for additional hour and passed through a short path of silica gel (350 mL) eluting with CH₂Cl₂ (100 mL) and EtOAc (400 mL) to give 487 mg (98% yield) of the aldehyde 28 as a white solid: m.p. 146-149° C.; ¹H NMR (500 MHz, CD₃OD) δ 0.9-2.2 (m), 0.91 (s), 0.97 (s), 1.02 (s), 1.15 (s), 1.71 (s), 2.01 (s), 2.13 (s), 2.68 (m), 2.90 (m), 4.11 (d, J=10.5 Hz), 4.19 (d, J=11 Hz), 4.62 (m), 4.75 (m), 4.83 (s), 9.65 (d, J=1.5H).

### Example 22

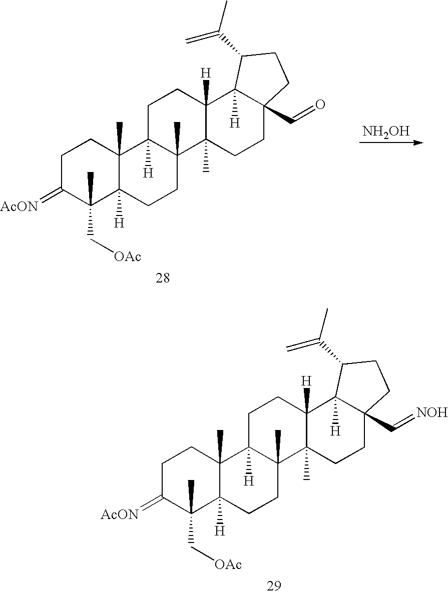

To a stirred solution of 28 (1.07 g, 1.93 mmol) and pyridine (0.63 mL, 7.73 mmol) in absolute EtOH (30 mL) at rt under N₂ was added NH₂OH hydrochloride (188 mg, 2.70 mmol) all at once. After stirring at rt for 16 h, the reaction mixture was diluted with EtOAc (100 mL) and quenched with H₂O (100 mL). The organic layer was separated, dried (Na₂SO₄) and concentrated under reduced pressure to afford 980 mg (89% yield) of 29 as a white solid: m.p. 128-132° C.; ¹H NMR (500 MHz, CD₃OD) δ 0.9-2.0 (m), 0.92 (s), 1.02 (s), 1.05 (s), 1.15 (s), 1.70 (s), 2.01 (s), 2.13 (s), 2.57 (m), 2.65 (m), 2.72 (m), 4.15 (AB quartet, J=11 Hz), 4.60 (m), 4.72 (m), 7.47 (s).

### Example 23

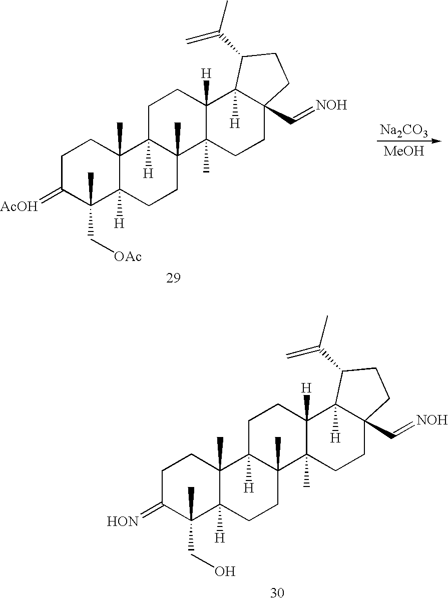

The same procedures shown above in Example 8 were employed to prepare compound 30 from 29 (308 mg, 0.54 mmol) and Na₂CO₃ (300 mg, 2.83 mmol) to produce 238 mg (91% yield) of 30 as a white solid: m.p. 194-199° C.; ¹H NMR (500 MHz, CD₃OD) δ 0.9-2.0 (m), 0.95 (s), 0.97 (s), 1.02 (s), 1.06 (s), 1.70 (s), 2.24 (m), 2.56 (m), 2.90 (m), 3.52 (AB quartet, J=11.5 Hz), 4.59 (s), 4.71 (s), 7.48 (s).

### Example 24

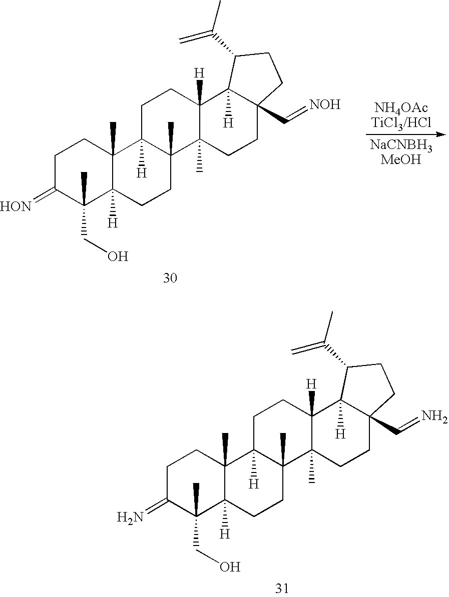

The same procedures shown above in Example 17 were employed to prepare compound 31 from 30 (220 mg, 0.45 mmol), NH₄Oac (1.0 g, 12.85 mmol), NaCNBH₃ (1.0 g, 16.0 mmol), and 10% TiCl₃ in aqueous 20-30% HCl (4 mL, 3.10 mmol). After purification by gradient silica gel flash column chromatography eluting with 10-20% MeOH in CH₂Cl₂ 72 mg (35% yield) of 31 was obtained as a white solid: m.p. >299° C.; ¹H NMR (500 MHz, CD₃OD) δ 0.86-2.08 (m), 0.94 (s), 0.96 (s), 1.04 (s), 1.11 (s), 1.71 (s), 2.46 (m), 2.74 (d, J=13 Hz), 3.13 (d, J=12 Hz), 3.18 (dd, J=4.5 and 12.5 Hz), 3.42 (d, J=11 Hz), 3.66 (d, J=11 Hz), 4.62 (m), 4.74 (m); positive ESI-MS, m/e 457.3 (M+H⁺).

### Example 25

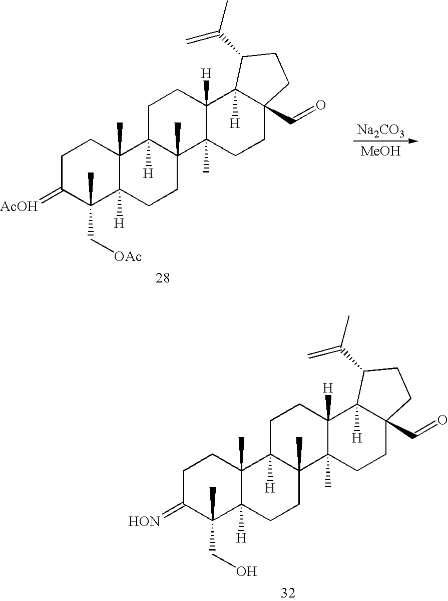

The same procedures shown above in Example 8 were employed to prepare compound 32 from 28 (100 mg, 0.18 mmol) and Na₂CO₃ (96 mg, 0.90 mmol) to afford 90 mg (100% yield) of 32 as a white solid: m.p. 210-215° C.; ¹H NMR (500 MHz, CD₃OD) δ 0.9-2.1 (m), 0.94 (s), 0.96 (s), 0.98 (s), 1.02 (s), 1.71 (s), 2.24 (m), 2.90 (m), 3.41 (d, J=11 Hz), 3.61 (d, J=11 Hz), 4.62 (m), 4.75 (m), 9.66 (d, J=1.5 Hz).

### Example 26

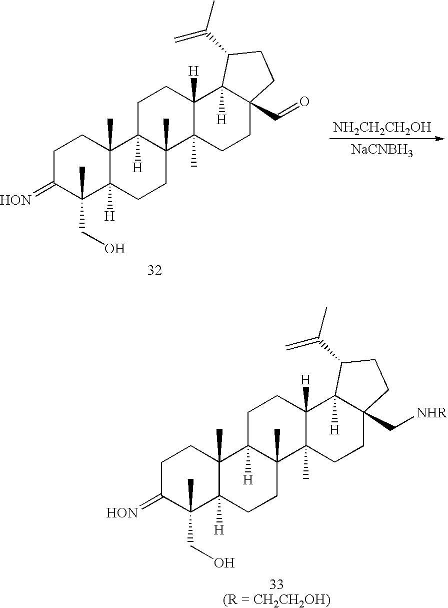

To a stirred solution of 32 (264 mg, 0.56 mmol) in anhydrous MeOH (20 mL) at rt under N₂ were added ethanolamine (0.3 mL, 4.97 mmol), NaCNBH₃ (300 mg, 4.77 mmol) and AcOH (5 drops) in succession. Stirring was continued at rt for 3 days whereupon the reaction mixture was diluted with EtOAc (100 mL) and basified with 10% NaOH (50 mL). The organic layer was separated, dried (Na₂SO₄) and concentrated under reduced pressure to a white solid which was purified by silica gel column chromatography eluting with 30% EtOAc in hexanes and 10-20% MeOH in CH₂Cl₂ to provide 218 mg (75% yield) of 33 (R=CH₂CH₂OH) as a white solid: m.p. 214-218° C.; ¹H NMR (500 MHz, CD₃OD) δ 0.90-1.98 (m), 0.96 (s), 0.97 (s), 1.04 (s), 1.14 (s), 1.71 (s), 2.04 (m), 2.25 (m), 2.48 (m), 2.72 (d, J=12.5 Hz), 2.91 (m), 3.10 (m), 3.20 (d, J=12.5 Hz), 3.27 (t, J=6 Hz), 3.42 (d, J=11 Hz), 3.58 (t, J=5.5 Hz), 3.62 (d, J=11 Hz), 3.82 (m), 4.62 (m), 4.73 (m). Positive ESI-MS, m/e 515.3 (M+H⁺).

### Example 27

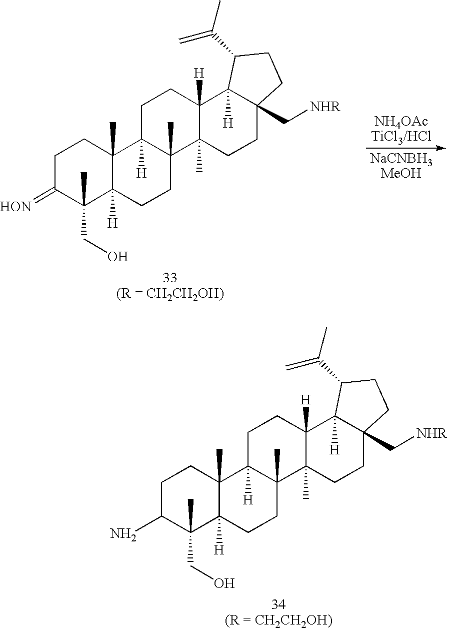

The same procedures shown above in Example 17 were employed to prepare compound 34 from 33 (R=CH₂CH₂OH) (200 mg, 0.38 mmol), NH₄Oac (906 mg, 11.64 mmol), NaCNBH₃ (878 mg, 13.97 mmol), and 10% TiCl₃ in aqueous 20-30% HCl (4 mL, 3.10 mmol). After purification by gradient silica gel flash column chromatography eluting with 10-20% MeOH in CH₂Cl₂, 180 mg (93% yield) of 34 (R=CH₂CH₂OH) was obtained as a white solid: m.p. >299° C.; ¹H NMR (500 MHz, CD₃OD) δ 0.86-2.0 (m), 0.93 (s), 0.96 (s), 1.04 (s), 1.12 (s), 1.71 (s), 2.08 (m), 2.50 (m), 2.88 (d, J=13 Hz), 3.21 (m), 3.42 (d, J=11 Hz), 3.66 (d, J=10.5 Hz), 3.88 (t, J=5 Hz), 4.62 (m), 4.74 (m); positive ESI-MS, m/e 501.3 (M+H⁺).

### Example 28

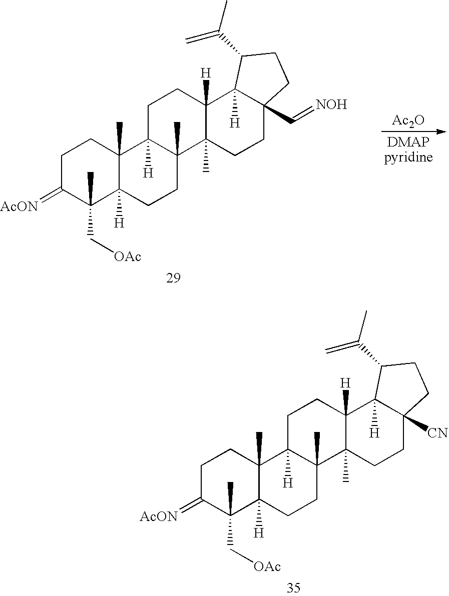

To a stirred solution of 29 (694 mg, 1.22 mmol) and DMAP (298 mg, 2.44 mmol) in pyridine (10 mL) at rt under N₂ was added dropwise Ac₂O (1.15 mL, 12.2 mmol). The reaction mixture was then heated at 90° C. for 16 h, allowed to cool down to rt slowly and diluted with EtOAc (100 mL). The homogeneous mixture was acidified with 0.5 N HCl (50 mL) and the organic layer was separated, washed with saturated NaHCO₃ (50 mL), dried (Na₂SO₄) and concentrated under reduced pressure to a crude material which was purified by silica gel flash column chromatography eluting with 10-20% EtOAc in hexanes to afford 460 mg (74% yield) of 35 as a white solid: m.p. 108-111° C.; ¹H NMR (500 MHz, CDCl₃) δ 0.90-2.24 (m), 0.90 (s), 0.95 (s), 1.11 (s), 1.17 (s), 1.68 (s), 2.05 (s), 2.17 (s), 2.67 (m), 4.14 (AB quartet, J=11H), 4.66 (m), 4.77 (m); positive ESI-MS, m/e 551.2 (M+H⁺).

### Example 29

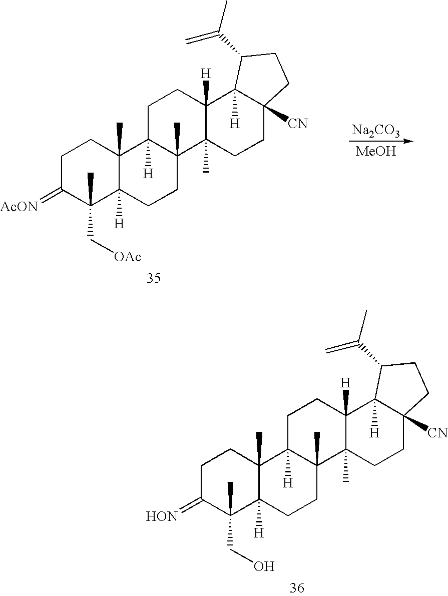

The same procedures shown above in Example 8 were employed to prepare compound 36 from 35 (113 mg, 0.19 mmol) and Na₂CO₃ (105 mg, 0.99 mmol) to afford 100 mg (100% yield) of 36 as a white solid: m.p. 232-236° C.; ¹H NMR (500 MHz, CD₃OD) δ 0.9-2.2 (m), 0.976 (s), 0.978 (s), 1.01 (s), 1.13 (s), 1.70 (s), 2.25 (m), 2.60 (m), 2.90 (m), 3.52 (AB quartet, J=11.5 Hz), 4.67 (m), 4.78 (m).

### Example 30

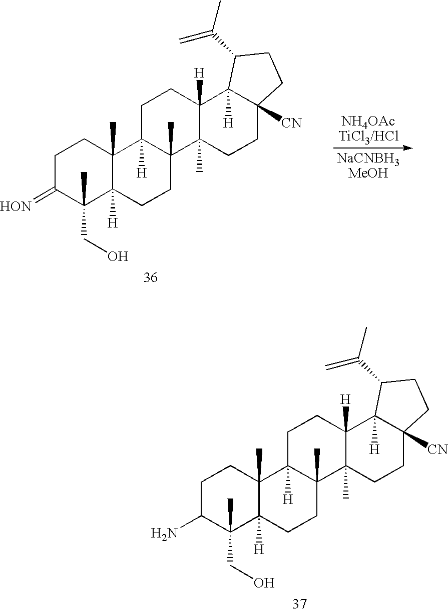

The same procedures shown above in Example 17 were employed to prepare compound 37 from 36 (88 mg, 0.18 mmol), NH₄Oac (0.5 g, 6.42 mmol), NaCNBH₃ (0.5 g, 7.95 mmol), and 10% TiCl₃ in aqueous 20-30% HCl (2 mL, 1.55 mmol). After purification by gradient silica gel flash column chromatography eluting with 10:90:1-20:80:1 of MeOH:CH₂Cl₂:Conc. NH₄OH, 50 mg (61% yield) of 37 was obtained as a white powder: m.p. >299° C.; ¹H NMR (500 MHz, CD₃OD) δ 0.9-2.2 (m), 0.95 (s), 0.96 (s), 1.00 (s), 1.11 (s), 1.70 (s), 2.61 (m), 3.18 (dd, J=4.5 and 12.5 Hz), 3.42 (d, J=10.5 Hz), 3.66 (d, J=10.5 Hz), 4.66 (m), 4.78 (m).

### Example 31

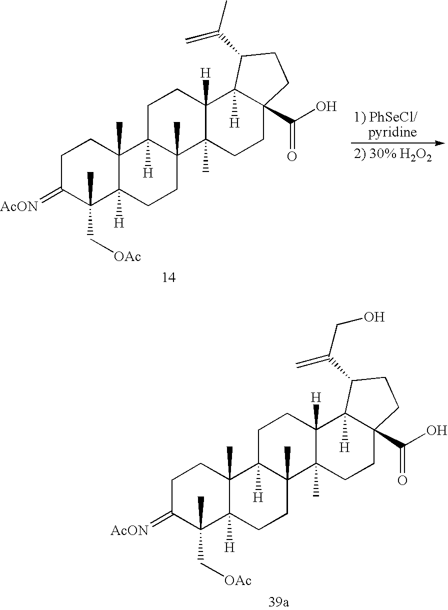

To a stirred solution of benzeneselenenyl chloride (561 mg, 2.93 mmol) in anhydrous CH₂Cl₂ (30 mL) at 0° C. under N₂ was added pyridine (1.42 mL, 17.58 mmol). The resulting light yellow solution was stirred for an additional 0.5 h at 0° C. into which a solution of 14 (1.33 g, 2.34 mmol) in anhydrous CH₂Cl₂ (10 mL) was added dropwise. After 1 h, a solution of 30% aqueous H₂O₂ (1.1 mL, 11.1 mmol) was added dropwise and stirring was continued at 0° C. for 2 h. The heterogeneous mixture was diluted with EtOAc (100 mL). The organic layer was separated, washed with 1 N HCl (100 mL), dried (Na₂SO₄) and concentrated under reduced pressure to a foamy residue which was purified by gradient silica gel column chromatography eluting with 30-50% EtOAc in hexanes and then 10% MeOH in CH₂Cl₂ to give 443 mg (32% yield) of 39a: m.p. 189-193° C.; ¹H NMR (500 MHz, CD₃OD) δ 0.86-2.38 (m), 0.88 (s), 0.96 (s), 0.98 (s), 1.15 (s), 2.06 (s), 2.17 (s), 2.47 (m), 2.62 (m), 2.89 (m), 4.04 (m), 4.92 (s), 4.98 (s).

### Example 32

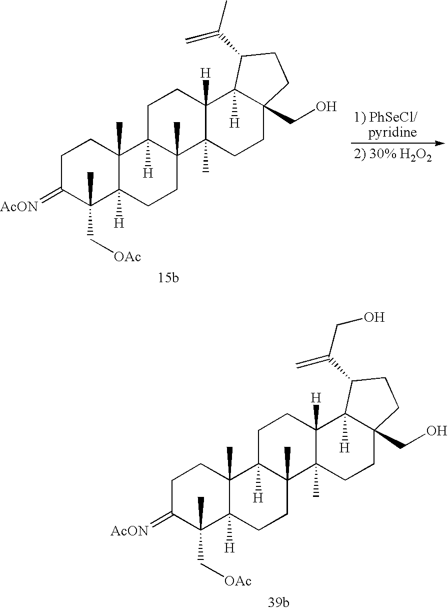

The same procedures shown above in Example 31 were employed to prepare compound 39b from 15b (600 mg, 1.00 mmol), benzeneselenenyl chloride (423 mg, 2.21 mmol), pyridine (0.48 mL, 6.02 mmol), and 30% aqueous H₂O₂ (1.1 mL, 11.1 mmol). After purification by gradient silica gel column chromatography eluting with 20-100% EtOAc in hexanes, 289 mg (47% yield) of 39b was obtained as a white solid: m.p. 150-155° C.; ¹H NMR (500 MHz, CD₃OD) δ 0.90-2.2 (m), 0.92 (s), 1.02 (s), 1.04 (s), 1.11 (s), 1.16 (s), 2.02 (s), 2.13 (s), 2.31 (m), 2.39 (m), 2.64 (m), 2.73 (m), 3.28 (d, J=12 Hz), 3.73 (d, J=11 Hz), 4.04 (m), 4.16 (m), 4.86 (s), 4.94 (m).

### Example 33

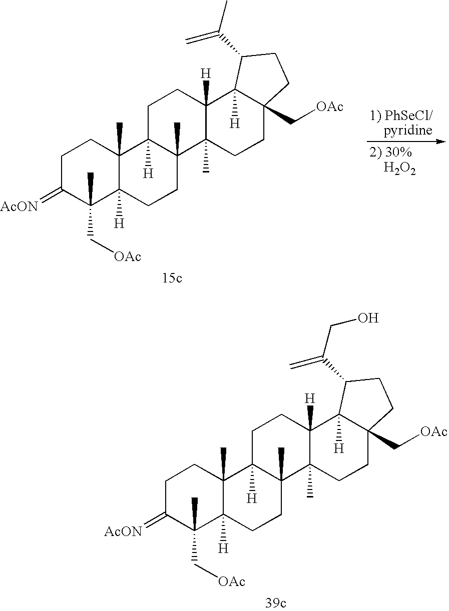

The same procedures shown above in Example 31 were employed to prepare compound 39c from 15c (1.2 g, 2.00 mmol), benzeneselenenyl chloride (846 mg, 4.42 mmol), pyridine (1.0 mL, 12.36 mmol), and 30% aqueous H₂O₂ (2.2 mL, 21.5 mmol). After purification by gradient silica gel column chromatography eluting with 20-100% EtOAc in hexanes, 653 mg (53% yield) of 39c was obtained as a white solid: m.p. 113-116° C.; ¹H NMR (500 MHz, CDCl₃) δ 0.8-2.2 (m), 0.88 (s), 0.98 (s), 1.06 (s), 1.16 (s), 2.05 (s), 2.07 (s), 2.16 (s), 2.34 (m), 2.58 (m), 2.68 (m), 3.85 (d, J=11.5 Hz), 4.11 (m), 4.23 (d, J=11 Hz), 4.90 (s), 4.96 (d, J=1 Hz).

### Example 34

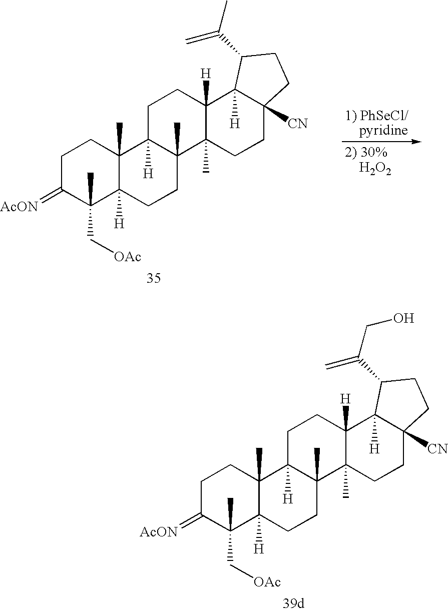

The same procedures shown above in Example 31 were employed to prepare compound 39d from 35 (370 mg, 0.65 mmol), benzeneselenenyl chloride (274 mg, 1.43 mmol), pyridine (0.32 mL, 3.90 mmol), and 30% aqueous H₂O₂ (0.68 mL, 6.5 mmol). After purification by gradient silica gel column chromatography eluting with 10-50% EtOAc in hexanes, 125 mg (41% yield) of 39d, along with 150 mg of unreacted 35 were obtained. Compound 39d: m.p. 141-143° C.; ¹H NMR (500 MHz, CDCl₃) δ 0.89-2.2 (m), 0.90 (s), 0.95 (s), 1.11 (s), 1.17 (s), 2.05 (s), 2.17 (s), 2.30 (m), 2.63 (m), 4.12 (m), 4.14 (AB quartet, J=11 Hz), 4.92 (m), 5.02 (m).

### Example 35

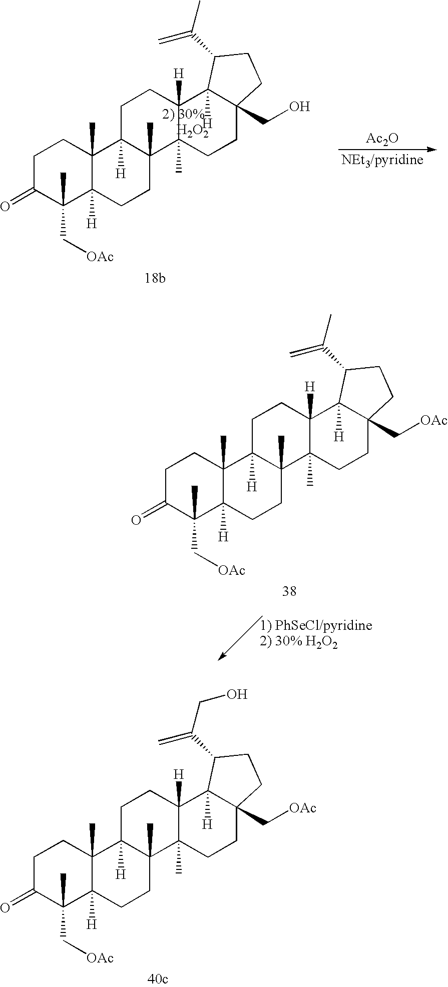

To a stirred solution of 18b (544 mg, 1.19 mmol), Net₃ (0.86 mL, 6.19 mmol) and DMAP (38 mg, 0.31 mmol) in anhydrous CH₂Cl₂ (20 mL) at rt under N₂ was added Ac₂O (0.35 mL, 3.7 mmol) dropwise. After 5 min, the resulting mixture was diluted with EtOAc (100 mL) and acidified with 1N HCl (100 mL). The organic layer was separated, washed with saturated NaHCO₃ (100 mL), dried (Na₂SO₄) and concentrated under reduced pressure to an oil which was purified by gradient silica gel flash column chromatography eluting with 10-40% EtOAc in hexanes to afford 600 mg (94% yield) of 38 as a white powder. ¹H NMR (500 MHz, CD₃OD) δ 0.9-2.1 (m), 0.94 (s), 0.99 (s), 1.00 (s), 1.08 (s), 1.68 (s), 2.03 (s), 2.07 (s), 2.46 (m), 3.86 (d, J=11 Hz), 4.04 (AB quartet), 4.25 (dd, J=1.5 and 11 Hz), 4.59 (m), 4.69 (m).

To a stirred solution of PhSeCl (425 mg, 2.22 mmol) in anhydrous CH₂Cl₂ (15 mL) at 0° C. under N₂ was added pyridine (0.54 mL, 6.66 mmol), and the dark orange color turned to light yellow. The reaction solution was stirred at 0° C. for another 0.5 h whereupon a solution of 38 (0.6 g, 1.11 mmol) in anhydrous CH₂Cl₂ (10 mL) was added. The reaction mixture was stirred for 4 h while the temperature was gradually increased from 0° C. to rt, into which another portion of PhSeCl (100 mg, 0.52 mmol) was added. After stirring at rt for an additional hour, the reaction mixture was cooled down to 0° C. and 30% aqueous H₂O₂ (1.13 mL, 11.10 mmol) was added dropwise. Stirring was continued for 1 h at 0° C. The heterogeneous mixture was diluted with EtOAc (100 mL). The organic layer was separated, washed with 1 N HCl (100 mL) and saturated NaHCO₃ (100 mL), dried (Na₂SO₄) and concentrated under reduced pressure to a foamy residue which was purified by gradient silica gel column chromatography eluting with 20-50% EtOAc in hexanes to afford 400 mg (65% yield) of 40c as a white solid: m.p. 117-119° C.; ¹H NMR (500 MHz, CDCl₃) δ 0.9-2.2 (m), 0.94 (s), 1.00 (s), 1.08 (s), 2.03 (s), 2.07 (s), 2.44 (m), 3.85 (d, J=11 Hz), 4.04 (AB quartet), 4.12 (m), 4.24 (d, J=11 Hz), 4.90 (s), 4.97 (m).

### Example 36

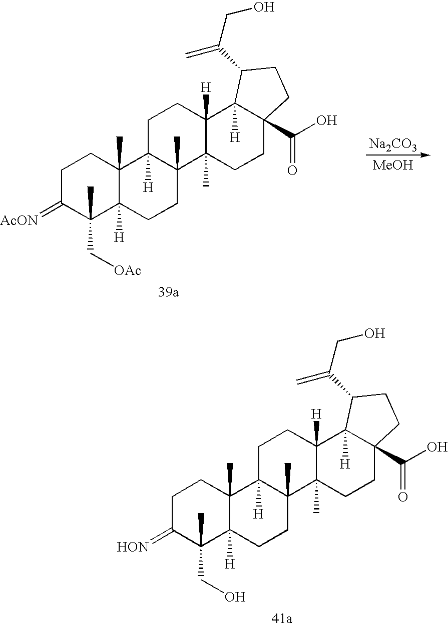

The same procedures shown above in Example 8 were employed to prepare compound 41a from 39a (407 mg, 0.69 mmol) and Na₂CO₃ (368 mg, 3.47 mmol) to afford 330 mg (95% yield) of 41a as a white powder: m.p. 225° C. (decomposes); ¹H NMR (500 MHz, CD₃OD) δ 0.9-2.1 (m), 0.94 (s), 0.96 (s), 1.00 (s), 1.02 (s), 2.26 (m), 2.90 (m), 3.42 (d, J=11 Hz), 3.61 (d, J=11.5 Hz), 4.03 (m), 4.87 (m), 4.96 (m).

### Example 37

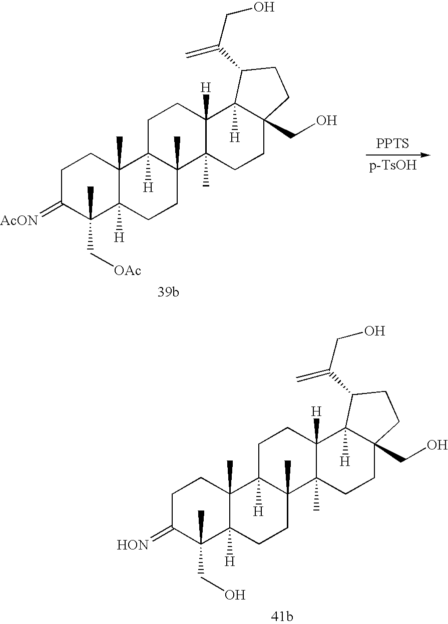

The same procedures shown above in Example 9 were employed to prepare compound 41b from 39b (200 mg, 0.35 mmol), PPTS (163 mg, 0.65 mmol) and p-TsOH monohydrate (124 g, 0.65 mmol). After purification by silica gel column chromatography eluting with 40-100% EtOAc in hexanes and 10-20% MeOH in CH₂Cl₂, 110 mg (65% yield) of 41b was obtained as a white powder: m.p. 168-178° C.; ¹H NMR (500 MHz, CD₃OD) δ 0.9-2.4 (m), 0.95 (s), 0.97 (s), 1.02 (s), 1.11 (s), 2.90 (m), 3.28 (d, J=11.5 Hz), 3.42 (d, J=11 Hz), 3.62 (d, J=11 Hz), 3.74 (d, J=11 Hz), 4.03 (m), 4.86 (m), 4.94 (m).

### Example 38

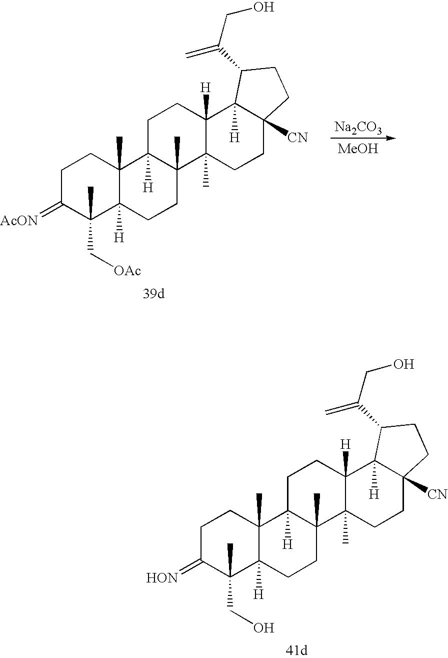

The same procedures shown above in Example 8 were employed to prepare compound 41d from 39d (117 mg, 0.20 mmol) and Na₂CO₃ (106 mg, 1.0 mmol) to afford 95 mg (100% yield) of 41d as a white solid: m.p. 230-234° C.; ¹H NMR (500 MHz, DMSO, d₆) δ 0.76-1.8 (m), 0.80 (s), 0.89 (s), 0.94 (s), 1.91 (m), 2.38 (m), 3.26 (dd, J=6 and 10 Hz), 3.39 (dd, J=5.5 and 10.5 Hz), 3.90 (m), 4.33 (t, J=6 Hz), 4.82 (t, J=5.5 Hz), 4.85 (s), 4.91 (d, J=1.5 Hz), 10.30 (s).

### Example 39

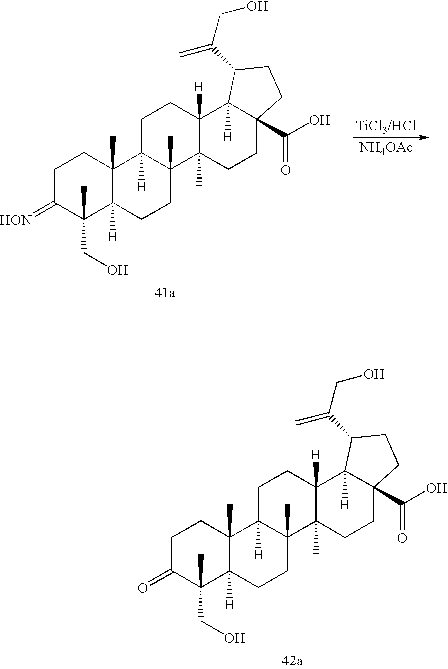

The same procedures shown above in Example 12 were employed to prepare compound 42a from 41a (100 mg, 0.199 mmol), NH₄Oac (500 mg, 6.42 mmol), and 10% TiCl₃ in 20-30% aqueous HCl (2 mL, 1.54 mmol). After purification by gradient silica gel flash column chromatography eluting with 30-50% EtOAc in hexanes and 10% MeOH in CH₂Cl₂, 50 mg (52% yield) of 42a was obtained as a white powder: m.p. 230-234° C.; ¹H NMR (500 MHz, CDCl₃) δ 0.88-2.1 (m), 0.89 (s), 0.93 (s), 1.02 (s), 1.05 (s), 2.25 (m), 2.33 (m), 2.44 (m), 2.89 (m), 3.32 (m), 3.55 (m), 3.86 (bs), 4.06 (m), 4.88 (m), 4.96 (d, J=1.5 Hz).

### Example 40

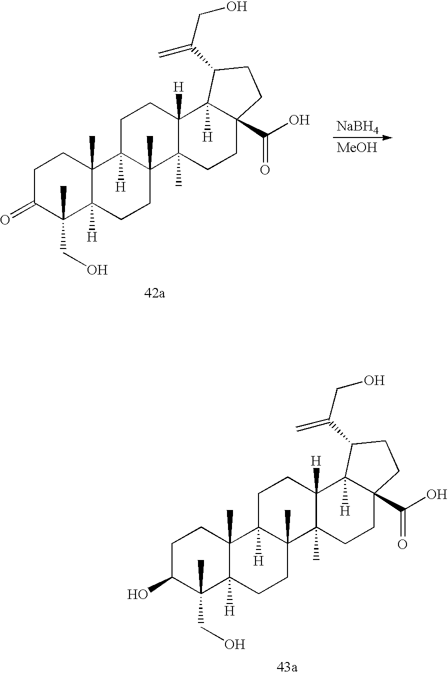

The same procedures shown above in Example 15 were employed to prepare compound 43a from 42a (40 mg, 0.08 mmol) and NaBH₄ (16 mg, 0.42 mmol). After purification by gradient silica gel column chromatography eluting with 30-50% EtOAc in hexanes and 10% MeOH in CH₂Cl₂, 25 mg (63% yield) of 43a was obtained as white solid: m.p. 258-260° C.; ¹H NMR (500 MHz, CD₃OD) δ 0.86-1.80 (m), 0.67 (s), 0.88 (s), 0.93 (s), 0.96 (s), 1.02 (s), 1.88 (m), 2.04 (m), 2.25 (m), 2.89 (m), 3.28 (d, J=11 Hz), 3.50 (d, J=10.5 Hz), 3.59 (m), 4.04 (m), 4.89 (m), 4.96 (d, J=1.5 Hz).

### Example 41

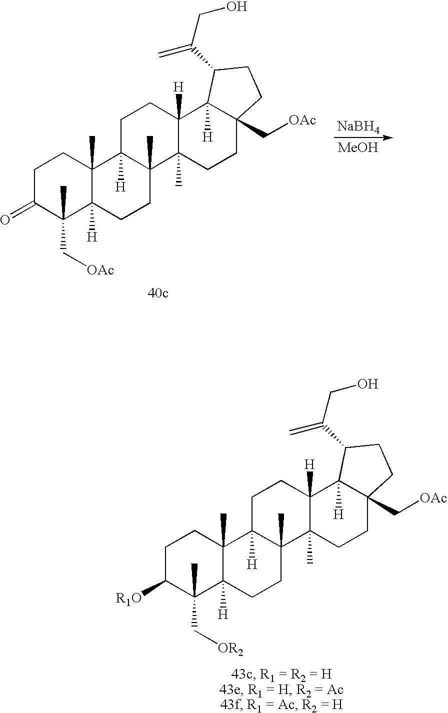

The same procedures shown above in Example 15 were employed to prepare compound 43c, 43e, and 43f from 40c (71 mg, 0.13 mmol) and NaBH₄ (10 mg, 0.26 mmol). After purification by gradient silica gel flash column chromatography eluting with 30-50% EtOAc in hexanes, 13 mg (18% yield) of 43f and 22 mg (31% yield) of 43e and 23 mg (35% yield) of 43c were obtained in the order of elution. Compound 43f: white solid with m.p. 165-168° C.; ¹H NMR (500 MHz, CDCl₃) δ 0.8-2.2 (m), 0.65 (s), 0.88 (s), 0.98 (s), 1.03 (s), 2.06 (s), 2.07 (s), 2.64 (dd, J=5.5 and 9.5 Hz), 2.89 (dd, J=5 and 12 Hz), 3.67 (dd, J=9.5 and 12 Hz), 3.84 (d, J=11 Hz), 4.11 (m), 4.23 (d, J=10 Hz), 4.86 (dd, J=5 and 12.5 Hz), 4.90 (s), 4.96 (d, J=1 Hz). Compound 43e: white solid with m.p. 90-92° C.; ¹H NMR (500 MHz, CDCl₃) δ 0.84-2.2 (m), 0.75 (s), 0.85 (s), 0.98 (s), 1.03 (s), 2.06 (s), 2.09 (s), 2.34 (m), 3.39 (m), 3.80 (d, J=11.5 Hz), 3.84 (d, J=11 Hz), 4.11 (m), 4.18 (d, J=11 Hz), 4.23 (d, J=12.5 Hz), 4.90 (s), 4.96 (d, J=1 Hz). Compound 43c: white solid with m.p. 85-90° C.; ¹H NMR (500 MHz, CD₃OD) δ 0.8-2.2 (m), 0.67 (s), 0.89 (s), 1.02 (s), 1.07 (s), 2.04 (s), 2.34 (m), 3.51 (d, J=10.5 Hz), 3.58 (dd, J=5 and 11.5 Hz), 3.84 (d, J=11 Hz), 4.04 (m), 4.37 (d, J=11 Hz), 4.87 (s), 4.96 (d, J=1.5 Hz).

### Example 42

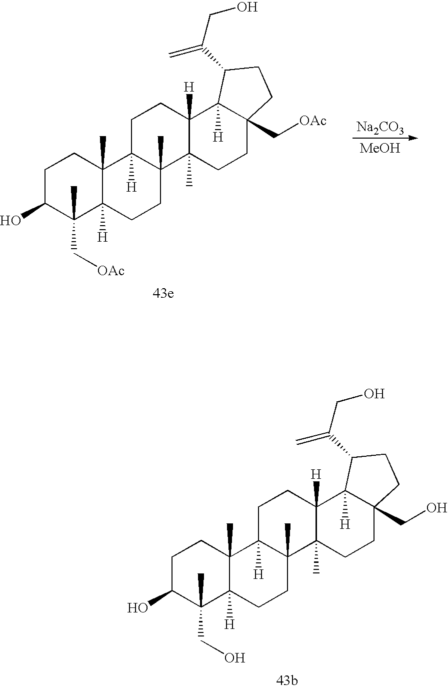

The same procedures shown above in Example 8 were employed to prepare compound 43b from 43e (13 mg, 0.02 mmol) and Na₂CO₃ (13 mg, 0.12 mmol) to afford 11 mg of 43b as a white powder.

### Example 43

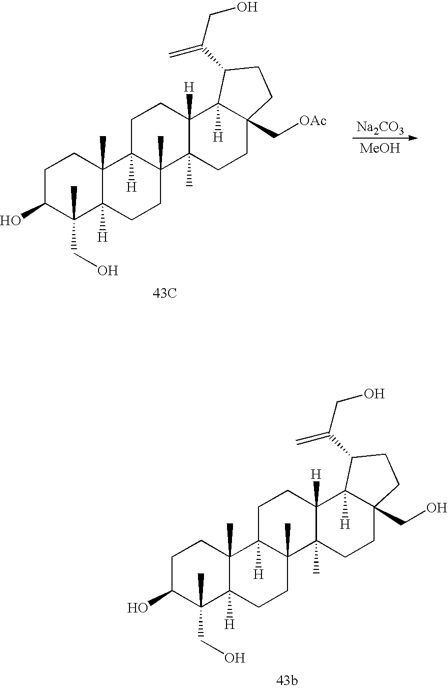

The same procedures shown above in Example 8 were employed to prepare compound 43b from 43c (12 mg, 0.02 mmol) and Na₂CO₃ (12 mg, 0.11 mmol) to afford 12 mg of 43b as a white powder.

### Example 44

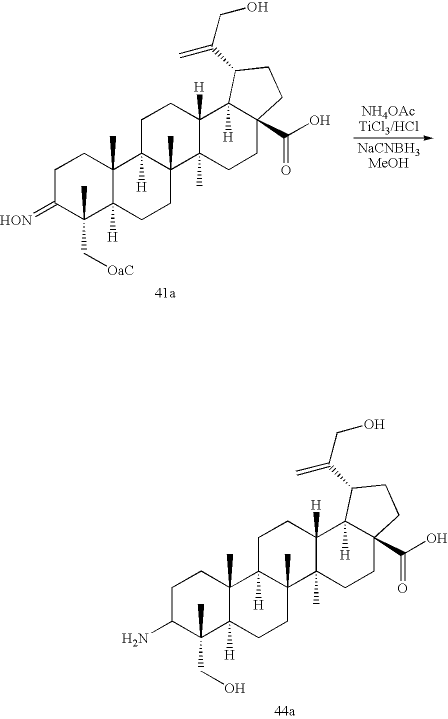

The same procedures shown above in Example 17 were employed to prepare compound 44a from 41a (200 mg, 0.40 mmol), NH₄Oac (900 mg, 11.6 mmol), NaCNBH₃ (905 mg, 14.40 mmol), and 10% TiCl₃ in 20-30% aqueous HCl (4 mL, 3.10 mmol). After purification by gradient silica gel flash column chromatography eluting with 10-20% MeOH in CH₂Cl₂, 30 mg (15% yield) of 44a was obtained as a white solid: m.p. >299° C.; ¹H NMR (500 MHz, CD₃OD) δ 0.90-2.2 (m), 0.92 (s), 0.95 (s), 0.99 (s), 1.01 (s), 2.24 (m), 2.37 (m), 2.93 (m), 3.16 (m), 3.41 (d, J=11 Hz), 3.65 (d, J=10.5 Hz), 4.04 (m), 4.06 (m), 4.86 (s), 4.95 (m). Negative ESI-MS, m/e 486.3 (M−H⁺).

### Example 45

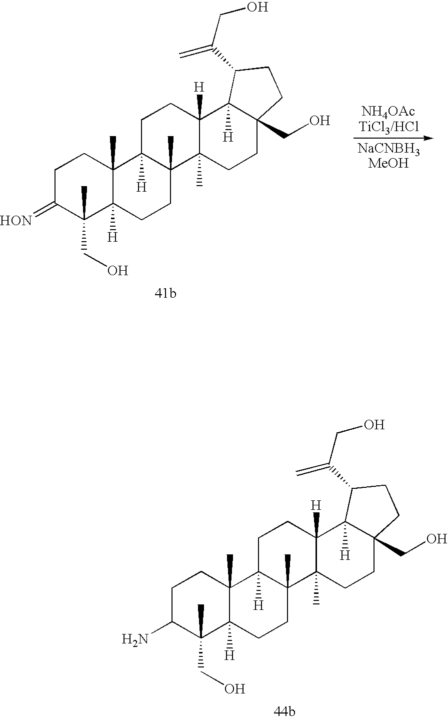

The same procedures shown above in Example 17 were employed to prepare compound 44b from 41b (90 mg, 0.184 mmol), NH₄Oac (300 mg, 3.85 mmol), NaCNBH₃ (300 mg, 4.77 mmol), and 10% TiCl₃ in 20-30% aqueous HCl (0.9 mL, 0.73 mmol). After purification by gradient silica gel flash column chromatography eluting with 10:90:1 to 20:80:1 of MeOH:CH₂Cl₂:Conc. NH₄OH, 30 mg (35% yield) of 44b was obtained as a white powder: m.p. >299° C.; ¹H NMR (500 MHz, CD₃OD) δ 0.8-2.0 (m), 0.83 (s), 0.90 (s), 1.02 (s), 1.08 (s), 2.11 (m), 2.30 (m), 2.92 (dd, J=5 and 11.5 Hz), 3.27 (d, J=11 Hz), 3.37 (d, J=11 Hz), 3.55 (d, J=11 Hz), 3.72 (d, J=11 Hz), 4.03 (m), 4.86 (m), 4.94 (m).

### Example 46

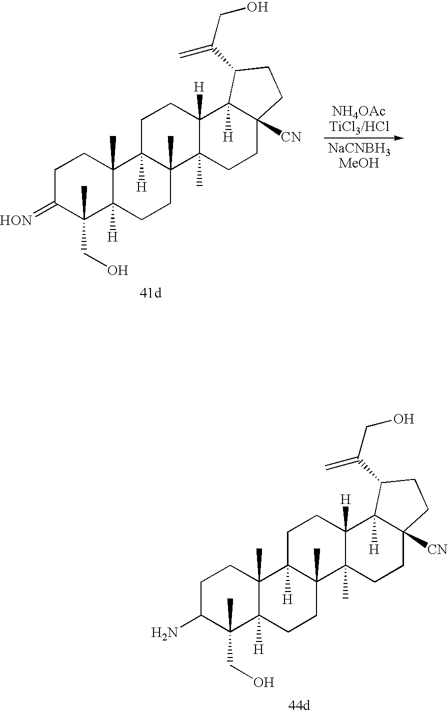

The same procedures shown above in Example 17 were employed to prepare compound 44d from 41d (91 mg, 0.18 mmol), NH₄Oac (0.5 g, 6.42 mmol), NaCNBH₃ (0.5 g, 7.95 mmol), 10% TiCl₃ in 20-30% aqueous HCl (2 mL, 1.55 mmol) to afford 126 mg of 44d as a white solid: m.p. >299° C.; ¹H NMR (500 MHz, CD₃OD) δ 0.86-2.1 (m), 0.94 (s), 0.96 (s), 1.01 (s), 1.11 (s), 2.26 (m), 2.52 (m), 3.18 (dd, J=5 and 12 Hz), 3.42 (d, J=11 Hz), 3.66 (d, J=10.5 Hz), 4.05 (s), 4.92 (m), 5.01 (m).

### Example 47

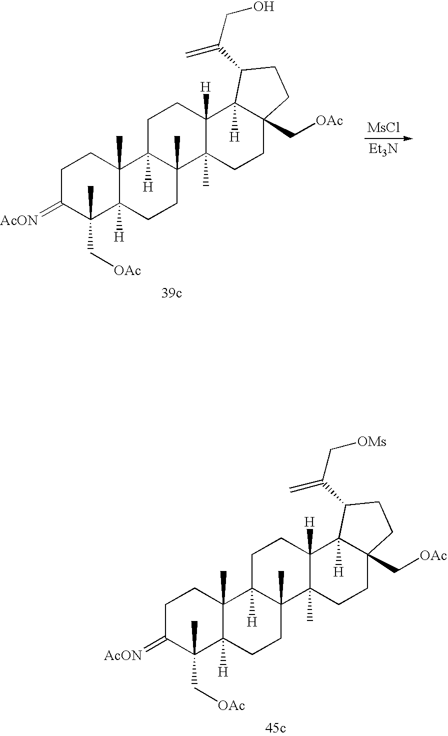

To a stirred solution of 39c (262 mg, 0.43 mmol) and Net₃ (0.2 mL, 1.43 mmol) in ether (6 mL) at 0° C. under N₂ was added dropwise CH₃SO₂Cl (0.04 mL, 0.51 mmol). The solution turned light yellow and solids (NEt₃.HCl) were formed. After 15 min, the reaction mixture was diluted with EtOAc (30 mL) and washed with H₂O (30 mL). The organic layer was separated, dried (Na₂SO₄) and concentrated under reduced pressure to a residue which was purified by silica gel column chromatography eluting with 30-50% EtOAc in hexanes to obtain 146 mg (49% yield) of 45c as a white solid: m.p. 91-93° C.; ¹H NMR (500 MHz, CDCl₃) δ 0.8-2.3 (m), 0.88 (s), 0.99 (s), 1.06 (s), 1.16 (s), 2.05 (s), 2.07 (s), 2.17 (s), 2.42 (m), 2.59 (m), 2.67 (m), 3.04 (s), 3.83 (d, J=11 Hz), 4.10 (d, J=11 Hz), 4.17 (d, J=10.5 Hz), 4.24 (d, J=11 Hz), 4.67 (m), 5.09 (s), 5.10 (s).

### Example 48

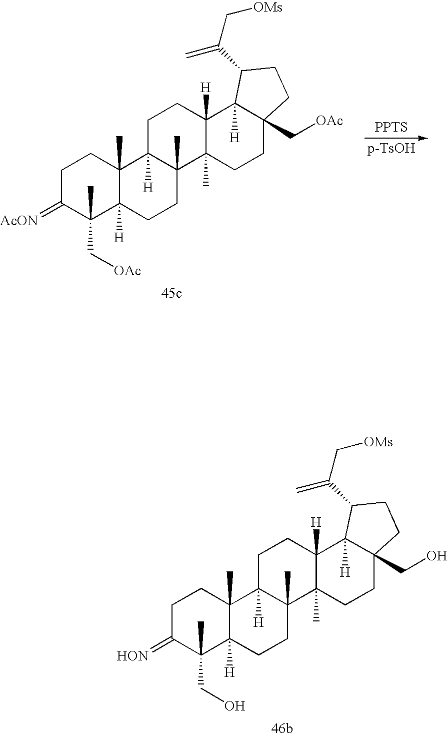

The same procedures shown above in Example 9 were employed to prepare compound 46b from 45c (400 mg, 0.06 mmol), PPTS (300 mg, 1.19 mmol) and p-TsOH monohydrate (300 mg, 1.57 mmol) to afford 300 mg (86% yield) of 46b.

### Example 49

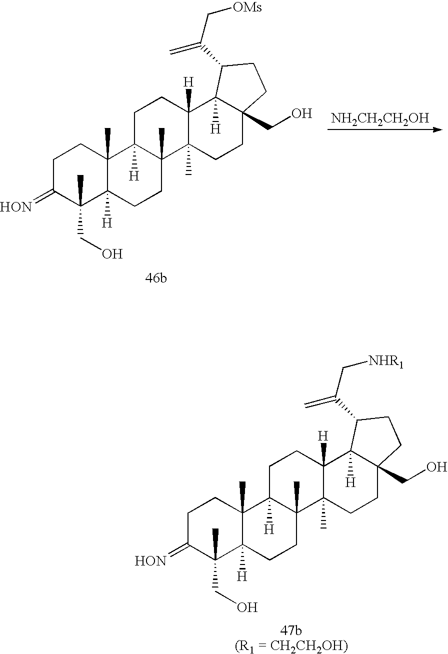

A solution of 46b (0.3 g, 0.53 mmol) and ethanolamine (10 mL) was stirred at rt for 16 h. The reaction mixture was diluted with EtOAc (30 mL) and MeOH (1 mL) and washed with H₂O (10 mL). The organic layer was separated, dried (Na₂SO₄) and concentrated under reduced pressure to a residue which was purified by silica gel column chromatography eluting with 10:90:1 to 20:80:1 of MeOH:CH₂Cl₂:Conc. NH₄OH to afford 184 mg (56% yield) of 47b (R₁=CH₂CH₂OH) as a white solid: m.p. 208-210° C.; ¹H NMR (500 MHz, CD₃OD) δ 0.9-2.02 (m), 0.95 (s), 0.97 (s), 1.03 (s), 1.12 (s), 2.14 (m), 2.24 (m), 2.38 (m), 2.91 (m), 3.39 (m), 3.62 (d, J=11 Hz), 3.74 (t, J=5.5 Hz), 4.91 (m), 5.05 (s). Positive ESI-MS, m/e 531 (M+H⁺).

### Example 50

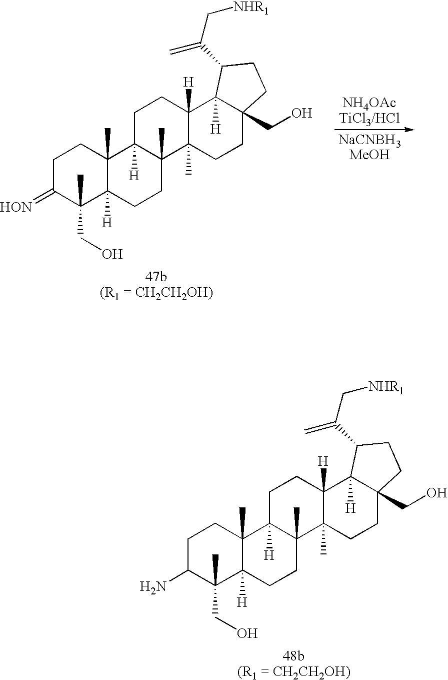

The same procedures shown above in Example 17 were employed to prepare compound 48b from 47b (98 mg, 0.18 mmol), NH₄Oac (400 mg, 5.14 mmol), NaCNBH₃ (380 mg, 6.04 mmol), and 10% TiCl₃ in 20-30% aqueous HCl (1.2 mL, 0.93 mmol) After purification by gradient silica gel flash column chromatography eluting with 10:90:1 to 20:80:1 of MeOH:CH₂Cl₂:Conc. NH₄OH, 20 mg of 48b (R₁=CH₂CH₂OH) was obtained as a white powder: m.p. >299° C.; ¹H NMR (500 MHz, CD₃OD) δ 0.9-2.02 (m), 0.94 (s), 0.97 (s), 1.05 (s), 1.10 (s), 2.15 (m), 2.40 (m), 2.94 (m), 3.20 (t, J=5.5 Hz), 3.44 (d, J=11 Hz), 3.68 (d, J=11 Hz), 3.74 (d, J=11 Hz), 3.84 (t, J=5.5 Hz), 3.90 (m), 4.79 (m), 5.03 (m), 5.24 (m). Positive ESI-MS, m/e 501.3, 517.3, 531.5 and 545.3 (M+H⁺).

### Example 51

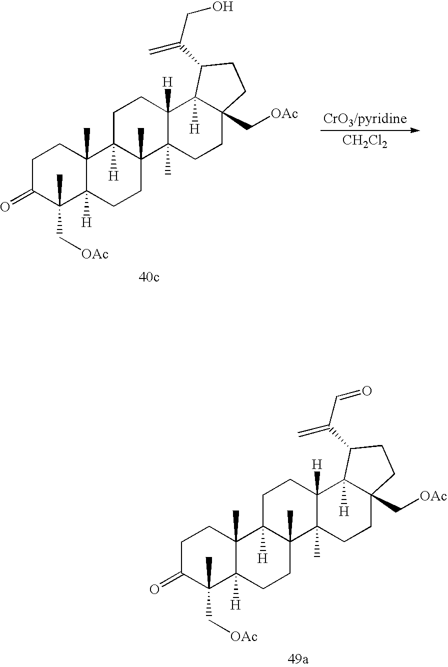

The same procedures shown above in Example 21 were employed to prepare compound 49a from 40c (200 mg, 0.36 mmol), pyridine (0.35 mL, 4.3 mmol) and CrO₃ (216 mg, 2.1 mmol). After purification by silica gel column chromatography eluting with 20-50% EtOAc in hexanes, 124 mg (62% yield) of 49a was obtained as white solid: m.p. 95-99° C.; ¹H NMR (500 MHz, CDCl₃) δ 0.8-2.0 (m), 0.93 (s), 0.96 (s), 0.99 (s), 1.07 (s), 2.02 (s), 2.07 (s), 2.16 (m), 2.45 (m), 2.81 (m), 3.87 (d, J=11 Hz), 4.04 (AB quartet), 4.28 (d, J=11 Hz), 5.92 (m), 6.27 (s), 9.51 (s).

### Example 52

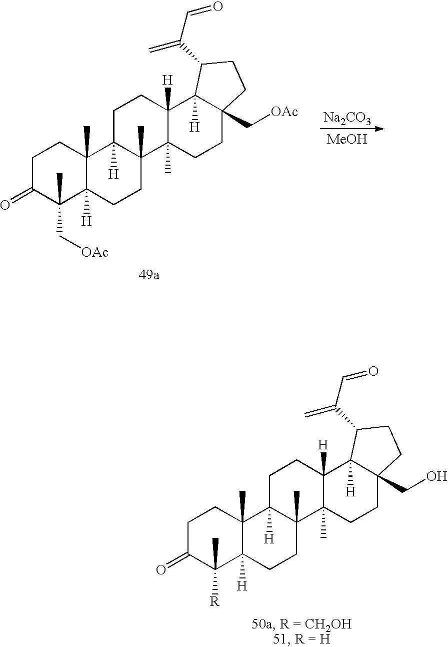

The same procedures shown above in Example 8 were employed to deacetylate 49a (97 mg, 0.17 mmol) with Na₂CO₃ (93 mg, 0.85 mmol) in MeOH (10 mL). After purification by silica gel flash column chromatography eluting with 30-50% EtOAc in hexanes), 24 mg (32% yield) of 51 and 25 mg (31% yield) of 50a were obtained in the order of elution. Compound 50a: white solid with m.p. 228-231° C.; ¹H NMR (500 MHz, CDCl₃) δ 0.9-2.4 (m), 0.96 (s), 0.99 (s), 1.03 (s), 1.07 (s), 2.61 (m), 2.78 (m), 3.40 (m), 3.64 (dd, J=7 and 11.5 Hz), 3.80 (dd, J=5.5 and 11.5 Hz), 5.93 (m), 6.29 (s), 9.51 (s). Compound 51: white solid with m.p. 192-196° C.; ¹H NMR (500 MHz, CDCl₃) δ 0.8-2.1 (m), 0.94 (s), 0.97 (d, J=6.5 Hz), 1.00 (s), 1.06 (s), 2.18 (m), 2.27 (m), 2.41 (m), 2.77 (m), 3.38 (dd, J=5 and 11 Hz), 3.80 (dd, J=4 and 10.5 Hz), 5.93 (m), 6.28 (s), 9.51 (s).

### Example 52

### A. Biological Assays

### MTS Cell Viability Assay

Cells were plated, the evening before treatment, for each treatment on 96-well plates (1×10⁴ cells/well) in 100 μL volume per well. Analog solutions were prepared by diluting each test compound in DMSO (10 mM) with the appropriate cell growth media for each individual cell line, or in “Universal Media” if several cell lines were to be assayed in parallel, immediately prior to cell treatment. Universal Media consisted of 5 mL sodium pyruvate (100× liquid stock, MediaTech), 5 mL glucose (100×, 45% liquid stock, MediaTech), 5 mL Penicillin/Streptomycin (100× liquid stock, MediaTech), 10 mL sodium bicarbonate (50× liquid stock, MediaTech), 25 mL Fetal Calf Serum, 1.25 mL insulin (4 mg/mL, Gibco) and 449 mL RPMI media with 2 mM L-glutamine for a total volume of 500 mL.

Cells were treated by aspirating plating media from each well and adding 80 μL of betulinic acid (control) or each analog solution in serial dilutions to the adherent cells. All treatments were performed in triplicate. Growth media (80 μL) was added to 3 blank wells (no cells) to measure background from the growth media. Growth media alone (no DMSO or test compound) was added to 3 wells containing cells to measure the baseline MTS activity. Vehicle (DMSO) control in serial dilutions, added to cells, was also included to monitor basal toxicity from DMSO. Cells were incubated at 37° C. for 72 h, or 120 h for the OVCAR cell lines. MTS reagent (per 96-well plate) were prepared by combining 2 mL of MTS working solution (Cell Titer Aqueous Non-Radioactive Cell Proliferation Assay, Promega, cat#G1112), 100 μL of 0.92 mg/mL phenazine methosulfate/Dulbecco's PBS and 2.1 mL growth media. MTS reagent (40 μL) was added to each well and incubated at 37° C. for 1.5 to 4 h. Plates were gently shaken by hand until solution in each well appeared homogenous. Absorbances at 490 nm were measured on a Wallac Victor II plate reader at multiple time points following the addition of MTS reagent for each plate. Triplicate absorbance (490 nm) measurements were averaged following background (no cell) subtraction for each drug concentration. Percent Cell Viability was calculated for each drug concentration using the following equation:

{[Absorbance(analog treated)]/[Absorbance(DMSO treated)]}×100%

Percent viability (y-axis) was plotted against drug concentration (x-axis) and the resulting graph was used to determine the 50% inhibitory concentration (IC₅₀) for each drug.

### Caspase Assay

Cells were plated, the evening before treatment, for each treatment (1×10⁴ cells/well) on black-walled, clear-bottomed, 96-well plate, in 100 μL volume per well. Analog solutions were prepared immediately prior to cell treatment by diluting each test compound in the appropriate cell growth media without fetal calf serum (FCS) or “Universal Media” without FCS if several cell lines were to be assayed in parallel.

Cells were treated by aspirating plating media from each well and adding 70 μL of betulinic acid (control) or each analog solution to the adherent cells in serial dilutions. All treatments were performed in duplicate. Growth media (70 μL) was added to 2 blank wells (no cells) to measure background from the growth media. Growth media alone (no DMSO or test compound) was added to 2 wells containing cells to measure the baseline fluorescence. Vehicle (DMSO) control serial dilutions, added to cells, were also included to monitor basal caspase induction from DMSO. Cells were incubated at 37° C. for 24 h. Caspase assay reagent (per 96-well plate) was prepared according to manufacturer's instructions (Homogeneous Caspases Assay, fluorometric, Roche) by combining 6.3 mL Incubation Buffer with 0.7 mL of Substrate Stock Solution. Caspase assay reagent (70 μL) was added to each well; the plate was gently shaken by hand for 15-20 seconds and incubated at 37° C. for 4 h.

Fluorescent emission was measured at 535 nm on a plate reader using the “homogeneous caspase” program (excitation wavelength=490 nm, emission wavelength=535 nm). Duplicate wells for each treatment were averaged following background (no cells) subtraction (emission 535 nm value from all experimental emission 535 nm values) for each analog concentration. Percent change in caspase activity was calculated for each analog concentration using the following equation:

{{[Emission₅₃₅(analog treated)]−[Emission₅₃₅(DMSO treated)]}/Emission₅₃₅(DMSO treated)}×100%

DMSO treatment represented baseline caspase activation in the absence of analog. The percent change in caspase activity was plotted on the y-axis for each analog treatment.

### Annexin-V Assay

Cells were plated (8.75×10⁵ cells/6 cm. diameter tissue culture plates), the evening before treatment, in 3 mL volume per plate. This cell density is equivalent to the cell density used in the MTS and caspase assays (1×10⁴ cells/well of a 96-well plate).

Analog solutions were prepared, immediately prior to cell treatment, in the same as described for caspase assay.

Cells were treated by aspirating plating media from each well and adding 3 mL of betulinic acid (control) or each analog solution to the adherent cells in serial dilutions. Growth media alone (no DMSO or drug) was added to a plate containing cells to measure the baseline Annexin-V reactivity. Vehicle (DMSO) alone control serial dilutions were also prepared to monitor Annexin-V reactivity from DMSO. Cells were incubated at 37° C. for 6 h. Treatment media (3 mL) was removed from each plate and added to a 15 mL conical tube containing 0.333 mL FCS (final FCS concentration of 10%). The treatment media was saved to include any apoptotic/dead cells that may have detached from the plate during analog treatment. FCS was added to the recovered media to prevent further cell damage and improve the efficiency of cell pelleting during subsequent centrifugation steps (empirical observation). Adherent cells were rinsed once with PBS and 1 mL of trypsin was added. Plates were rotated several times to assure coating of the entire surface with trypsin, which was then removed. Plates were incubated at 37° C. for 4-5 min. Trypsinized cells were re-suspended in the recovered media for each sample. Cell suspension was placed back into 15 mL tubes, which was then cooled on ice. Cells were re-suspended by pipetting 7-8 times. The tubes were centrifuged at 130×g for 5 min at 4° C. The resulting cell pellets were re-suspended in 1 mL of ice cold 1× Nexin Buffer (Guava Nexin kit, Guava Technologies) and transferred to 1.5 mL conical microcentrifuge tubes to rinse any residual growth media. This procedure was followed by centrifugation of cells at 130×g for 5 min, at 4° C. Re-suspension of the resulting cell pellet in 50 μL Nexin Staining Solution (Guava Nexin kit, Guava Technologies) was followed by incubation on ice, in the dark, for 20 min. Nexin samples were analyzed immediately on the Guava flow cytometer, using the Guava Nexin software package (see Guava user's manual and Guava Nexin kit protocol on data acquisition and analysis protocols). Live cell, apoptotic cell and necrotic cell populations are differentiated by the permeability of Annexin V-PE and/or 7-AAD.

### B. Biological Results

Cytotoxicity dose response for the synthesized triterpenoid derivatives in SK-MEL-2 (melanoma), A-375 (melanoma), Daoy (glioblastoma), LN-229 (glioblastoma), OVCAR-3 (ovarian carcinoma), HT-29 (colon carcinoma), MCF-7 (breast carcinoma) cell lines using the standard MTS assay (described above) is summarized in Table 1.

TABLE 1
Biological Activities of Selected 23-HBA Analogs (IC₅₀ values in μM)

| Compound | SK-MEL-2 | A-375 | Daoy | LN-229 | OVCAR-3 | HT-29 | MCF-7 |
|---|---|---|---|---|---|---|---|
| 1 | >75 | — | >75 | — | — | — | — |
| 2 | 33 | 51 | 31 | 61 | 59 | 37 | 50 |
| 4 | 33 | — | 59 | — | — | — | — |
| 5 | >75 | — | 65 | — | — | — | — |
| 12 | 38 | — | 25 | — | — | — | — |
| 15b | 35 | — | 34 | — | — | — | — |
| 15c | 34 | — | 61 | — | — | — | — |
| 17c | 16 | — | 16 | — | — | — | — |
| 18b | >75 | — | 34 | — | — | — | — |
| 18c | 29 | — | 16 | — | — | — | — |
| 22 | 36 | — | 38 | — | — | — | — |
| 23 | 2 | 5 | 2 | 15 | — | 6 | 5 |
| 25 | 36 | — | 38 | — | — | — | — |
| 27 | 64 | — | >75 | — | — | — | — |
| 29 | 16 | — | 14 | — | — | — | — |
| 30 | 3 | 4 | 5 | 16 | — | 13 | <2 |
| 31 | 3 | 14 | <2 | 16 | — | 4 | 17 |
| 33 (R═CH₂CH₂OH) | 16 | — | 17 | — | — | — | — |
| 34 (R═CH₂CH₂OH) | 2 | <2 | <2 | 2 | — | 2 | <2 |
| 35 | 24 | — | 31 | — | — | — | — |
| 37 | 14 | — | 17 | — | — | — | — |
| 38 | 18 | — | 31 | — | — | — | — |
| 39c | 36 | — | 35 | — | — | — | — |
| 39d | 14 | — | 33 | — | — | — | — |
| 40c | 33 | — | 56 | — | — | — | — |
| 43a | >75 | — | >75 | — | — | — | — |
| 43c | 17 | — | 38 | — | — | — | — |
| 43e | 16 | — | 16 | — | — | — | — |
| 43f | 16 | — | 16 | — | — | — | — |
| 44a | >75 | — | >75 | — | — | — | — |
| 44b | 17 | — | 18 | — | — | — | — |
| 44d | 32 | — | 73 | — | — | — | — |
| 47b | 38 | — | 38 | — | — | — | — |
| 49a | 5 | 5 | 9 | 15 | — | 5 | 4 |
| 50a | 16 | — | 16 | — | — | — | — |
| 51 | 16 | — | 16 | — | — | — | — |

### REFERENCES

1. Ikuta, A.; Itokawa, H. Triterpenoids of Paeonia japonica callus tissue. Phytochem. 1988, 27, 2813-2815.
2. Ye, W.-C.; Ji, N.-N.; Zhao, S.-X.; Liu, J.-H., Ye, T.; McKeverey, M. A.; Stevenson, P. Triterpenoids from Pulsatilla chinensis. Phytochem. 1996, 42, 799-802.
3. Eiznhamer, D. A.; Xu, Z.-Q. Betulinic acid, a naturally-occurring inducer of apoptosis, is a promising anticancer candidate. Idrugs 2004, 7, 359-373.
4. Ye, W.-C.; Zhang, Q. W.; Hsiao, W. L. W.; Zhao, S.-X.; Che, C.-T. Novel cytotoxic upane-type glycosides from Pulsatilla Chinensis. Planta Med. 2002, 68, 183-186.
5. Liu, W.-K.; Ho, J. C. K.; Cheung, F. W. K.; Liu, B. P. L.; Ye, W.-C.; Che, C.-T. Apoptotic activity of betulinic acid derivatives on murine melanoma B16 cell line. Eur J Pharmacol. 2004, 498, 71-78.
6. Ji, Z.-N.; Ye, W.-C.; Liu, G.-G.; Hsiao, W. L. W. 23-Hydroxybetulinic acid-mediated apoptosis is accompanied by decreases in bcl-2 expression and telomerase activity in HL-60 cells. Life Sciences, 2002, 72, 1-9.
7. Zheng, Y.; Wang, G.; Wu, X.; Sun, J.; Li, X.; Gu, Y.; Lv, H.; Liang, Y.; Yan, B.; Ye, W. Quantitative analysis of 23-hydroxybetulinic acid, a novel anticancer substance, in the cellular environment of a human colon adenocarcinoma cell line for cellular pharmacokinetic studies. Rapid Commun. Mass Spectrom. 2007, 21, 779-786.
8. Yook, C.-S.; Liu, X.-Q.; Chang, S.-Y.; Park, S.-Y.; Nohara, T. Lupane-triterpene glycosides from the leaves of Acanthopanax gracilistylus. Chem. Pharm. Bull. 2002, 50, 1383-1385.
9. Nishimura, K.; Fukuda, T.; Miyase, T.; Noguchi, H.; Chen, X.-M. Activity-guided isolation of triterpenoid Acyl CoA cholesteryl acyl transferase (ACAT) inhibitors from Ilex kudincha. J. Nat. Prod. 1999, 62, 1061-1064.
10. Gao, H.; Wu, L.; Kuroyanagi, M.; Harada, K.; Kawahara, N.; Nakane, T.; Umehara, K.; Hirasawa, A.; Nakamura, Y. Antitumor-promoting constituents from Chaenome sinensis KOEHNE and their activities in JB16 mouse epidermal cells. Chem. Pharm. Bull. 2003, 51, 1318-1321.
11. Monkhe, T.; Mulholland, D.; Nicholls, G. Triterpenoids from Bersama swinnyi. Phytochem. 1998, 49, 1819-1820.
12. Adam, G.; Lischewski, M.; Phiet, H. V., Preiss, A.; Schmidt, J.; Sung, T. V. 3α-Hydroxy-lup-20(29)-ene-23,28-dioic acid from Schefflera octophylla. Phytochem. 1982, 21, 1385-1387.
13. Zhou, J. P.; Li D.; Wu, X. M.; Ye, W. C.; Zhang, L. Y. Synthesis and antitumor activity of derivatives of 23-hydroxybetulinic acid. Chinese Chem. Lett. 2007, 18, 1195-1198.
14. Bi, Y.; Xu, J.; Wu, X.; Ye, W.; Yuan, S.; Zhang, L. Synthesis and cytotoxic activity of 17-carboxylic acid modified 23-hydroxy betulinic acid ester derivatives. Bioorg. Med. Chem. Lett. 2007, 17, 1475-1478.
15. (a) Baldwin, J. E.; Jones, R. H.; Najera, C.; Yus, M. Functionalisation of unactivated methyl groups through cyclopalladation reactions. Tetrahedron, 1985, 41, 699-711. (b) Peakman, T. M.; Haven, H. Lo ten; Rullkotter, J.; Curiale, J. A. Characterization of 24-nor-triterpenoids occurring in sediments and crude oils by comparison with synthesized standards. Tetrahedron, 1991, 47, 3779-3786.

What is claimed is:

1. A compound of the formula 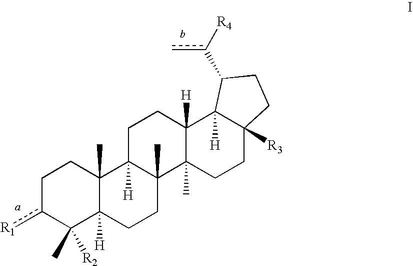
or a pharmaceutically acceptable salt thereof, wherein
each [symbol] is independently a single or double bond;
when bond a is a double bond, R₁ is ═O, ═S, ═N—OH, ═N—OR₅, or ═N—OCOR₅; or when bond a is a single bond, R₁ is H, halogen, NH₂, OH, SH, NHOH, NHR₅, NH(CH₂)ₙX, NR₅R₆, OR₅, OCOR₅, OCO(CH₂)ₙX, OC(O)OR₅, OCO(HC═CH)ₙR₅, OC(O)NHR₅, OC(O)NR₅R₆, OSO₂H, OSO₃H, OSO₂(R₅), OSO₂(CH₂)ₙX, OSi(R₅)ₘ(R₆)₃₋ₘ, SR₅, SCOR₅, SCO(CH₂)ₙX, SC(O)NH₂, SC(O)NHR₅, SC(O)NR₅R₆, NHCOR₅, NHC(O)OR₅, N(R₆)C(O)OR₅, NHC(O)NH₂, NHC(O)NHR₅, NHC(O)NR₅R₆, N(R₆)C(O)NH₂, N(R₆)C(O)NHR₅, N(R₆)C(O)NR₅R₆;
R₂ and R₃ are independently selected from CH₂NH₂, CH₂OH, CH₂SH, CH₂SePh, CH₂X, CH₂NHOH, CN, CHO, CO₂H, CH₂NHOR₅, CH═N—OH, CH═N—OR₅, CH═N—OCOR₅, CH₂NHR₅, CH₂NH(CH₂)ₙX, CH₂NR₅R₆, CH₂OR₅, CH₂OCOR₅, CH₂OC(O)OR₅, CH₂OCO(CH₂)ₙX, CH₂OCO(HC═CH)ₙR₅, CH₂OC(O)NH₂, CH₂OC(O)NHR₅, CH₂OC(O)NR₅R₆, CH₂OSO₂(R₅), CH₂OSO₂(CH₂)ₙX, CH₂OSi(R₅)ₘ(R₆)₃₋ₘ, CH₂SR₅, CH₂SCOR₅, CH₂SCO(CH₂)ₙX, CH₂SC(O)NH₂, CH₂SC(O)NHR₅, CH₂SC(O)NR₅R₆, CH₂NHCOR₅, CH₂NHC(O)OR₅, CH₂NHC(O)NH₂, CH₂NHC(O)NHR₅, CH₂NHC(O)NR₅R₆, CH₂N(R₆)COR₅, CH₂N(R₆)C(O)OR₅, CH₂N(R₆)C(O)NH₂, CH₂N(R₆)C(O)NHR₅, CH₂N(R₆)C(O)NR₅R₆, CH(OR₅)₂, CH(OH)OR₅, CO₂R₅, C(O)NH₂, C(O)NHR₅, C(O)NR₅R₆, C(O)NHOH, or C(O)NHOR₅;
R₄ is selected from CH₃, CH₂NH₂, CH₂OH, CH₂SH, CH₂SePh, CH₂X, CH₂NHOH, CN, CHO, CO₂H, CH₂NHOR₅, CH═N—OH, CH═N—OR₅, CH═N—OCOR₅, CH₂NHR₅, CH₂NH(CH₂)ₙX, CH₂NR₅R₆, CH₂OR₅, CH₂OCOR₅, CH₂OC(O)OR₅, CH₂OCO(CH₂)ₙX, CH₂OCO(HC═CH)ₙR₅, CH₂OC(O)NH₂, CH₂OC(O)NHR₅, CH₂OC(O)NR₅R₆, CH₂OSO₂(R₅), CH₂OSO₂(CH₂)ₙX, CH₂OSi(R₅)ₘ(R₆)₃₋ₘ, CH₂SR₅, CH₂SCOR₅, CH₂SCO(CH₂)ₙX, CH₂SC(O)NH₂, CH₂SC(O)NHR₅, CH₂SC(O)NR₅R₆, CH₂NHCOR₅, CH₂NHC(O)OR₅, CH₂NHC(O)NH₂, CH₂NHC(O)NHR₅, CH₂NHC(O)NR₅R₆, CH₂N(R₆)COR₅, CH₂N(R₆)C(O)OR₅, CH₂N(R₆)C(O)NH₂, CH₂N(R₆)C(O)NHR₅, CH₂N(R₆)C(O)NR₅R₆, CH(OR₅)₂, CH(OH)OR₅, CO₂R₅, C(O)NH₂, C(O)NHR₅, C(O)NR₅R₆, C(O)NHOH, or C(O)NHOR₅;
R₅ and R₆ are independently a straight or branched C₁₋₈ alkyl, aryl-C₁₋₈ alkyl, heterocycle-C₁₋₈ alkyl, cyclo(C₃₋₉)alkyl, aryl, or heterocycle, wherein
the aryl comprises phenyl or a polycyclic aryl group such as naphthyl;
the heterocyle comprises
a five or six membered aromatic heterocycle such as pyridyl, diazinyl, pyrimidinyl, 5-methoxy pyrimidinyl, (1,2,4)triazine-3,5-dione-6-yl, 6-mercaptopyrimidine-4-yl, pyrrolyl, pyrazolyl, imidazolyl, oxazolyl, isoxazolyl, thiazolyl, isothiazolyl, furanyl, or thienyl;
a 3 to 9 membered non-aromatic heterocycle comprising piperazinyl, 4-methyl piperazinyl, pyrrolidinyl, imidazolidinyl, imidazolinyl, thiazolidinyl, thiazolinyl, isothiazolidinyl, isothiazolinyl, pyranyl, or morpholinyl; or
a polycyclic heterocycle such as indolyl, benzthienyl, benzofuranyl, isoindolyl, isobenzothienyl, or isobenzofuranyl;
wherein each alkyl, cycloalkyl, aryl, and heterocycle are each optionally substituted with one or more groups (e.g., 1, 2, or 3 group) which are each independently C₁₋₈ alkyl, C₁₋₈ alkoxy, C₁₋₈ alkylamino, di(C₁₋₈ alkyl)amino, C₁₋₈ alkylamino-C₁₋₈ alkyl, di(C₁₋₆ alkyl)amino-C₁₋₈ alkyl, OC(O)OR₇, OC(O)N(R₇)₂, C(O)OR₇, C(O)N(R₇)₂, S(O)₂R₇, S(O)₂N(R₇)₂, N(R₇)C(O)N(R₇)₂, N(R₇)C(O)OR₇, CN, N₃, NHOH, ═NOH, NH₂, NO₂, OH, SH, F, Cl, Br, or I, wherein each R₇ is independently H or C₁₋₈ alkyl; or
R₅ and R₆ taken together with the carbon atoms to which they are attached form a cyclo(C₃₋₉)alkyl, aryl, or heterocycle, wherein
the aryl comprises a phenyl or a polycyclic aryl group;
the heterocyle comprises
a five or six membered aromatic heterocycle, such as pyridyl, diazinyl, pyrimidinyl, 5-methoxy pyrimidinyl, (1,2,4)triazine-3,5-dione-6-yl, 6-mercaptopyrimidine-4-yl, pyrrolyl, pyrazolyl, imidazolyl, oxazolyl, isoxazolyl, thiazolyl, isothiazolyl, furanyl, or thienyl;
a 3 to 9 membered non-aromatic heterocycle comprising piperazinyl, pyrrolidinyl, imidazolidinyl, imidazolinyl, thiazolidinyl, thiazolinyl, isothiazolidinyl, isothiazolinyl, 4-methyl piperazinyl, pyranyl, or morpholinyl; or
a polycyclic heterocycle such as indolyl, benzthienyl, benzofuranyl, isoindolyl, isobenzothienyl, or isobenzofuranyl;
wherein the cycloalkyl, aryl, and heterocycle are each optionally substituted with one or more groups (e.g., 1, 2, or 3 groups) which are each independently C₁₋₈ alkyl, C₁₋₈ alkoxy, C₁₋₈ alkylamino, di(C₁₋₈ alkyl)amino, C₁₋₈ alkylamino-C₁₋₈ alkyl, di(C₁₋₆ alkyl)amino-C₁₋₈ alkyl, OC(O)OR₇, OC(O)N(R₇)₂, C(O)OR₇, C(O)N(R₇)₂, S(O)₂R₇, S(O)₂N(R₇)₂, N(R₇)C(O)N(R₇)₂, N(R₇)C(O)OR₇, CN, N₃, NHOH, ═NOH, NH₂, NO₂, OH, SH, F, Cl, Br, or I wherein each R₇ is independently H or C₁₋₈ alkyl;
each X is independently Y or aryl, heterocycle, or cyclo(C₃₋₉)alkyl, wherein the aryl, heterocycle, or cyclo(C₃₋₉)alkyl are each optionally substituted with one or more Y groups (e.g., 1, 2, or 3 Y groups), wherein each Y is independently F, Cl, Br, I, CN, N₃, NHOH, ═NOH, NH₂, OH, SH, NHR₅, NR₅R₆, OR₅, SR₅, CO₂H, CO₂R₅, SO(OH)₂, or SO(OR₅)₂;
each m is independently 0, 1, 2, or 3;
each n is independently 1, 2, 3, 4, or 5;
with the proviso that:
when bond a is a single bond, bond b is a double bond, R₁ is α-OH, R₂ is CH₂OH, and R₃ is CO₂H, then R₄ is not CH₃;
when bond a is a single bond, bond b is a double bond, R₁ is α-OH, R₂ and R₃ are CO₂H or CO₂Me, then R₄ is not CH₃;
when bond a is a single bond, bond b is a double bond, R₁ is α-OCOCH₃, R₂ and R₃ are CO₂H, then R₄ is not CH₃;
when bond a is a single bond, bond b is a double bond, R₁ is β-OH, R₂ and R₃ are CO₂Me, then R₄ is not CH₃;
when bond a is a double bond, bond b is a double bond, R₁ is ═O, R₂ and R₃ are CO₂Me, then R₄ is not CH₃;
when bond a is a single bond, bond b is a double bond, R₁ is α-OH, R₂ and R₃ are CH₂OH, then R₄ is not CH₃;
when bond a is a single bond, bond b is a double bond, R₁ is β-OH, R₂ and R₃ are CH₂OH, then R₄ is not CH₃;
when bond a is a single bond, bond b is a double bond, R₁ is β-OH, R₃ is CO₂H, and R₄ is CH₃, then R₂ is not CHO;
when bond a is a single bond, bond b is a double bond, R₁ is β-OH, R₂ is CH₂OH, and R₄ is CH₃, then R₃ is not CHO, CO₂H, CH₂CH═CH₂, CH₂C≡CH, CH₂C≡CCH₃; CH₂CH₂OH, (CH₂)₄OH, or (CH₂)₆OH;
when bond a is a double bond, bond b is a double bond, R₁ is ═O, R₃ is CO₂H, and R₄ is CH₃, then R₂ is not CH₂OH, or CO₂H;
when bond a is a single bond, bond b is a double bond, R₁ is β-OCOCH₃, R₂ is CH₂OCOCH₃, and R₄ is CH₃, then R₃ is not CO₂H, CH₂CH₂OH, (CH₂)₄OH, or (CH₂)₆OH;
when bond a is a single bond, bond b is a double bond, R₁ is β-OCOCH₂CH₃, R₂ is CH₂OCOCH₂CH₃, and R₄ is CH₃, then R₃ is not CO₂H;
when bond a is a single bond, bond b is a double bond, R₁ is α-OSO₂Ph, R₂ and R₃ are CH₂OSO₂Ph, then R₄ is not CH₃;
when bond a is a single bond, bond b is a double bond, R₁ is β-OCO-4′-Cl-Ph, R₃ is CO₂H, and R₄ is CH₃, then R₂ is not CH₂OCO-4′-Cl-Ph; and
when bond a is a single bond, bond b is a double bond, R₁ is β-OCO-benzyl, R₃ is CO₂H, and R₄ is CH₃, then R₂ is not CH₂OCO-benzyl.

2. A compound according to claim 1, wherein
when bond a is a double bond, R₁ is ═O, ═N—OH, or ═N—OCOCH₃; or when bond a is a single bond, R₁ is OH, NH₂, NHCOCH₃, OCOCH₃, or tetrahydro-2H-pyran-2-yloxy-;
R₂ is CH₂OH, CH₂OCOCH₃, or (tetrahydro-2H-pyran-2-yloxy)methyl-;
R₃ is CO₂H, CH₂OH, CH₂OCOCH₃, CH₂NH₂, CH₂NHCH₂CH₂OH, CN, tetrahydro-2H-pyran-2-yloxy)methyl-, CH═NOH, or CHO; and
R₄ is CH₃, CH₂OH, CH₂NHCH₂CH₂OH, CH₂OOSO₂CH₃, CHO, or CO₂H.

3. A compound according to claim 2, wherein
R₁ is OH, ═O, ═N—OH, ═N—OCOCH₃, NH₂, NHCOCH₃, or OCOCH₃;
R₂ is CH₂OH or CH₂OCOCH₃;
R₃ is CO₂H, CH₂OH, CH₂OCOCH₃, CH₂NH2, CH₂NHCH₂CH₂OH, CN, or CH═NOH; and
R₄ is CH₃, CH₂OH, or CHO.

4. A compound according to claim 2, wherein
R₁ is ═N—OCOCH₃; R₂ is CH₂OCOCH₃; R₃ is CO₂H; and R₄ is CH_{3 [}14];
R₁ is ═N—OCOCH₃; R₂ is CH₂OCOCH₃; R₃ is (tetrahydro-2H-pyran-2-yloxy)methyl-; and R₄ is CH_{3 [}15a];
R₁ is ═N—OCOCH₃; R₂ is CH₂OCOCH₃; R₃ is CH₂OH; and R₄ is CH_{3 [}15b];
R₁ is ═N—OCOCH₃; R₂ is CH₂OCOCH₃; R₃ is CH₂OCOCH₃; and R₄ is CH_{3 [}15c];
R₁ is ═N—OH; R₂ is CH₂OH; R₃ is CO₂H; and R₄ is CH_{3 [}16];
R₁ is ═N—OH; R₂ is CH₂OH; R₃ is (tetrahydro-2H-pyran-2-yloxy)methyl-; and R₄ is CH_{3 [}17a];
R₁ is ═N—OH; R₂ is CH₂OH; R₃ is CH₂OH; and R₄ is CH_{3 [}17b];
R₁ is ═N—OH; R₂ is CH₂OH; R₃ is CH₂OCOCH₃; and R₄ is CH_{3 [}17c];
R₁ is ═O; R₂ is CH₂OH; R₃ is CH₂OH; and R₄ is CH_{3 [}18b];
R₁ is ═O; R₂ is CH₂OH; R₃ is CH₂OCOCH₃; and R₄ is CH_{3 [}18c];
R₁ is OH; R₂ is CH₂OH; R₃ is CH₂OCOCH₃; and R₄ is CH_{3 [}19];
R₁ is tetrahydro-2H-pyran-2-yloxy-; R₂ is (tetrahydro-2H-pyran-2-yloxy)methyl-;
R₃ is CH₂OCOCH₃; and R₄ is CH_{3 [}20];
R₁ is tetrahydro-2H-pyran-2-yloxy-; R₂ is (tetrahydro-2H-pyran-2-yloxy)methyl-;
R₃ is CH₂OH; and R₄ is CH_{3 [}21];
R₁ is NH₂; R₂ is CH₂OH; R₃ is CO₂H; and R₄ is CH_{3 [}22];
R₁ is NH₂; R₂ is CH₂OH; R₃ is CH₂OH; and R₄ is CH_{3 [}23];
R₁ is NHCOCH₃; R₂ is CH₂OCOCH₃; R₃ is CO₂H; and R₄ is CH_{3 [}24];
R₁ is NHCOCH₃; R₂ is CH₂OCOCH₃; R₃ is CH₂OCOCH₃; and R₄ is CH_{3 [}25];
R₁ is NHCOCH₃; R₂ is CH₂OH; R₃ is CO₂H; and R₄ is CH_{3 [}26];
R₁ is NHCOCH₃; R₂ is CH₂OH; R₃ is CH₂OH; and R₄ is CH_{3 [}27];
R₁ is ═N—OCOCH₃; R₂ is CH₂OCOCH₃; R₃ is CHO; and R₄ is CH_{3 [}28];
R₁ is ═N—OCOCH₃; R₂ is CH₂OCOCH₃; R₃ is CH═NOH; and R₄ is CH_{3 [}29];
R₁ is ═N—OH; R₂ is CH₂OH; R₃ is CH═NOH; and R₄ is CH_{3 [}30];
R₁ is NH₂; R₂ is CH₂OH; R₃ is CH₂NH₂; and R₄ is CH_{3 [}31];
R₁ is ═N—OH; R₂ is CH₂OH; R₃ is CHO; and R₄ is CH_{3 [}32];
R₁ is ═N—OH; R₂ is CH₂OH; R₃ is CH₂NHCH₂CH₂OH; and R₄ is CH_{3 [}33];
R₁ is NH₂; R₂ is CH₂OH; R₃ is CH₂NHCH₂CH₂OH; and R₄ is CH_{3 [}34];
R₁ is ═N—OCOCH₃; R₂ is CH₂OCOCH₃; R₃ is CN; and R₄ is CH_{3 [}35];
R₁ is ═N—OH; R₂ is CH₂OH; R₃ is CN; and R₄ is CH_{3 [}36];
R₁ is NH₂; R₂ is CH₂OH; R₃ is CN; and R₄ is CH_{3 [}37];
R₁ is ═O; R₂ is CH₂OCOCH₃; R₃ is CH₂OCOCH₃; and R₄ is CH_{3 [}38];
R₁ is ═N—OCOCH₃, R₂ is CH₂OCOCH₃, R₃ is CO₂H, and R₄ is CH₂OH [39a];
R₁ is ═N—OCOCH₃, R₂ is CH₂OCOCH₃, R₃ is CH₂OH and R₄ is CH₂OH [39b];
R₁ is ═N—OCOCH₃, R₂ is CH₂OCOCH₃, R₃ is CH₂OCOCH₃, and R₄ is CH₂OH [39c];
R₁ is ═N—OCOCH₃, R₂ is CH₂OCOCH₃, R₃ is CN, and R₄ is CH₂OH [39d];
R₁ is ═O, R₂ is CH₂OCOCH₃, R₃ is CH₂OCOCH₃, and R₄ is CH₂OH [40c];
R₁ is ═N—OH, R₂ is CH₂OCOCH₃, R₃ is CO₂H, and R₄ is CH₂OH [41a];
R₁ is ═N—OH, R₂ is CH₂OCOCH₃, R₃ is CH₂OH and R₄ is CH₂OH [41b];
R₁ is ═N—OH, R₂ is CH₂OCOCH₃, R₃ is CH₂OCOCH₃, and R₄ is CH₂OH [41c];
R₁ is ═N—OH, R₂ is CH₂OCOCH₃, R₃ is CN, and R₄ is CH₂OH [41d];
R₁ is ═O, R₂ is CH₂OCOCH₃, R₃ is CO₂H, and R₄ is CH₂OH [42a];
R₁ is ═O, R₂ is CH₂OCOCH₃, R₃ is CH₂OH and R₄ is CH₂OH [42b];
R₁ is ═O, R₂ is CH₂OCOCH₃, R₃ is CH₂OCOCH₃, and R₄ is CH₂OH [42c];
R₁ is ═O, R₂ is CH₂OCOCH₃, R₃ is CN, and R₄ is CH₂OH [42d];
R₁ is OH, R₂ is CH₂OCOCH₃, R₃ is CO₂H, and R₄ is CH₂OH [43a];
R₁ is OH, R₂ is CH₂OCOCH₃, R₃ is CH₂OH and R₄ is CH₂OH [43b];
R₁ is OH, R₂ is CH₂OCOCH₃, R₃ is CH₂OCOCH₃, and R₄ is CH₂OH [43c];
R₁ is OH, R₂ is CH₂OCOCH₃, R₃ is CN, and R₄ is CH₂OH [43d];
R₁ is OH, R₂ is CH₂OCOCH₃, R₃ is CH₂OCOCH₃, and R₄ is CH₂OH [43e];
R₁ is OCOCH₃, R₂ is CH₂OH, R₃ is CH₂OCOCH₃, and R₄ is CH₂OH [43f];
R₁ is NH₂, R₂ is CH₂OCOCH₃, R₃ is CO₂H, and R₄ is CH₂OH [44a];
R₁ is NH₂, R₂ is CH₂OCOCH₃, R₃ is CH₂OH and R₄ is CH₂OH [44b];
R₁ is NH₂, R₂ is CH₂OCOCH₃, R₃ is CH₂OCOCH₃, and R₄ is CH₂OH [44c];
R₁ is NH₂, R₂ is CH₂OCOCH₃, R₃ is CN, and R₄ is CH₂OH [44d];
R₁ is ═N—OCOCH₃, R₂ is CH₂OCOCH₃, R₃ is CH₂OCOCH₃, and R₄ is CH₂OOSO₂CH_{3 [}45c];
R₁ is ═N—OCOCH₃, R₂ is CH₂OCOCH₃, R₃ is CN, and R₄ is CH₂OOSO₂CH₃ [45d];
R₁ is ═N—OH, R₂ is CH₂OH, R₃ is CH₂OH, and R₄ is CH₂OOSO₂CH_{3 [}46b];
R₁ is ═N—OH, R₂ is CH₂OH, R₃ is CN, and R₄ is CH₂OOSO₂CH_{3 [}46d];
R₁ is ═N—OH, R₂ is CH₂OH, R₃ is CH₂OH, and R₄ is CH₂NHCH₂CH₂OH [47b];
R₁ is ═N—OH, R₂ is CH₂OH, R₃ is CN, and R₄ is CH₂NHCH₂CH₂OH [47d];
R₁ is NH₂ R₂ is CH₂OH, R₃ is CH₂OH, and R₄ is CH₂NHCH₂CH₂OH [48b];
R₁ is NH₂, R₂ is CH₂OH, R₃ is CN, and R₄ is CH₂NHCH₂CH₂OH [48d];
R₁ is ═O, R₂ is CH₂OCOCH₃, R₃ is CH₂OCOCH₃, and R₄ is CHO [49a];
R₁ is ═O, R₂ is CH₂OCOCH₃, R₃ is CH₂OCOCH₃, and R₄ is CO₂H [49b];
R₁ is ═O, R₂ is CH₂OH, R₃ is CH₂OH, and R₄ is CHO [50a]; or
R₁ is ═O, R₂ is CH₂OH, R₃ is CH₂OH, and R₄ is CO₂H [50b].

5. A compound of the formula 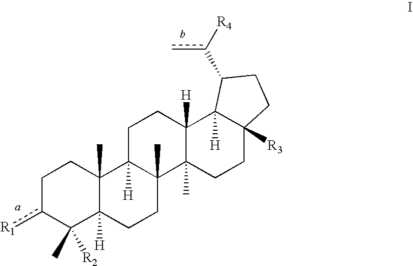
or a pharmaceutically acceptable salt thereof, wherein bond a is a double bond; bond b is a double bond; R₁ is ═O; R₂ is H; R₃ is CH₂OH; and R₄ is CHO [51].

6. A composition comprising a compound according to any one of claim 1, 2 or 5 and a pharmaceutically acceptable carrier, excipient, or diluent.

7. A method of treating a cell proliferative condition in a patient, said method comprising administering to a patient an effective amount of a compound of formula I 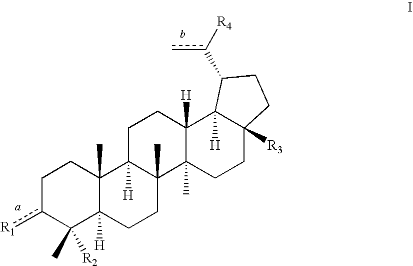
or a pharmaceutically acceptable salt thereof, wherein
each [symbol] is independently a single or double bond;
when bond a is a double bond, R₁ is ═O, ═S, ═N—OH, ═N—OR₅, or ═N—OCOR₅; or when bond a is a single bond, R₁ is H, halogen, NH₂, OH, SH, NHOH, NHR₅, NH(CH₂)ₙX, NR₅R₆, OR₅, OCOR₅, OCO(CH₂)ₙX, OC(O)OR₅, OCO(HC═CH)ₙR₅, OC(O)NHR₅, OC(O)NR₅R₆, OSO₂H, OSO₃H, OSO₂(R₅), OSO₂(CH₂)ₙX, OSi(R₅)ₘ(R₆)₃₋ₘ, SR₅, SCOR₅, SCO(CH₂)ₙX, SC(O)NH₂, SC(O)NHR₅, SC(O)NR₅R₆, NHCOR₅, NHC(O)OR₅, N(R₆)C(O)OR₅, NHC(O)NH₂, NHC(O)NHR₅, NHC(O)NR₅R₆, N(R₆)C(O)NH₂, N(R₆)C(O)NHR₅, N(R₆)C(O)NR₅R₆;
R₂ and R₃ are independently selected from CH₂NH₂, CH₂OH, CH₂SH, CH₂SePh, CH₂X, CH₂NHOH, CN, CHO, CO₂H, CH₂NHOR₅, CH═N—OH, CH═N—OR₅, CH═N—OCOR₅, CH₂NHR₅, CH₂NH(CH₂)ₙX, CH₂NR₅R₆, CH₂OR₅, CH₂OCOR₅, CH₂OC(O)OR₅, CH₂OCO(CH₂)ₙX, CH₂OCO(HC═CH)ₙR₅, CH₂OC(O)NH₂, CH₂OC(O)NHR₅, CH₂OC(O)NR₅R₆, CH₂OSO₂(R₅), CH₂OSO₂(CH₂)ₙX, CH₂OSi(R₅)ₘ(R₆)₃₋ₘ, CH₂SR₅, CH₂SCOR₅, CH₂SCO(CH₂)ₙX, CH₂SC(O)NH₂, CH₂SC(O)NHR₅, CH₂SC(O)NR₅R₆, CH₂NHCOR₅, CH₂NHC(O)OR₅, CH₂NHC(O)NH₂, CH₂NHC(O)NHR₅, CH₂NHC(O)NR₅R₆, CH₂N(R₆)COR₅, CH₂N(R₆)C(O)OR₅, CH₂N(R₆)C(O)NH₂, CH₂N(R₆)C(O)NHR₅, CH₂N(R₆)C(O)NR₅R₆, CH(OR₅)₂, CH(OH)OR₅, CO₂R₅, C(O)NH₂, C(O)NHR₅, C(O)NR₅R₆, C(O)NHOH, or C(O)NHOR₅;
R₄ is selected from CH₃, CH₂NH₂, CH₂OH, CH₂SH, CH₂SePh, CH₂X, CH₂NHOH, CN, CHO, CO₂H, CH₂NHOR₅, CH═N—OH, CH═N—OR₅, CH═N—OCOR₅, CH₂NHR₅, CH₂NH(CH₂)ₙX, CH₂NR₅R₆, CH₂OR₅, CH₂OCOR₅, CH₂OC(O)OR₅, CH₂OCO(CH₂)ₙX, CH₂OCO(HC═CH)R₅, CH₂OC(O)NH₂, CH₂OC(O)NHR₅, CH₂OC(O)NR₅R₆, CH₂OSO₂(R₅), CH₂OSO₂(CH₂)ₙX, CH₂OSi(R₅)ₘ(R₆)₃₋ₘ, CH₂SR₅, CH₂SCOR₅, CH₂SCO(CH₂)ₙX, CH₂SC(O)NH₂, CH₂SC(O)NHR₅, CH₂SC(O)NR₅R₆, CH₂NHCOR₅, CH₂NHC(O)OR₅, CH₂NHC(O)NH₂, CH₂NHC(O)NHR₅, CH₂NHC(O)NR₅R₆, CH₂N(R₆)COR₅, CH₂N(R₆)C(O)OR₅, CH₂N(R₆)C(O)NH₂, CH₂N(R₆)C(O)NHR₅, CH₂N(R₆)C(O)NR₅R₆, CH(OR₅)₂, CH(OH)OR₅, CO₂R₅, C(O)NH₂, C(O)NHR₅, C(O)NR₅R₆, C(O)NHOH, or C(O)NHOR₅;
R₅ and R₆ are independently a straight or branched C₁₋₈ alkyl, aryl-C₁₋₈ alkyl, heterocycle-C₁₋₈ alkyl, cyclo(C₃₋₉)alkyl, aryl, or heterocycle, wherein
the aryl comprises phenyl or a polycyclic aryl group such as naphthyl;
the heterocyle comprises
a five or six membered aromatic heterocycle such as pyridyl, diazinyl, pyrimidinyl, 5-methoxy pyrimidinyl, (1,2,4)triazine-3,5-dione-6-yl, 6-mercaptopyrimidine-4-yl, pyrrolyl, pyrazolyl, imidazolyl, oxazolyl, isoxazolyl, thiazolyl, isothiazolyl, furanyl, or thienyl;
a 3 to 9 membered non-aromatic heterocycle comprising piperazinyl, 4-methyl piperazinyl, pyrrolidinyl, imidazolidinyl, imidazolinyl, thiazolidinyl, thiazolinyl, isothiazolidinyl, isothiazolinyl, pyranyl, or morpholinyl; or
a polycyclic heterocycle such as indolyl, benzthienyl, benzofuranyl, isoindolyl, isobenzothienyl, or isobenzofuranyl;
wherein each alkyl, cycloalkyl, aryl, and heterocycle are each optionally substituted with one or more groups (e.g., 1, 2, or 3 group) which are each independently C₁₋₈ alkyl, C₁₋₈ alkoxy, C₁₋₈ alkylamino, di(C₁₋₈ alkyl)amino, C₁₋₈ alkylamino-C₁₋₈ alkyl, di(C₁₋₆ alkyl)amino-C₁₋₈ alkyl, OC(O)OR₇, OC(O)N(R₇)₂, C(O)OR₇, C(O)N(R₇)₂, S(O)₂R₇, S(O)₂N(R₇)₂, N(R₇)C(O)N(R₇)₂, N(R₇)C(O)OR₇, CN, N₃, NHOH, ═NOH, NH₂, NO₂, OH, SH, F, Cl, Br, or I, wherein each R₇ is independently H or C₁₋₈ alkyl; or
R₅ and R₆ taken together with the carbon atoms to which they are attached form a cyclo(C₃₋₉)alkyl, aryl, or heterocycle, wherein
the aryl comprises a phenyl or a polycyclic aryl group;
the heterocyle comprises
a five or six membered aromatic heterocycle, such as pyridyl, diazinyl, pyrimidinyl, 5-methoxy pyrimidinyl, (1,2,4)triazine-3,5-dione-6-yl, 6-mercaptopyrimidine-4-yl, pyrrolyl, pyrazolyl, imidazolyl, oxazolyl, isoxazolyl, thiazolyl, isothiazolyl, furanyl, or thienyl;
a 3 to 9 membered non-aromatic heterocycle comprising piperazinyl, pyrrolidinyl, imidazolidinyl, imidazolinyl, thiazolidinyl, thiazolinyl, isothiazolidinyl, isothiazolinyl, 4-methyl piperazinyl, pyranyl, or morpholinyl; or
a polycyclic heterocycle such as indolyl, benzthienyl, benzofuranyl, isoindolyl, isobenzothienyl, or isobenzofuranyl;
wherein the cycloalkyl, aryl, and heterocycle are each optionally substituted with one or more groups (e.g., 1, 2, or 3 groups) which are each independently C₁₋₈ alkyl, C₁₋₈ alkoxy, C₁₋₈ alkylamino, di(C₁₋₈ alkyl)amino, C₁₋₈ alkylamino-C₁₋₈ alkyl, di(C₁₋₆ alkyl)amino-C₁₋₈ alkyl, OC(O)OR₇, OC(O)N(R₇)₂, C(O)OR₇, C(O)N(R₇)₂, S(O)₂R₇, S(O)₂N(R₇)₂, N(R₇)C(O)N(R₇)₂, N(R₇)C(O)OR₇, CN, N₃, NHOH, ═NOH, NH₂, NO₂, OH, SH, F, Cl, Br, or I wherein each R₇ is independently H or C₁₋₈ alkyl;
each X is independently Y or aryl, heterocycle, or cyclo(C₃₋₉)alkyl, wherein the aryl, heterocycle, or cyclo(C₃₋₉)alkyl are each optionally substituted with one or more Y groups (e.g., 1, 2, or 3 Y groups), wherein each Y is independently F, Cl, Br, I, CN, N₃, NHOH, ═NOH, NH₂, OH, SH, NHR₅, NR₅R₆, OR₅, SR₅, CO₂H, CO₂R₅, SO(OH)₂, or SO(OR₅)₂;
each m is independently 0, 1, 2, or 3;
each n is independently 1, 2, 3, 4, or 5;

8. The method according to claim 7, wherein the cell proliferative condition is cancer.

9. The method according to claim 8, wherein the cancer is melanoma, glioblastoma, ovarian carcinoma, colon carcinoma, breast carcinoma, or cervical cancer.

10. A method for inhibiting viral, bacterial or malarial infection in a patient, said method comprising administering to the patient an effective amount of a compound of formula I: 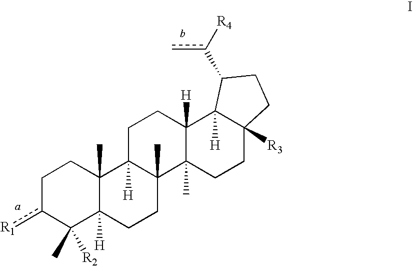
or a pharmaceutically acceptable salt thereof, wherein
each [symbol] is independently a single or double bond;
when bond a is a double bond, R₁ is ═O, ═S, ═N—OH, ═N—OR₅, or ═N—OCOR₅; or when bond a is a single bond, R₁ is H, halogen, NH₂, OH, SH, NHOH, NHR₅, NH(CH₂)ₙX, NR₅R₆, OR₅, OCOR₅, OCO(CH₂)ₙX, OC(O)OR₅, OCO(HC═CH)ₙR₅, OC(O)NHR₅, OC(O)NR₅R₆, OSO₂H, OSO₃H, OSO₂(R₅), OSO₂(CH₂)ₙX, OSi(R₅)ₘ(R₆)₃₋ₘ, SR₅, SCOR₅, SCO(CH₂)ₙX, SC(O)NH₂, SC(O)NHR₅, SC(O)NR₅R₆, NHCOR₅, NHC(O)OR₅, N(R₆)C(O)OR₅, NHC(O)NH₂, NHC(O)NHR₅, NHC(O)NR₅R₆, N(R₆)C(O)NH₂, N(R₆)C(O)NHR₅, N(R₆)C(O)NR₅R₆;
R₂ and R₃ are independently selected from CH₂NH₂, CH₂OH, CH₂SH, CH₂SePh, CH₂X, CH₂NHOH, CN, CHO, CO₂H, CH₂NHOR₅, CH═N—OH, CH═N—OR₅, CH═N—OCOR₅, CH₂NHR₅, CH₂NH(CH₂)ₙX, CH₂NR₅R₆, CH₂OR₅, CH₂OCOR₅, CH₂OC(O)OR₅, CH₂OCO(CH₂)ₙX, CH₂OCO(HC═CH)ₙR₅, CH₂OC(O)NH₂, CH₂OC(O)NHR₅, CH₂OC(O)NR₅R₆, CH₂OSO₂(R₅), CH₂OSO₂(CH₂)ₙX, CH₂OSi(R₅)ₘ(R₆)₃₋ₘ, CH₂SR₅, CH₂SCOR₅, CH₂SCO(CH₂)ₙX, CH₂SC(O)NH₂, CH₂SC(O)NHR₅, CH₂SC(O)NR₅R₆, CH₂NHCOR₅, CH₂NHC(O)OR₅, CH₂NHC(O)NH₂, CH₂NHC(O)NHR₅, CH₂NHC(O)NR₅R₆, CH₂N(R₆)COR₅, CH₂N(R₆)C(O)OR₅, CH₂N(R₆)C(O)NH₂, CH₂N(R₆)C(O)NHR₅, CH₂N(R₆)C(O)NR₅R₆, CH(OR₅)₂, CH(OH)OR₅, CO₂R₅, C(O)NH₂, C(O)NHR₅, C(O)NR₅R₆, C(O)NHOH, or C(O)NHOR₅;
R₄ is selected from CH₃, CH₂NH₂, CH₂OH, CH₂SH, CH₂SePh, CH₂X, CH₂NHOH, CN, CHO, CO₂H, CH₂NHOR₅, CH═N—OH, CH═N—OR₅, CH═N—OCOR₅, CH₂NHR₅, CH₂NH(CH₂)ₙX, CH₂NR₅R₆, CH₂OR₅, CH₂OCOR₅, CH₂OC(O)OR₅, CH₂OCO(CH₂)ₙX, CH₂OCO(HC═CH)ₙR₅, CH₂OC(O)NH₂, CH₂OC(O)NHR₅, CH₂OC(O)NR₅R₆, CH₂OSO₂(R₅), CH₂OSO₂(CH₂)ₙX, CH₂OSi(R₅)ₘ(R₆)₃₋ₘ, CH₂SR₅, CH₂SCOR₅, CH₂SCO(CH₂)ₙX, CH₂SC(O)NH₂, CH₂SC(O)NHR₅, CH₂SC(O)NR₅R₆, CH₂NHCOR₅, CH₂NHC(O)OR₅, CH₂NHC(O)NH₂, CH₂NHC(O)NHR₅, CH₂NHC(O)NR₅R₆, CH₂N(R₆)COR₅, CH₂N(R₆)C(O)OR₅, CH₂N(R₆)C(O)NH₂, CH₂N(R₆)C(O)NHR₅, CH₂N(R₆)C(O)NR₅R₆, CH(OR₅)₂, CH(OH)OR₅, CO₂R₅, C(O)NH₂, C(O)NHR₅, C(O)NR₅R₆, C(O)NHOH, or C(O)NHOR₅;
R₅ and R₆ are independently a straight or branched C₁₋₈ alkyl, aryl-C₁₋₈ alkyl, heterocycle-C₁₋₈ alkyl, cyclo(C₃₋₉)alkyl, aryl, or heterocycle, wherein
the aryl comprises phenyl or a polycyclic aryl group such as naphthyl;
the heterocyle comprises
a five or six membered aromatic heterocycle such as pyridyl, diazinyl, pyrimidinyl, 5-methoxy pyrimidinyl, (1,2,4)triazine-3,5-dione-6-yl, 6-mercaptopyrimidine-4-yl, pyrrolyl, pyrazolyl, imidazolyl, oxazolyl, isoxazolyl, thiazolyl, isothiazolyl, furanyl, or thienyl;
a 3 to 9 membered non-aromatic heterocycle comprising piperazinyl, 4-methyl piperazinyl, pyrrolidinyl, imidazolidinyl, imidazolinyl, thiazolidinyl, thiazolinyl, isothiazolidinyl, isothiazolinyl, pyranyl, or morpholinyl; or
a polycyclic heterocycle such as indolyl, benzthienyl, benzofuranyl, isoindolyl, isobenzothienyl, or isobenzofuranyl;
wherein each alkyl, cycloalkyl, aryl, and heterocycle are each optionally substituted with one or more groups (e.g., 1, 2, or 3 group) which are each independently C₁₋₈ alkyl, C₁₋₈ alkoxy, C₁₋₈ alkylamino, di(C₁₋₈ alkyl)amino, C₁₋₈ alkylamino-C₁₋₈ alkyl, di(C₁₋₆ alkyl)amino-C₁₋₈ alkyl, OC(O)OR₇, OC(O)N(R₇)₂, C(O)OR₇, C(O)N(R₇)₂, S(O)₂R₇, S(O)₂N(R₇)₂, N(R₇)C(O)N(R₇)₂, N(R₇)C(O)OR₇, CN, N₃, NHOH, ═NOH, NH₂, NO₂, OH, SH, F, Cl, Br, or I, wherein each R₇ is independently H or C₁₋₈ alkyl; or
R₅ and R₆ taken together with the carbon atoms to which they are attached form a cyclo(C₃₋₉)alkyl, aryl, or heterocycle, wherein
the aryl comprises a phenyl or a polycyclic aryl group;
the heterocyle comprises
a five or six membered aromatic heterocycle, such as pyridyl, diazinyl, pyrimidinyl, 5-methoxy pyrimidinyl, (1,2,4)triazine-3,5-dione-6-yl, 6-mercaptopyrimidine-4-yl, pyrrolyl, pyrazolyl, imidazolyl, oxazolyl, isoxazolyl, thiazolyl, isothiazolyl, furanyl, or thienyl;
a 3 to 9 membered non-aromatic heterocycle comprising piperazinyl, pyrrolidinyl, imidazolidinyl, imidazolinyl, thiazolidinyl, thiazolinyl, isothiazolidinyl, isothiazolinyl, 4-methyl piperazinyl, pyranyl, or morpholinyl; or
a polycyclic heterocycle such as indolyl, benzthienyl, benzofuranyl, isoindolyl, isobenzothienyl, or isobenzofuranyl;
wherein the cycloalkyl, aryl, and heterocycle are each optionally substituted with one or more groups (e.g., 1, 2, or 3 groups) which are each independently C₁₋₈ alkyl, C₁₋₈ alkoxy, C₁₋₈ alkylamino, di(C₁₋₈ alkyl)amino, C₁₋₈ alkylamino-C₁₋₈ alkyl, di(C₁₋₆ alkyl)amino-C₁₋₈ alkyl, OC(O)OR₇, OC(O)N(R₇)₂, C(O)OR₇, C(O)N(R₇)₂, S(O)₂R₇, S(O)₂N(R₇)₂, N(R₇)C(O)N(R₇)₂, N(R₇)C(O)OR₇, CN, N₃, NHOH, ═NOH, NH₂, NO₂, OH, SH, F, Cl, Br, or I wherein each R₇ is independently H or C₁₋₈ alkyl;
each X is independently Y or aryl, heterocycle, or cyclo(C₃₋₉)alkyl, wherein the aryl, heterocycle, or cyclo(C₃₋₉)alkyl are each optionally substituted with one or more Y groups (e.g., 1, 2, or 3 Y groups), wherein each Y is independently F, Cl, Br, I, CN, N₃, NHOH, ═NOH, NH₂, OH, SH, NHR₅, NR₅R₆, OR₅, SR₅, CO₂H, CO₂R₅, SO(OH)₂, or SO(OR₅)₂;
each m is independently 0, 1, 2, or 3;
each n is independently 1, 2, 3, 4, or 5;

11. A method for treating inflammation in a patient, said method comprising administering to a patient an effective amount of a compound of formula I 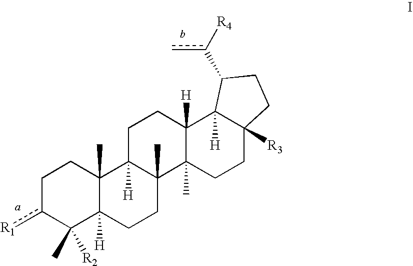
or a pharmaceutically acceptable salt thereof, wherein
each [symbol] is independently a single or double bond;
when bond a is a double bond, R₁ is ═O, ═S, ═N—OH, ═N—OR₅, or ═N—OCOR₅; or when bond a is a single bond, R₁ is H, halogen, NH₂, OH, SH, NHOH, NHR₅, NH(CH₂)ₙX, NR₅R₆, OR₅, OCOR₅, OCO(CH₂)ₙX, OC(O)OR₅, OCO(HC═CH)ₙR₅, OC(O)NHR₅, OC(O)NR₅R₆, OSO₂H, OSO₃H, OSO₂(R₅), OSO₂(CH₂)ₙX, OSi(R₅)ₘ(R₆)₃₋ₘ, SR₅, SCOR₅, SCO(CH₂)ₙX, SC(O)NH₂, SC(O)NHR₅, SC(O)NR₅R₆, NHCOR₅, NHC(O)OR₅, N(R₆)C(O)OR₅, NHC(O)NH₂, NHC(O)NHR₅, NHC(O)NR₅R₆, N(R₆)C(O)NH₂, N(R₆)C(O)NHR₅, N(R₆)C(O)NR₅R₆;
R₂ and R₃ are independently selected from CH₂NH₂, CH₂OH, CH₂SH, CH₂SePh, CH₂X, CH₂NHOH, CN, CHO, CO₂H, CH₂NHOR₅, CH═N—OH, CH═N—OR₅, CH═N—OCOR₅, CH₂NHR₅, CH₂NH(CH₂)ₙX, CH₂NR₅R₆, CH₂OR₅, CH₂OCOR₅, CH₂OC(O)OR₅, CH₂OCO(CH₂)ₙX, CH₂OCO(HC═CH)ₙR₅, CH₂OC(O)NH₂, CH₂OC(O)NHR₅, CH₂OC(O)NR₅R₆, CH₂OSO₂(R₅), CH₂OSO₂(CH₂)ₙX, CH₂OSi(R₅)ₘ(R₆)₃₋ₘ, CH₂SR₅, CH₂SCOR₅, CH₂SCO(CH₂)ₙX, CH₂SC(O)NH₂, CH₂SC(O)NHR₅, CH₂SC(O)NR₅R₆, CH₂NHCOR₅, CH₂NHC(O)OR₅, CH₂NHC(O)NH₂, CH₂NHC(O)NHR₅, CH₂NHC(O)NR₅R₆, CH₂N(R₆)COR₅, CH₂N(R₆)C(O)OR₅, CH₂N(R₆)C(O)NH₂, CH₂N(R₆)C(O)NHR₅, CH₂N(R₆)C(O)NR₅R₆, CH(OR₅)₂, CH(OH)OR₅, CO₂R₅, C(O)NH₂, C(O)NHR₅, C(O)NR₅R₆, C(O)NHOH, or C(O)NHOR₅;
R₄ is selected from CH₃, CH₂NH₂, CH₂OH, CH₂SH, CH₂SePh, CH₂X, CH₂NHOH, CN, CHO, CO₂H, CH₂NHOR₅, CH═N—OH, CH═N—OR₅, CH═N—OCOR₅, CH₂NHR₅, CH₂NH(CH₂)ₙX, CH₂NR₅R₆, CH₂OR₅, CH₂OCOR₅, CH₂OC(O)OR₅, CH₂OCO(CH₂)ₙX, CH₂OCO(HC═CH)ₙR₅, CH₂OC(O)NH₂, CH₂OC(O)NHR₅, CH₂OC(O)NR₅R₆, CH₂OSO₂(R₅), CH₂OSO₂(CH₂)ₙX, CH₂OSi(R₅)ₘ(R₆)₃₋ₘ, CH₂SR₅, CH₂SCOR₅, CH₂SCO(CH₂)ₙX, CH₂SC(O)NH₂, CH₂SC(O)NHR₅, CH₂SC(O)NR₅R₆, CH₂NHCOR₅, CH₂NHC(O)OR₅, CH₂NHC(O)NH₂, CH₂NHC(O)NHR₅, CH₂NHC(O)NR₅R₆, CH₂N(R₆)COR₅, CH₂N(R₆)C(O)OR₅, CH₂N(R₆)C(O)NH₂, CH₂N(R₆)C(O)NHR₅, CH₂N(R₆)C(O)NR₅R₆, CH(OR₅)₂, CH(OH)OR₅, CO₂R₅, C(O)NH₂, C(O)NHR₅, C(O)NR₅R₆, C(O)NHOH, or C(O)NHOR₅;
R₅ and R₆ are independently a straight or branched C₁₋₈ alkyl, aryl-C₁₋₈ alkyl, heterocycle-C₁₋₈ alkyl, cyclo(C₃₋₉)alkyl, aryl, or heterocycle, wherein
the aryl comprises phenyl or a polycyclic aryl group such as naphthyl;
the heterocyle comprises
a five or six membered aromatic heterocycle such as pyridyl, diazinyl, pyrimidinyl, 5-methoxy pyrimidinyl, (1,2,4)triazine-3,5-dione-6-yl, 6-mercaptopyrimidine-4-yl, pyrrolyl, pyrazolyl, imidazolyl, oxazolyl, isoxazolyl, thiazolyl, isothiazolyl, furanyl, or thienyl;
a 3 to 9 membered non-aromatic heterocycle comprising piperazinyl, 4-methyl piperazinyl, pyrrolidinyl, imidazolidinyl, imidazolinyl, thiazolidinyl, thiazolinyl, isothiazolidinyl, isothiazolinyl, pyranyl, or morpholinyl; or
a polycyclic heterocycle such as indolyl, benzthienyl, benzofuranyl, isoindolyl, isobenzothienyl, or isobenzofuranyl;
wherein each alkyl, cycloalkyl, aryl, and heterocycle are each optionally substituted with one or more groups (e.g., 1, 2, or 3 group) which are each independently C₁₋₈ alkyl, C₁₋₈ alkoxy, C₁₋₈ alkylamino, di(C₁₋₈ alkyl)amino, C₁₋₈ alkylamino-C₁₋₈ alkyl, di(C₁₋₆ alkyl)amino-C₁₋₈ alkyl, OC(O)OR₇, OC(O)N(R₇)₂, C(O)OR₇, C(O)N(R₇)₂, S(O)₂R₇, S(O)₂N(R₇)₂, N(R₇)C(O)N(R₇)₂, N(R₇)C(O)OR₇, CN, N₃, NHOH, ═NOH, NH₂, NO₂, OH, SH, F, Cl, Br, or I, wherein each R₇ is independently H or C₁₋₈ alkyl; or
R₅ and R₆ taken together with the carbon atoms to which they are attached form a cyclo(C₃₋₉)alkyl, aryl, or heterocycle, wherein
the aryl comprises a phenyl or a polycyclic aryl group;
the heterocyle comprises
a five or six membered aromatic heterocycle, such as pyridyl, diazinyl, pyrimidinyl, 5-methoxy pyrimidinyl, (1,2,4)triazine-3,5-dione-6-yl, 6-mercaptopyrimidine-4-yl, pyrrolyl, pyrazolyl, imidazolyl, oxazolyl, isoxazolyl, thiazolyl, isothiazolyl, furanyl, or thienyl;
a 3 to 9 membered non-aromatic heterocycle comprising piperazinyl, pyrrolidinyl, imidazolidinyl, imidazolinyl, thiazolidinyl, thiazolinyl, isothiazolidinyl, isothiazolinyl, 4-methyl piperazinyl, pyranyl, or morpholinyl; or
a polycyclic heterocycle such as indolyl, benzthienyl, benzofuranyl, isoindolyl, isobenzothienyl, or isobenzofuranyl;
wherein the cycloalkyl, aryl, and heterocycle are each optionally substituted with one or more groups (e.g., 1, 2, or 3 groups) which are each independently C₁₋₈ alkyl, C₁₋₈ alkoxy, C₁₋₈ alkylamino, di(C₁₋₈ alkyl)amino, C₁₋₈ alkylamino-C₁₋₈ alkyl, di(C₁₋₆ alkyl)amino-C₁₋₈ alkyl, OC(O)OR₇, OC(O)N(R₇)₂, C(O)OR₇, C(O)N(R₇)₂, S(O)₂R₇, S(O)₂N(R₇)₂, N(R₇)C(O)N(R₇)₂, N(R₇)C(O)OR₇, CN, N₃, NHOH, ═NOH, NH₂, NO₂, OH, SH, F, Cl, Br, or I wherein each R₇ is independently H or C₁₋₈ alkyl;
each X is independently Y or aryl, heterocycle, or cyclo(C₃₋₉)alkyl, wherein the aryl, heterocycle, or cyclo(C₃₋₉)alkyl are each optionally substituted with one or more Y groups (e.g., 1, 2, or 3 Y groups), wherein each Y is independently F, Cl, Br, I, CN, N₃, NHOH, ═NOH, NH₂, OH, SH, NHR₅, NR₅R₆, OR₅, SR₅, CO₂H, CO₂R₅, SO(OH)₂, or SO(OR₅)₂;
each m is independently 0, 1, 2, or 3;
each n is independently 1, 2, 3, 4, or 5;

12. The method according to any one of claim 7, 10, or 11 wherein the compound selected from the group consisting of:
R₁ is ═N—OCOCH₃; R₂ is CH₂OCOCH₃; R₃ is CO₂H; and R₄ is CH_{3 [}14];
R₁ is ═N—OCOCH₃; R₂ is CH₂OCOCH₃; R₃ is (tetrahydro-2H-pyran-2-yloxy)methyl-; and R₄ is CH_{3 [}15a];
R₁ is ═N—OCOCH₃; R₂ is CH₂OCOCH₃; R₃ is CH₂OH; and R₄ is CH_{3 [}15b];
R₁ is ═N—OCOCH₃; R₂ is CH₂OCOCH₃; R₃ is CH₂OCOCH₃; and R₄ is CH_{3 [}15c];
R₁ is ═N—OH; R₂ is CH₂OH; R₃ is CO₂H; and R₄ is CH_{3 [}16];
R₁ is ═N—OH; R₂ is CH₂OH; R₃ is (tetrahydro-2H-pyran-2-yloxy)methyl-; and R₄ is CH_{3 [}17a];
R₁ is ═N—OH; R₂ is CH₂OH; R₃ is CH₂OH; and R₄ is CH_{3 [}17b];
R₁ is ═N—OH; R₂ is CH₂OH; R₃ is CH₂OCOCH₃; and R₄ is CH_{3 [}17c];
R₁ is ═O; R₂ is CH₂OH; R₃ is CH₂OH; and R₄ is CH_{3 [}18b];
R₁ is ═O; R₂ is CH₂OH; R₃ is CH₂OCOCH₃; and R₄ is CH_{3 [}18c];
R₁ is OH; R₂ is CH₂OH; R₃ is CH₂OCOCH₃; and R₄ is CH_{3 [}19];
R₁ is tetrahydro-2H-pyran-2-yloxy-; R₂ is (tetrahydro-2H-pyran-2-yloxy)methyl-; R₃ is CH₂OCOCH₃; and R₄ is CH_{3 [}20];
R₁ is tetrahydro-2H-pyran-2-yloxy-; R₂ is (tetrahydro-2H-pyran-2-yloxy)methyl-;
R₃ is CH₂OH; and R₄ is CH_{3 [}21];
R₁ is NH₂; R₂ is CH₂OH; R₃ is CO₂H; and R₄ is CH_{3 [}22];
R₁ is NH₂; R₂ is CH₂OH; R₃ is CH₂OH; and R₄ is CH_{3 [}23];
R₁ is NHCOCH₃; R₂ is CH₂OCOCH₃; R₃ is CO₂H; and R₄ is CH_{3 [}24];
R₁ is NHCOCH₃; R₂ is CH₂OCOCH₃; R₃ is CH₂OCOCH₃; and R₄ is CH_{3 [}25];
R₁ is NHCOCH₃; R₂ is CH₂OH; R₃ is CO₂H; and R₄ is CH_{3 [}26];
R₁ is NHCOCH₃; R₂ is CH₂OH; R₃ is CH₂OH; and R₄ is CH_{3 [}27];
R₁ is ═N—OCOCH₃; R₂ is CH₂OCOCH₃; R₃ is CHO; and R₄ is CH_{3 [}28];
R₁ is ═N—OCOCH₃; R₂ is CH₂OCOCH₃; R₃ is CH═NOH; and R₄ is CH_{3 [}29];
R₁ is ═N—OH; R₂ is CH₂OH; R₃ is CH═NOH; and R₄ is CH_{3 [}30];
R₁ is NH₂; R₂ is CH₂OH; R₃ is CH₂NH₂; and R₄ is CH_{3 [}31];
R₁ is ═N—OH; R₂ is CH₂OH; R₃ is CHO; and R₄ is CH_{3 [}32];
R₁ is ═N—OH; R₂ is CH₂OH; R₃ is CH₂NHCH₂CH₂OH; and R₄ is CH_{3 [}33];
R₁ is NH₂; R₂ is CH₂OH; R₃ is CH₂NHCH₂CH₂OH; and R₄ is CH_{3 [}34];
R₁ is ═N—OCOCH₃; R₂ is CH₂OCOCH₃; R₃ is CN; and R₄ is CH_{3 [}35];
R₁ is ═N—OH; R₂ is CH₂OH; R₃ is CN; and R₄ is CH_{3 [}36];
R₁ is NH₂; R₂ is CH₂OH; R₃ is CN; and R₄ is CH_{3 [}37];
R₁ is ═O; R₂ is CH₂OCOCH₃; R₃ is CH₂OCOCH₃; and R₄ is CH_{3 [}38];
R₁ is ═N—OCOCH₃, R₂ is CH₂OCOCH₃, R₃ is CO₂H, and R₄ is CH₂OH [39a];
R₁ is ═N—OCOCH₃, R₂ is CH₂OCOCH₃, R₃ is CH₂OH and R₄ is CH₂OH [39b];
R₁ is ═N—OCOCH₃, R₂ is CH₂OCOCH₃, R₃ is CH₂OCOCH₃, and R₄ is CH₂OH [39c];
R₁ is ═N—OCOCH₃, R₂ is CH₂OCOCH₃, R₃ is CN, and R₄ is CH₂OH [39d];
R₁ is ═O, R₂ is CH₂OCOCH₃, R₃ is CH₂OCOCH₃, and R₄ is CH₂OH [40c];
R₁ is ═N—OH, R₂ is CH₂OCOCH₃, R₃ is CO₂H, and R₄ is CH₂OH [41a];
R₁ is ═N—OH, R₂ is CH₂OCOCH₃, R₃ is CH₂OH and R₄ is CH₂OH [41b];
R₁ is ═N—OH, R₂ is CH₂OCOCH₃, R₃ is CH₂OCOCH₃, and R₄ is CH₂OH [41c];
R₁ is ═N—OH, R₂ is CH₂OCOCH₃, R₃ is CN, and R₄ is CH₂OH [41d];
R₁ is ═O, R₂ is CH₂OCOCH₃, R₃ is CO₂H, and R₄ is CH₂OH [42a];
R₁ is ═O, R₂ is CH₂OCOCH₃, R₃ is CH₂OH and R₄ is CH₂OH [42b];
R₁ is ═O, R₂ is CH₂OCOCH₃, R₃ is CH₂OCOCH₃, and R₄ is CH₂OH [42c];
R₁ is ═O, R₂ is CH₂OCOCH₃, R₃ is CN, and R₄ is CH₂OH [42d];
R₁ is OH, R₂ is CH₂OCOCH₃, R₃ is CO₂H, and R₄ is CH₂OH [43a];
R₁ is OH, R₂ is CH₂OCOCH₃, R₃ is CH₂OH and R₄ is CH₂OH [43b];
R₁ is OH, R₂ is CH₂OCOCH₃, R₃ is CH₂OCOCH₃, and R₄ is CH₂OH [43c];
R₁ is OH, R₂ is CH₂OCOCH₃, R₃ is CN, and R₄ is CH₂OH [43d];
R₁ is OH, R₂ is CH₂OCOCH₃, R₃ is CH₂OCOCH₃, and R₄ is CH₂OH [43e];
R₁ is OCOCH₃, R₂ is CH₂OH, R₃ is CH₂OCOCH₃, and R₄ is CH₂OH [43f];
R₁ is NH₂, R₂ is CH₂OCOCH₃, R₃ is CO₂H, and R₄ is CH₂OH [44a];
R₁ is NH₂, R₂ is CH₂OCOCH₃, R₃ is CH₂OH and R₄ is CH₂OH [44b];
R₁ is NH₂, R₂ is CH₂OCOCH₃, R₃ is CH₂OCOCH₃, and R₄ is CH₂OH [44c];
R₁ is NH₂, R₂ is CH₂OCOCH₃, R₃ is CN, and R₄ is CH₂OH [44d];
R₁ is ═N—OCOCH₃, R₂ is CH₂OCOCH₃, R₃ is CH₂OCOCH₃, and R₄ is CH₂OOSO₂CH_{3 [}45c];
R₁ is ═N—OCOCH₃, R₂ is CH₂OCOCH₃, R₃ is CN, and R₄ is CH₂OOSO₂CH₃ [45d];
R₁ is ═N—OH, R₂ is CH₂OH, R₃ is CH₂OH, and R₄ is CH₂OOSO₂CH_{3 [}46b];
R₁ is ═N—OH, R₂ is CH₂OH, R₃ is CN, and R₄ is CH₂OOSO₂CH_{3 [}46d];
R₁ is ═N—OH, R₂ is CH₂OH, R₃ is CH₂OH, and R₄ is CH₂NHCH₂CH₂OH [47b];
R₁ is ═N—OH, R₂ is CH₂OH, R₃ is CN, and R₄ is CH₂NHCH₂CH₂OH [47d];
R₁ is NH₂ R₂ is CH₂OH, R₃ is CH₂OH, and R₄ is CH₂NHCH₂CH₂OH [48b];
R₁ is NH₂, R₂ is CH₂OH, R₃ is CN, and R₄ is CH₂NHCH₂CH₂OH [48d];
R₁ is ═O, R₂ is CH₂OCOCH₃, R₃ is CH₂OCOCH₃, and R₄ is CHO [49a];
R₁ is ═O, R₂ is CH₂OCOCH₃, R₃ is CH₂OCOCH₃, and R₄ is CO₂H [49b];
R₁ is ═O, R₂ is CH₂OH, R₃ is CH₂OH, and R₄ is CHO [50a]; or
R₁ is ═O, R₂ is CH₂OH, R₃ is CH₂OH, and R₄ is CO₂H [50b].

13. The method according to any one of claim 7, 10, or 11 wherein the compound is selected from the group consisting of:
bond a is a single bond, bond b is a double bond, R₁ is α-OH, R₂ is CH₂OH, R₃ is CO₂H, and R₄ is CH₃;
when bond a is a single bond, bond b is a double bond, R₁ is α-OH, R₂ and R₃ are CO₂H or CO₂Me, then R₄ is not CH₃;
when bond a is a single bond, bond b is a double bond, R₁ is α-OCOCH₃, R₂ and R₃ are CO₂H, then R₄ is not CH₃;
when bond a is a single bond, bond b is a double bond, R₁ is β-OH, R₂ and R₃ are CO₂Me, then R₄ is not CH₃;
when bond a is a double bond, bond b is a double bond, R₁ is ═O, R₂ and R₃ are CO₂Me, then R₄ is not CH₃;
when bond a is a single bond, bond b is a double bond, R₁ is α-OH, R₂ and R₃ are CH₂OH, then R₄ is not CH₃;
when bond a is a single bond, bond b is a double bond, R₁ is β-OH, R₂ and R₃ are CH₂OH, then R₄ is not CH₃;
when bond a is a single bond, bond b is a double bond, R₁ is β-OH, R₃ is CO₂H, and R₄ is CH₃, then R₂ is not CHO;
when bond a is a single bond, bond b is a double bond, R₁ is β-OH, R₂ is CH₂OH, and R₄ is CH₃, then R₃ is not CHO, CO₂H, CH₂CH═CH₂, CH₂C≡CH, CH₂C≡CCH₃, CH₂CH₂OH, (CH₂)₄OH, or (CH₂)₆OH;
when bond a is a double bond, bond b is a double bond, R₁ is ═O, R₃ is CO₂H, and R₄ is CH₃, then R₂ is not CH₂OH, or CO₂H;
when bond a is a single bond, bond b is a double bond, R₁ is β-OCOCH₃, R₂ is CH₂OCOCH₃, and R₄ is CH₃, then R₃ is not CO₂H, CH₂CH₂OH, (CH₂)₄OH, or (CH₂)₆OH;
when bond a is a single bond, bond b is a double bond, R₁ is β-OCOCH₂CH₃, R₂ is CH₂OCOCH₂CH₃, and R₄ is CH₃, then R₃ is not CO₂H;
when bond a is a single bond, bond b is a double bond, R₁ is α-OSO₂Ph, R₂ and R₃ are CH₂OSO₂Ph, then R₄ is not CH₃;
when bond a is a single bond, bond b is a double bond, R₁ is β-OCO-4′-Cl-Ph, R₃ is CO₂H, and R₄ is CH₃, then R₂ is not CH₂OCO-4′-Cl-Ph; and
when bond a is a single bond, bond b is a double bond, R₁ is β-OCO-benzyl, R₃ is CO₂H, and R₄ is CH₃, then R₂ is not CH₂OCO-benzyl.

14. A method of treating a cell proliferative condition in a patient, said method comprising administering to a patient an effective amount of a compound of formula I 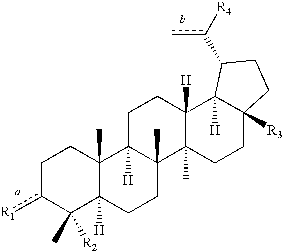
or a pharmaceutically acceptable salt thereof, wherein bond a is a double bond; bond b is a double bond; R₁ is ═O; R₂ is H; R₃ is CH₂OH; and R₄ is CHO [51].

15. A method for inhibiting viral, bacterial or malarial infection in a patient, said method comprising administering to the patient an effective amount of a compound of formula I: 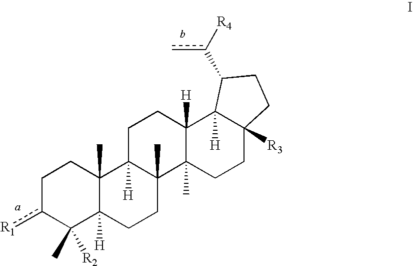
or a pharmaceutically acceptable salt thereof, wherein bond a is a double bond; bond b is a double bond; R₁ is ═O; R₂ is H; R₃ is CH₂OH; and R₄ is CHO [51].

16. A method for treating inflammation in a patient, said method comprising administering to a patient an effective amount of a compound of formula I 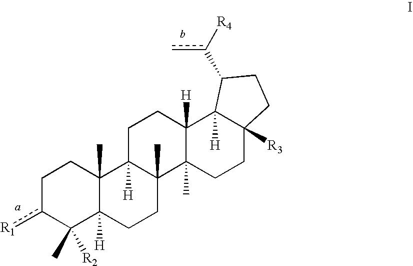
or a pharmaceutically acceptable salt thereof, wherein bond a is a double bond; bond b is a double bond; R₁ is ═O; R₂ is H; R₃ is CH₂OH; and R₄ is CHO [51].
